(19) United States
(12) Patent Application Publication
(10) Pub. No.: US 2017/0306050 A1
(43) Pub. Date: Oct. 26, 2017

(54) COMPOSITIONS AND METHODS FOR TREATING CANCER, INFLAMMATORY DISEASES AND AUTOIMMUNE DISEASES

(71) Applicant: Gilead Sciences, Inc., Foster City, CA (US)

(72) Inventors: Jeremiah D. Degenhardt, San Mateo, CA (US); David Gossage, Seattle, WA (US); Andrew Greenstein, San Francisco, CA (US); Vladi Juric, San Francisco, CA (US); Amanda Mikels-Vigdal, San Carlos, CA (US); Victoria Smith, Burlingame, CA (US); Maria Vaysberg, Los Altos, CA (US); Peng Yue, Foster City, CA (US)

(21) Appl. No.: 15/482,452

(22) Filed: Apr. 7, 2017

Related U.S. Application Data
(60) Provisional application No. 62/408,673, filed on Oct. 14, 2016.
(60) Provisional application No. 62/373,974, filed on Aug. 11, 2016.
(60) Provisional application No. 62/320,441, filed on Apr. 8, 2016.

(51) Int. Cl. C07K 16/40; C07K 16/28; C07K 16/30; A61K 39/00

(52) U.S. Cl. CPC C07K 16/40; C12Y 304/24035; C07K 16/3046; C07K 16/2827; C07K 16/3015; C07K 2317/34; A61K 2039/505; A61K 2039/545; A61K 2039/507; C07K 2317/56; C07K 2317/565; C07K 2317/76

(57) ABSTRACT

The present disclosure provides compositions and methods of use comprising a matrix metalloproteinase-9 (MMP9) binding protein, alone or in combination with one or more additional therapeutic agents for the treatment or prevention of diseases and conditions.

### CROSS-REFERENCE TO RELATED APPLICATIONS

This application claims the benefit of U.S. Provisional Application Ser. No. 62/408,673 filed Oct. 14, 2016, 62/373,974 filed on Aug. 11, 2016, and 62/320,441 filed on Apr. 8, 2016, all of which are hereby incorporated by reference in their entirety for all purposes.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is GILE_120_02US_ST25.TXT. The text file is about 76 KB, was created on Apr. 7, 2017, and is being submitted electronically via EFS-Web.

### FIELD OF THE INVENTION

This present application provides the treatment and prevention of inflammatory diseases.

### BACKGROUND OF THE INVENTION

Immune factors or components may play a role in many diseases or conditions such as cystic fibrosis (CF), cancers, autoimmune diseases and inflammatory diseases. Some studies have suggested that neutrophils, macrophages, and T cells are involved in the infectious and pulmonary pathology of CF, accounting for the majority of CF mortality (Rieber, N. et al. Current concepts of immune dysregulation in cystic fibrosis. The International Journal of Biochemistry & Cell Biology (2014) 52: 108-112).

Cancer cells release chemical signals that lure immune cells such as macrophages and granulocytes to infiltrate the tumor. Once inside the tumor, these immune cells secrete cytokines that promote angiogenesis, which in turn provides the oxygen and nutrients necessary for the tumor to survive and grow. Inflammation might also promote metastasis by producing chemicals that help tumor cells become untethered (Lamagna, C et al. Dual role of macrophages in tumor growth and angiogenesis. Journal of leukocyte biology (2006) 80(4): 705-713).

Autoimmune diseases arise when the immune system becomes dysregulated, mistaking the body's own cells as invaders and attacking these cells. Dysregulation of the innate immune system, on the other hand, could cause inflammation. The immune response is activated even though the body has not been exposed to autoantibodies or antigens. These inflammatory disorders can result in intense episodes of inflammation with such symptoms as fever, rash, and swelling in the joints.

There is a need for safe and effective treatment and prevention of undesired inflammation and immune responses.

### SUMMARY OF THE INVENTION

The present application provides methods of treating or preventing a disease or condition in a subject in need thereof. In one aspect, the application provides a method of treating or preventing a disease or condition in a subject in need thereof, comprising administering to the subject an effective amount of an MMP9 binding protein, and optionally an effective amount of an additional therapeutic agent, thereby treating or preventing the disease or condition in the subject.

The application also provides pharmaceutical compositions comprising a pharmaceutically acceptable excipient, diluent or carrier; an anti-MMP9 antibody or antigen binding fragment thereof and optionally an additional therapeutic agent.

The application also provides kits comprising an anti-MMP9 antibody or antigen binding fragment thereof, and optionally an additional therapeutic agent.

In one embodiment of any of the compositions, kits, or methods for treating or preventing a disease or condition, the MMP9 binding protein is an anti-MMP9 antibody or antigen binding fragment thereof. In certain embodiments, the anti-MMP9 antibody or antigen binding fragment thereof binds to an epitope of MMP9. In certain embodiment, the epitope comprises amino acid residues 104-119, residues 159-166, or residues 191-202 of SEQ ID NO: 27. In another embodiment, the epitope comprises E111, D113, R162, or 1198 of SEQ ID NO: 27. In some embodiments, the anti-MMP9 antibody or antigen binding fragment thereof competes for binding to MMP9 with a protein, wherein the protein binds to amino acid residues 104-119, residues 159-166, or residues 191-202 of SEQ ID NO: 27. In one embodiment, the protein is an antibody having at least about 95%, 96%, 97%, 98%, 99% or greater identity to the amino acid sequences selected from the group consisting of SEQ ID NOs: 7, 12, 13, 14, 15, 16, 17, and 18.

In one embodiment of any of the compositions, kits, or methods for treating or preventing a disease or condition, the anti-MMP9 antibody or antigen binding fragment thereof comprises a heavy chain variable (VH) region comprising a complementarity determining region (CDR) having an amino acid sequence selected from the group consisting of SEQ ID NOs: 13, 14 and 15. In another embodiment, the anti-MMP9 antibody or antigen binding fragment thereof comprises a light chain variable (VL) region having a complementarity determining region (CDR) having an amino acid sequence selected from the group consisting of SEQ ID NOs: 16, 17 and 18. In another embodiment, the anti-MMP9 antibody or antigen binding fragment thereof comprises a VH region comprising an amino acid sequence selected from the group consisting of SEQ ID NOs: 3, 5, 6, 7 and 8. In another embodiment, the anti-MMP9 antibody or antigen binding fragment thereof comprises a VL region having an amino acid sequence selected from the group consisting of SEQ ID NOs: 4, 9, 10, 11 and 12. In one embodiment, the anti-MMP9 antibody or antigen binding fragment thereof comprises a VH region comprising the amino acid sequence set forth in SEQ ID NO: 7 and a VL region comprising the amino acid sequence set forth in SEQ ID NO: 12.

In one embodiment of any of the compositions or methods for treating or preventing a disease or condition, the anti-MMP9 antibody or antigen binding fragment thereof is humanized, chimeric or human. In another embodiment, the anti-MMP9 antibody or antigen binding fragment thereof inhibits the enzymatic activity of MMP9. In some embodiments, the inhibition is non-competitive. In certain embodiments, the anti-MMP9 antibody or antigen binding fragment thereof inhibits MMP9 proteolysis. In another embodiment, the anti-MMP9 antibody or antigen binding fragment thereof inhibits activation of MMP9.

In one embodiment of any of the compositions or methods for treating or preventing a disease or condition, the disease or condition comprises myeloid cell-associated inflammation; cystic fibrosis; non-cystic fibrosis bronchiectasis; sarcoidosis; idiopathic pulmonary fibrosis; tuberculosis; a cancer, e.g., a cancer selected from the group consisting of breast cancer, pancreatic cancer, esophagogastric adenocarcinoma, non-small cell lung cancer, lung squamous cell carcinoma, lung adenocarcinoma, gastric adenocarcinoma, colorectal carcinoma, pancreatic adenocarcinoma, head and neck squamous cell carcinoma, hepatocellular carcinoma, colorectal cancer, colorectal adenocarcinoma and hepatocellular carcinoma; an autoimmune or inflammatory disease or condition, e.g., an autoimmune or inflammatory disease or condition selected from the group consisting of rheumatoid arthritis, an inflammatory bowel disease (IBD) including ulcerative colitis (UC), Crohn's disease (CD), or indeterminate colitis; vasculitis, including large vessel vasculitis (e.g., Takayasu arteritis and Giant cell arteritis), medium vessel vasculitis (e.g., Polyarteritis Nodosa and Kawasaki Disease), immune complex small vessel vasculitis (e.g., Cryoglobulinemic vasculitis, IgA vasculitis (Henoch-Schonlein), and hypocomplementemic urticarial vasculitis (anti-C1q vasculitis)), anti-GBM Disease, ANCA-associated small vessel vasculitis (e.g., microscopic polyangiitis, granulomatosis with polyangiitis (Wegner's), and eosinophilic granulomatosis with polyangiitis (Churg-Strauss)); septicemia; multiple sclerosis; muscular dystrophy; lupus; allergy; asthma; or hidradenitis suppurativa. In some embodiment, the disease or condition is cystic fibrosis. In another embodiment, the disease or condition is rheumatoid arthritis, an inflammatory bowel disease (IBD), septicemia, multiple sclerosis, muscular dystrophy, lupus, allergy or asthma. In certain embodiment, the disease or condition is inflammatory bowel disease (IBD), ulcerative colitis (UC), Crohn's disease (CD), or indeterminate colitis. In another embodiment, the disease or condition is vasculitis.

1. In one embodiment of any of the methods for treating or preventing a disease or condition, the anti-MMP9 antibody or antigen binding fragment thereof is administered concurrently or sequentially with the additional therapeutic agent. In another embodiment, the anti-MMP9 antibody or antigen binding fragment thereof and the additional therapeutic agent are administered in one pharmaceutical composition. In yet another embodiment, the anti-MMP9 antibody or antigen binding fragment thereof and the additional therapeutic agent are administered in two distinct pharmaceutical compositions. In one embodiment, the anti-MMP9 antibody or antigen binding fragment thereof is administered at a dose of about 100 mg, of about 150 mg, of about 200 mg, of about 300 mg, or of about 400 mg. In another embodiment, the anti-MMP9 antibody or antigen binding fragment thereof is administered once every week, once every two weeks, or once every three weeks. In certain embodiments, the anti-MMP9 antibody or antigen binding fragment thereof and/or the additional therapeutic agent is administered intravenously, intradermally, or subcutaneously. Some aspect provides the pharmaceutical composition comprising anti-MMP9 antibody or antigen binding fragment and additional therapeutic agents. The pharmaceutical composition may be administered intravenously, intradermally, or subcutaneously; and may be administered once every week, once every two weeks, or once every three weeks. The pharmaceutical composition would be for use in therapy or for use in a method of treating the disease or condition described herein. In other aspect, the pharmaceutical composition comprises an anti-MMP9 antibody or antigen binding fragment and additional therapeutic agents for the manufacture of a medicament for treatment of the disease or condition described herein.

### BRIEF DESCRIPTION OF THE DRAWINGS

FIG. 1A-FIG. 1C shows the specificity of an antibody (Active AB) raised to a neo-epitope created after cleavage of inactive pro-MMP9 to active MMP9. Rabbits were immunized with the peptide NH₂-FQTFEGDC conjugated to keyhole limpet hemocyanin, and the resulting sera were affinity purified. FIG. 1A, Western blot to assess Total Ab (clone L51/82, Biolegend) and Active Ab specificity for pro-MMP9 versus active MMP9. FIG. 1B, immunohistochemistry to assess Total Ab (Abcam 76003) and Active Ab specificity for pro-MMP9 versus active MMP9. FIG. 1C, peptide enzyme-linked immunosorbent assay (ELISA) to assess Active Ab specificity for a peptide corresponding to the N-terminus of active MMP9 (circle) as compared to off-target peptides corresponding to cleavage at the following residue (squares) or the uncleaved MMP9 pro-domain:catalytic domain junction region (triangles).

FIG. 2A-FIG. 2B shows that MMP9 activity is elevated in diseased colon tissue. FIG. 2A, Endogenous active MMP9 levels in ulcerative colitis and Crohn's disease tissues, measured with MMP9 activity assay (GE, MMP-9 Biotrak Activity Assay) in the absence of APMA or other activator. FIG. 2B, Licor Western blots of pro-MMP9 and active MMP9 in non-diseased tissue and in ulcerative colitis and Crohn's disease tissues.

FIG. 3A-FIG. 3B shows correlations between active MMP9 and disease severity by Geboes histological score (FIG. 3A) and between active MMP9 and total MMP9 in matched tissue lysates from ulcerative colitis (red circles), Crohn's disease (green circles), and non-IBD tissues (blue circles) (FIG. 3B).

FIG. 4A-FIG. 4D shows that active MMP9 and inactive α1-antitrypsin are increased in cystic fibrosis lung tissue. FIG. 4A, levels of total MMP9 in lysates from parenchymal lung tissue from cystic fibrosis (CF) patients (squares) compared to non-CF lung tissues (circles). FIG. 4B, levels of active MMP9 in lung samples from cystic fibrosis (CF) patients (squares) compared to normal lung samples (circles). *, p=0.03. FIG. 4C, ratios of cleaved to intact α1-antitrypsin for CF (squares) and normal samples (circles). ****, p=0.0001. FIG. 4D, visualization of intact and inactive (cleaved) α1-antitrypsin by Licor Western blot.

FIG. 5 shows inactivation of α1-antitrypsin by MMP9 in vitro. The schematic details a protocol described in Example 3 to assess the effect of MMP9, AB0045, and/or control isotype antibody on α1-antitrypsin cleavage. Intact α1-antitrypsin is sufficient to inhibit downstream activation of neutrophil elastase, shown by elastin cleavage.

FIG. 6A-FIG. 6B shows the correlation between active MMP9 and α1-antitrypsin cleavage in lysates from parenchymal lung tissue. FIG. 6A, levels of active MMP9 for CF and non-CF patients (line, left axis) and ratios of cleaved:intact α1-antitrypsin (squares, right axis). FIG. 6B, visualization of al antitrypsin cleavage by Licor Western blot.

FIG. 7A-FIG. 7B shows the effectiveness of MMP9 inhibition in an orthotopic murine model of colorectal cancer. FIG. 7A, change in HCT-116 tumor volume after treatment with antibodies inhibiting both mouse and human MMP9 as compared to Isotype control antibody. FIG. 7B, final tumor weight after study completion.

FIG. 8 shows the efficacy of the combination of an anti-MMP9 agent and an anti-TNF agent in a rheumatoid arthritis mouse model. Mean clinical scores over time are shown for mice in a collagen-induced arthritis (CIA) model of rheumatoid arthritis treated with vehicle (blue circle), Control Ig (square), methotrexate (black circle), AB0046 (triangle), Enbrel® (upside down triangle), or combination AB0046 and Enbrel® (diamond).

FIG. 9A-FIG. 9B shows the efficacy of the combination of an anti-MMP9 agent and an anti-TNF agent in a rheumatoid arthritis mouse model. FIG. 9A, Number of paws per group with clinical score<1.5 (mild disease) over time for mice in a collagen-induced arthritis (CIA) model of rheumatoid arthritis treated with vehicle (blue circle), Control Ig (square), AB0046 (triangle), Enbrel® (upside down triangle), or combination AB0046 and Enbrel® (diamond). *, p<0.05 paired t-test to Vehicle; #, p<0.05 paired t-test to Control, Ig, AB0046 or Enbrel. FIG. 9B, Number of paws per group with clinical scores of 0 (no disease) over time (graph legend as for FIG. 9A). *, p=0.052 paired t-test to Vehicle; #, p<0.05 paired t-test to AB005123. Areas under the curve (Total Area) for each treatment group, with lower numbers indicating clinical efficacy of treatment, are shown for both groups.

FIG. 10 shows T cell diversity analyzed by CDR3 sequences of TCRα and TCRβ chains from mice treated with control, αMMP9, αPD-L1, or combination group, as calculated by MiTCR/MiXCR. Results from the analysis suggest combination therapy could potentially increase TCR clonal diversity.

FIG. 11 shows the relative expression of MMP9 in normal, granulomatosis with polyangiitis (Wegener's, GPA), and giant cell arteritis (GCA) arteries.

FIG. 12A shows relative expression of IL6 in transplanted arteries from mice with induced vasculitis treated with isotype or αMMP9 antibodies.

FIG. 12B shows relative expression of IL1b in transplanted arteries from mice with induced vasculitis treated with isotype or αMMP9 antibodies.

FIG. 12C shows relative expression of TNFα in transplanted arteries from mice with induced vasculitis treated with isotype or αMMP9 antibodies.

FIG. 12D shows relative expression of TCR in transplanted arteries from mice with induced vasculitis treated with isotype or αMMP9.

FIG. 12E shows relative expression of IFNγ in transplanted arteries from mice with induced vasculitis treated with isotype or αMMP9 antibodies.

FIG. 12F shows relative expression of IL17 in transplanted arteries from mice with induced vasculitis treated with isotype or αMMP9 antibodies.

FIG. 13A-FIG. 13B shows relative expression of IFNγ (FIG. 13A) and IL17 (FIG. 13B) in transplanted arteries from mice with induced vasculitis treated with isotype or αMMP9 antibodies for the first 7 days after adoptive peripheral blood mononuclear cell (PBMC) transfer.

FIG. 14 shows MMP9 protein levels by Ashcroft Score in a bleomycin-induced lung fibrosis mouse model. No disease control (circle), IgG control (square). **, p=0.008.

FIG. 15 shows the expression of CK5, a marker of lung bronchiolization, in lung tissue of bleomycin-induced lung fibrosis mouse model. Mice were either not treated with bleomycin (saline-treated control) or were treated with bleomycin and indicated antibodies as described in Example 12. *, p<0.05; **, p<0.01; *** p<0.001.

FIG. 16 shows the results of ELISA assay to measure AB0045 bound MMP9. Sputa from two CF patients were incubated with: 1) 50 mg/ml IgG4 control, 2) 50 mg/ml AB0045+protease inhibitor, 3) 50 mg/ml AB0045 and 4) 50 mg/ml AB0045+10 mg/ml HNE for 24 hours at 37° C. Sputum 1 (left-hand bars), Sputum 2 (right-hand bars).

FIG. 17A-FIG. 17B shows MMP9 activity as measured by a peptide proteolysis assay. FIG. 17A, 0.97 mg/mL AB0045 was incubated with 10 mg/ml HNE (0.5 U/ml) for 23 hrs at 37° C. After digestion, the AB0045 mixture was diluted to the concentration denoted on the x-axis, mixed with MMP9, and MMP9 enzymatic activity was measured. AB0045 incubated with HNE (circle); AB0045, no HNE (square). FIG. 17B, 0.97 mg/ml AB0045 was incubated 1:1 (v:v) with sputa from two distinct CF patients for 23 hrs at 37° C. Peptide proteolysis was measured similar to FIG. 17A. AB0045, incubated with sputum #1 (circle); AB0045, incubated with sputum #2 (square); AB0045, no sputum (triangle); no AB0045 (inverted triangle).

FIG. 18A-FIG. 18B show median tumor volume of tumors over 30 days of treatment (FIG. 18A) and final mean tumor volume (FIG. 18B) in an orthotopic, syngeneic tumor model (NeuT). The mice were treated with control IgG antibody, anti-MMP9 antibody, anti-PDL1 antibody, or the combination of anti-MMP9 and anti-PDL1 antibodies. ** p<0.01. FIG. 18A: control IgG (circle); anti-MMP9 (square); anti-PDL1 (triangle); anti-MMP9/anti-PDL1 (inverted triangle).

FIG. 19A-FIG. 19B shows normalized expression of Granzyme B (FIG. 19A) and CD69 (FIG. 19B), two genes associated with effector T cell signature, in an orthotopic, syngeneic tumor model (NeuT) treated with an anti-MMP9 antibody.

FIG. 20 shows change in TCR clonality in an orthotopic, syngeneic tumor model (NeuT) treated with control IgG antibody, anti-MMP9 antibody, anti-PDL1 antibody, or the combination of anti-MMP9 and anti-PDL1 antibodies.

### DETAILED DESCRIPTION OF THE INVENTION

The present application provides compositions and methods for treating and/or preventing a variety of diseases, conditions and disorders, including but not limited to cystic fibrosis, cancer, autoimmune diseases or conditions, inflammatory diseases or conditions, and diseases and conditions associated with MMP9. In one embodiment, the disease or disorder is associated with deregulated MMP9 expression or activity, e.g., MMP9 overexpression.

Practice of the present disclosure employs, unless otherwise indicated, standard methods and conventional techniques in the fields of cell biology, toxicology, molecular biology, biochemistry, cell culture, immunology, oncology, recombinant DNA and related fields as are within the skill of the art. Such techniques are described in the literature and thereby available to those of skill in the art. See, for example, Alberts, B. et al., “Molecular Biology of the Cell,” 5th edition, Garland Science, New York, N.Y., 2008; Voet, D. et al. “Fundamentals of Biochemistry: Life at the Molecular Level,” 3rd edition, John Wiley & Sons, Hoboken, N.J., 2008; Sambrook, J. et al., “Molecular Cloning: A Laboratory Manual,” 3rd edition, Cold Spring Harbor Laboratory Press, 2001; Ausubel, F. et al., “Current Protocols in Molecular Biology,” John Wiley & Sons, New York, 1987 and periodic updates; Freshney, R. I., “Culture of Animal Cells: A Manual of Basic Technique,” 4th edition, John Wiley & Sons, Somerset, N.J., 2000; and the series “Methods in Enzymology,” Academic Press, San Diego, Calif. See also, for example, “Current Protocols in Immunology,” (R. Coico, series editor), Wiley, last updated August 2010.

### Definitions

As used herein, the singular form “a”, “an”, and “the” includes plural references unless indicated otherwise.

Reference to “about” a value or parameter herein refers to the usual error range for the respective value readily known to the skilled person in this technical field. Reference to “about” a value or parameter herein includes (and describes) aspects that are directed to that value or parameter per se. For example, description referring to “about X” includes description of “X.” In certain embodiments, the term “about” includes the indicated amount±1% to 10%. In other embodiments, the term “about” includes the indicated amount±5%. In certain other embodiments, the term “about” includes the indicated amount±1%. In certain other embodiments, the term “about” includes the indicated amount±10%.

As used herein, the term “agent” refers to any molecule, compound, nucleic acid, nucleic acid based moiety, antibody, antibody-based molecule, protein, protein-based molecule and/or substance for use in the prevention, treatment, management and/or diagnosis of a disease or condition.

It is understood that aspects and embodiments of the compositions and methods etc. described herein include “comprising,” “consisting,” and “consisting essentially of” aspects and embodiments.

As used herein, an “immune modulating agent” is an agent capable of modulating the immune response of a subject. In some embodiments, an “immune modulating agent” enhances or increases an immune response and may be referred to as “immunostimulatory.” In other embodiments, an “immune modulating agent” inhibits or reduces an immune response and may be referred to as “immunosuppressive.” In certain embodiments, “immune modulating agents” include adjuvants (substances that enhance the body's immune response to an antigen), vaccines (e.g., cancer vaccines), and those agents capable of modulating the function of immune checkpoints, including the Cytotoxic T-lymphocyte-associated protein 4 (CTLA-4), Lymphocyte-activation gene 3 (LAG-3), Cluster of Differentiation 276 (B7-H3), V-set domain-containing T-cell activation inhibitor 1 (B7-H4), T-cell immunoglobulin and mucin domain 3 (Tim3), B- and T-lymphocyte attenuator (BTLA), killer immunoglobulin receptor (KIR), adenosine A2a receptor (A2aR), Cluster of Differentiation 200 (CD200) and/or Programmed cell death protein 1 (PD-1) pathways.

As used herein, a “recombinant molecule” refers to an expression vector harboring a DNA insert. In certain embodiments, the “recombinant molecule” is designed to express a therapeutic agent.

As used herein, “treat,” treating” and “treatment” or the like refer to stasis or a postponement of development of one or more symptoms associated with a disease or disorder described herein, or ameliorating existing uncontrolled or unwanted symptoms, preventing additional symptoms, or ameliorating or preventing the underlying metabolic causes of symptoms. Thus, the terms denote that a beneficial result has been conferred on a mammalian subject with a disease or symptom, or with the potential to develop such disease or symptom. A response is achieved when the patient experiences partial or total alleviation, or reduction of signs or symptoms of illness, and specifically includes, without limitation, prolongation of survival. The expected progression-free survival times can be measured in months to years, depending on prognostic factors including the number of relapses, stage of disease, and other factors.

The first treatment given for a disease or condition is referred to as the “front-line therapy,” “first-line therapy,” “front-line treatment” or “first-line treatment.” In general, the first-line therapy is typically the one accepted as the best treatment that is available to healthcare provider at the time of treatment. If it doesn't cure the disease, alleviate the symptoms or the extent of the disease, or it causes undesired or severe adverse effects, other treatment may be added or used instead. “First-line therapy” may also be referred to as induction therapy, primary therapy, and primary treatment. Any of the methods of treatment or prevention described herein may be provided as a “first-line therapy.” “Second-line therapy” refers to treatment that is given when initial treatment (first-line therapy) doesn't work, or stops working. Any of the methods of treatment or prevention described herein may be provided as a “second-line therapy.” “Add-on therapy” refers to any treatment given to bolster or enhance the effectiveness of another therapy, e.g., when the first treatment proved not to be hilly effective. Any of the methods of treatment or prevention described herein may be provided as an “add-on therapy.”

A “prophylactically effective amount” refers to an amount effective at the dosages and for periods of time necessary, to achieve the desired prophylactic result. Typically, but not necessarily, since a prophylactic dose is used in subjects prior to or at the earlier stage of disease, the prophylactically effective amount can be less than the therapeutically effective amount.

As used herein, the term “subject” refers to a mammalian subject. Exemplary subjects include, but are not limited to humans, non-human primates, monkeys, dogs, cats, mice, rats, cows, horses, goats and sheep. In certain embodiments, the subject is a human. In some embodiments, the subject has or is being diagnosed as having CF, an inflammatory disease or condition, or an autoimmune disease or condition, and may be treated with the agent or the antibody of the present application. Other embodiments provide that a human in need of treatment with the antibodies of the present application, wherein the human has or is suspected to have CF, an inflammatory disease or condition, or an autoimmune disease or condition.

As used herein, the term “antibody” refers to an isolated or recombinant polypeptide binding agent that comprises peptide sequences (e.g., variable region sequences) that specifically bind an antigenic epitope. The term is used in its broadest sense and specifically covers monoclonal antibodies (including full-length monoclonal antibodies), polyclonal antibodies, human antibodies, humanized antibodies, chimeric antibodies, nanobodies, diabodies, multispecific antibodies (e.g., bispecific antibodies), and antibody fragments including but not limited to Fv, scFv, Fab, Fab′ F(ab′)₂ and Fab₂, so long as they exhibit the desired biological activity. The term “human antibody” refers to antibodies containing sequences of human origin, except for possible non-human CDR regions, and does not imply that the full structure of an immunoglobulin molecule be present, only that the antibody has minimal immunogenic effect in a human (i.e., does not induce the production of antibodies to itself).

An “antibody fragment” comprises a portion of a full-length antibody, for example, the antigen binding or variable region of a full-length antibody. Such antibody fragments may also be referred to herein as “functional fragments: or “antigen-binding fragments”. Examples of antibody fragments include Fab, Fab′, F(ab′)₂, and Fv fragments; diabodies; linear antibodies (Zapata et al. (1995) Protein Eng. 8(10):1057-1062); single-chain antibody molecules; and multi-specific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)₂ fragment that has two antigen combining sites and is still capable of cross-linking antigen.

“Fv” is a minimum antibody fragment containing a complete antigen-recognition and -binding site. This region consists of a dimer of one heavy- and one light-chain variable domain in tight, non-covalent association. It is in this configuration that the three complementarity-determining regions (CDRs) of each variable domain interact to define an antigen-binding site on the surface of the V_{H}V_{L} dimer. Collectively, the six CDRs confer antigen-binding specificity to the antibody. However, even a single variable domain (or an isolated V_{H} or V_{L} region comprising only three of the six CDRs specific for an antigen) has the ability to recognize and bind antigen, although generally at a lower affinity than does the entire Fv fragment.

The “F_{ab}” fragment also contains, in addition to heavy and light chain variable regions, the constant domain of the light chain and the first constant domain (CH₁) of the heavy chain. Fab fragments were originally observed following papain digestion of an antibody. Fab′ fragments differ from Fab fragments in that F(ab′) fragments contain several additional residues at the carboxy terminus of the heavy chain CHI domain, including one or more cysteines from the antibody hinge region. F(ab′)₂ fragments contain two Fab fragments joined, near the hinge region, by disulfide bonds, and were originally observed following pepsin digestion of an antibody. Fab′-SH is the designation herein for Fab′ fragments in which the cysteine residue(s) of the constant domains bear a free thiol group. Other chemical couplings of antibody fragments are also known.

The “light chains” of antibodies (immunoglobulins) from any vertebrate species can be assigned to one of two clearly distinct types, called kappa and lambda, based on the amino acid sequences of their constant domains. Depending on the amino acid sequence of the constant domain of their heavy chains, immunoglobulins can be assigned to five major classes: IgA, IgD, IgE, IgG, and IgM, and several of these may be further divided into subclasses (isotypes), e.g., IgG1, IgG2, IgG3, IgG4, IgA1, and IgA2.

“Single-chain “Fv” or “sFv” or “scFv” antibody fragments comprise the V_{H} and V_{L} domains of antibody, wherein these domains are present in a single polypeptide chain. In some embodiments, the Fv polypeptide further comprises a polypeptide linker between the VH and VL domains, which enables the sFv to form the desired structure for antigen binding. For a review of sFv, see Pluckthun, in The Pharmacology of Monoclonal Antibodies, vol. 113 (Rosenburg and Moore eds.) Springer-Verlag, New York, pp. 269-315 (1994).

The term “diabodies” refers to small antibody fragments with two antigen-binding sites, which fragments comprise a heavy-chain variable domain (VH) connected to a light-chain variable domain (VL) in the same polypeptide chain (VHVL). By using a linker that is too short to allow pairing between the two domains on the same chain, the domains are forced to pair with the complementary domains of another chain, thereby creating two antigen-binding sites. Diabodies are additionally described, for example, in EP 404,097; WO 93111161 and Hollinger et al. (1993) Proc. Natl. Acad. Sci. USA 90:6444-6448.

An “isolated” antibody is one that has been identified and separated and/or recovered from a component of its natural environment. Components of its natural environment may include enzymes, hormones, and other proteinaceous or nonproteinaceous solutes. In some embodiments, an isolated antibody is purified (1) to greater than 95% by weight of antibody as determined by the Lowry method, for example, more than 99% by weight, (2) to a degree sufficient to obtain at least 15 residues of N-terminal or internal amino acid sequence, e.g., by use of a spinning cup sequenator, or (3) to homogeneity by gel electrophoresis (e.g., SDS-PAGE) under reducing or nonreducing conditions, with detection by Coomassie blue or silver stain. The term “isolated antibody” includes an antibody in situ within recombinant cells, since at least one component of the antibody's natural environment will not be present. In certain embodiments, isolated antibody is prepared by at least one purification step.

As used herein, “immunoreactive” refers to antibodies or fragments thereof that are specific to a sequence of amino acid residues (“binding site” or “epitope”), yet if are cross-reactive to other peptides/proteins, are not toxic at the levels at which they are formulated for administration to human use. “Epitope” refers to that portion of an antigen capable of forming a binding interaction with an antibody or antigen binding fragment thereof. An epitope can be a linear peptide sequence (i.e., “continuous”) or can be composed of noncontiguous amino acid sequences (i.e., “conformational” or “discontinuous”). The term “preferentially binds” means that the binding agent binds to the binding site with greater affinity than it binds unrelated amino acid sequences.

Antibodies of the present disclosure can be described in terms of the CDRs of the heavy and light chains. As used herein, the term “CDR” or “complementarity determining region” is intended to mean the non-contiguous antigen combining sites found within the variable region of both heavy and light chain polypeptides. These particular regions have been described by Kabat et al., J. Biol. Chem. 252:6609-6616 (1977); Kabat et al., U.S. Dept. of Health and Human Services, “Sequences of proteins of immunological interest” (1991); by Chothia et al., J. Mol. Biol. 196:901-917 (1987); and MacCallum et al., J. Mol. Biol. 262:732-745 (1996), where the definitions include overlapping or subsets of amino acid residues when compared against each other. Nevertheless, application of either definition to refer to a CDR of an antibody or grafted antibodies or variants thereof is intended to be within the scope of the term as defined and used herein. The amino acid residues which encompass the CDRs as defined by each of the above cited references are set forth below in Table 1A as a comparison.

TABLE 1A
CDR Definitions

| | Kabat¹ | Chothia² | MacCallum³ |
|---|---|---|---|
| V_{H} CDR1 | 31-35 | 26-32 | 30-35 |
| V_{H} CDR2 | 50-65 | 53-55 | 47-58 |
| V_{H} CDR3 | 95-102 | 96-101 | 93-101 |
| V_{L} CDR1 | 24-34 | 26-32 | 30-36 |
| V_{L} CDR2 | 50-56 | 50-52 | 46-55 |
| V_{L} CDR3 | 89-97 | 91-96 | 89-96 |

¹Residue numbering follows the nomenclature of Kabat et al., supra
²Residue numbering follows the nomenclature of Chothia et al., supra
³Residue numbering follows the nomenclature of MacCallum et al., supra

As used herein, the term “framework” when used in reference to an antibody variable region is intended to mean all amino acid residues outside the CDR regions within the variable region of an antibody. A variable region framework is generally a discontinuous amino acid sequence between about 100-120 amino acids in length but is intended to reference only those amino acids outside of the CDRs. As used herein, the term “framework region” is intended to mean each domain of the framework that is separated by the CDRs.

In some embodiments, an antibody is a humanized antibody or a human antibody. Humanized antibodies include human immunoglobulins (recipient antibody) in which residues from a complementary-determining region (CDR) of the recipient are replaced by residues from a CDR of a non-human species (donor antibody) such as mouse, rat or rabbit having the desired specificity, affinity and capacity. Thus, humanized forms of non-human (e.g., murine) antibodies are chimeric immunoglobulins which contain minimal sequence derived from non-human immunoglobulin. The non-human sequences are located primarily in the variable regions, particularly in the complementarity-determining regions (CDRs). In some embodiments, Fv framework residues of the human immunoglobulin are replaced by corresponding non-human residues. Humanized antibodies can also comprise residues that are found neither in the recipient antibody nor in the imported CDR or framework sequences. In certain embodiments, a humanized antibody comprises substantially all of at least one, and typically two, variable domains, in which all or substantially all of the CDRs correspond to those of a non-human immunoglobulin and all or substantially all of the framework regions are those of a human immunoglobulin consensus sequence. For the purposes of the present disclosure, humanized antibodies can also include immunoglobulin fragments, such as Fv, Fab, Fab′, F(ab′)₂ or other antigen-binding subsequences of antibodies.

The humanized antibody can also comprise at least a portion of an immunoglobulin constant region (Fe), typically that of a human immunoglobulin. See, for example, Jones et al. (1986) Nature 321:522-525; Riechmann et al. (1988) Nature 332:323-329; and Presta (1992) Curr. Op. Struct. Biol. 2:593-596.

Methods for humanizing non-human antibodies are known in the art. Generally, a humanized antibody has one or more amino acid residues introduced into it from a source that is non-human. These non-human amino acid residues are often referred to as “import” or “donor” residues, which are typically obtained from an “import” or “donor” variable domain. For example, humanization can be performed essentially according to the method of Winter and co-workers, by substituting rodent CDRs or CDR sequences for the corresponding sequences of a human antibody. See, for example, Jones et al., supra; Riechmann et al., supra and Verhoeyen et al. (1988) Science 239:1534-1536. Accordingly, such “humanized” antibodies include chimeric antibodies (U.S. Pat. No. 4,816,567), wherein substantially less than an intact human variable domain has been substituted by the corresponding sequence from a non-human species. In certain embodiments, humanized antibodies are human antibodies in which some CDR residues and optionally some framework region residues are substituted by residues from analogous sites in rodent antibodies (e.g., murine monoclonal antibodies).

Human antibodies can also be produced, for example, by using phage display libraries. Hoogenboom et al. (1991) J. Mol. Biol, 227:381; Marks et al. (1991) J. Mol. Biol. 222:581. Other methods for preparing human monoclonal antibodies are described by Cole et al. (1985) “Monoclonal Antibodies and Cancer Therapy,” Alan R. Liss, p. 77 and Boerner et al. (1991) J. Immunol. 147:86-95.

Human antibodies can be made by introducing human immunoglobulin loci into transgenic animals (e.g., mice) in which the endogenous immunoglobulin genes have been partially or completely inactivated. Upon immunological challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al. (1992) Bio/Technology 10:779-783 (1992); Lonberg et al. (1994) Nature 368: 856-859; Morrison (1994) Nature 368:812-813; Fishwald et al. (1996) Nature Biotechnology 14:845-851; Neuberger (1996) Nature Biotechnology 14:826; and Lonberg et al. (1995) Intern. Rev. Immunol. 13:65-93.

Antibodies can be affinity matured using known selection and/or mutagenesis methods as described above. In some embodiments, affinity matured antibodies have an affinity which is five times or more, ten times or more, twenty times or more, or thirty times or more than that of the starting antibody (generally murine, rabbit, chicken, humanized or human) from which the matured antibody is prepared.

An antibody can also be a bispecific antibody. Bispecific antibodies are monoclonal, and may be human or humanized antibodies that have binding specificities for at least two different antigens. In the present case, the two different binding specificities can be directed to two different MMPs, or to two different epitopes on a single MMP (e.g., MMP9).

An antibody as disclosed herein can also be an immunoconjugate. Such immunoconjugates comprise an antibody (e.g., to MMP9) conjugated to a second molecule, such as a reporter An immunoconjugate can also comprise an antibody conjugated to a cytotoxic agent such as a chemotherapeutic agent, a toxin (e.g., an enzymatically active toxin of bacterial, fungal, plant, or animal origin, or fragments thereof), or a radioactive isotope (i.e., a radioconjugate).

An antibody that “specifically binds to” or is “specific for” a particular polypeptide or an epitope refers to the selective binding of the antibody to the target antigen or epitope; these terms, and methods for determining specific binding, are well understood in the art. An antibody exhibits “specific binding” for a particular target antigen or epitope if it binds with greater affinity, avidity, more readily, and/or with greater duration to that target antigen or epitope than it does with other substances. In some embodiments, the antibody that specifically binds to the polypeptide or epitope is one that that binds to that particular polypeptide or epitope without substantially binding to any other polypeptide or polypeptide epitope. In some embodiments, the provided antibodies specifically bind to human MMP9 or other target with a dissociation constant (Kd) equal to or lower than 100 nM, optionally lower than 10 nM, optionally lower than 1 nM, optionally lower than 0.5 nM, optionally lower than 0.1 nM, optionally lower than 0.01 nM, or optionally lower than 0.005 nM, in certain examples, between 0.1 and 0.2 nM, or between 0.1 and 10 pM, e.g., between 0.4 and 9 pm, such as between 0.4 and 8.8 pm, in the form of monoclonal antibody, scFv, Fab, or other form of antibody measured at a temperature of about 4° C., 25° C., 37° C. or 42° C.

The antibodies for use with the presently provided methods, compositions, and combinations can include but are not limited to any of the antibodies described herein, including antibodies and antibody fragments, including those containing any combination of the various exemplified heavy and light chains, heavy and light chain variable regions, and CDRs.

All references cited herein, including patent applications and publications, are hereby incorporated by reference in their entirety.

### MMP9 Binding Proteins

Embodiments of the present application include or use MMP9 binding proteins, e.g., anti-MMP9 antibodies and fragments thereof that inhibit MMP9 processing or activity, including but not limited to any of the MMP9 binding proteins described herein.

MMP9 degrades basement membrane collagen and other extracellular matrix (ECM) components (Kessenbrock K, et al., “Matrix metalloproteinases: regulators of the tumor microenvironment.” Cell 2010; 141 (1):52-67). Matrix degradation contributes to pathology in multiple diseases, including arthritis, cancer, and ulcerative colitis (Roy R, et al., “Matrix metalloproteinases as novel biomarkers and potential therapeutic targets in human cancer.” J Clin Oncol 2009; 27 (31):5287-97). Broad-spectrum matrix metalloproteinase inhibitors such as Marimastat are efficacious in animal models of inflammation and cancer (Watson S A, et al., “Inhibition of tumour growth by marimastat in a human xenograft model of gastric cancer: relationship with levels of circulating CEA.” Br J Cancer 1999; 81 (1):19-23; Sykes A P, et al., “The effect of an inhibitor of matrix metalloproteinases on colonic inflammation in a trinitrobenzenesulphonic acid rat model of inflammatory bowel disease.” Aliment Pharmacol Ther 1999; 13 (11):1535-42). Such pan inhibitors, however, can cause musculoskeletal side effects including joint stiffness, inflammation, and pain in the hands, arms, and shoulders, collectively referred to as musculoskeletal syndrome (MSS), typically at or near efficacious dose levels of Marimastat in humans (Peterson J T. “The importance of estimating the therapeutic index in the development of matrix metalloproteinase inhibitors.” Cardiovasc Res 2006; 69 (3):677-87; Tierney G M, et al. “A pilot study of the safety and effects of the matrix metalloproteinase inhibitor marimastat in gastric cancer.” Eur J Cancer 1999; 35 (4):563-8; and Wojtowicz-Fraga S, et al. “Phase I trial of Marimastat, a novel matrix metalloproteinase inhibitor, administered orally to patients with advanced lung cancer.” J Clin Oncol 1998; 16 (6):2150-6). The symptoms are dose- and time-dependent, and reversible shortly after cessation of treatment with the pan-MMP inhibitor (Wojtowicz-Fraga S, 1998; Nemunaitis J, et al., “Combined analysis of studies of the effects of the matrix metalloproteinase inhibitor marimastat on serum tumor markers in advanced cancer: selection of a biologically active and tolerable dose for longer-term studies.” Clin Cancer Res 1998; 4 (5):1101-9; Hutchinson J W et al., “Dupuytren's disease and frozen shoulder induced by treatment with a matrix metalloproteinase inhibitor.” The Journal of Bone and Joint Surgery, British Volume 1998; 80 (5):907-8. Marimastat and other pan-MMP inhibitors of the same class are zinc chelators; Peterson J T, 2006. The homozygous MMP9 knockout mouse displays no MSS-like symptoms or MSS-like tissue changes; and Vu T H, et al., “MMP-9/gelatinase B is a key regulator of growth plate angiogenesis and apoptosis of hypertrophic chondrocytes” Cell 1998; 93(3):411-22).

Abnormal activity of certain MMPs plays a role in tumor growth, metastasis, inflammation, autoimmunity, and vascular disease (see, for example, Hu et al. (2007) Nature Reviews: Drug Discovery 6:480-498). One notable source of MMP9 is tumor-associated macrophages (TAMs), which support metastasis and invasion in a complex co-activation loop via paracrine interaction with the primary tumor cells. This combination of the proteolytic breakdown of physical barriers to cell invasion plus liberation of factors that activate growth and angiogenesis paves the way for tumor expansion, with the accompanying development of neovascularization to support tumor outgrowth.

MMP9 is a target of oncogenic signaling pathways such as RAS/RAF, PI3K/AKT/NFκB, and WNT/beta-catenin and functions as an upstream regulator of these pathways via modulation of integrin and receptor tyrosine kinase function. MMP9 is elevated in a wide variety of tumor types and MMP9 levels are correlated with poor prognosis in many cancers, including gastric, lung, and colorectal cancer. MMP9 is also implicated in chemoresistance and is upregulated upon loss of several tumor suppressors. MMP9 is upregulated in many diverse tumor types and can promote primary growth and distal invasion of cancerous cells.

It can be desirable to inhibit the activity of one or more MMPs in certain therapeutic settings. However, the activity of certain other MMPs, e.g., MMP2, is often required for normal function and/or is protective against disease. Since most MMP inhibitors are targeted to the conserved catalytic domain and, as a result, inhibit a number of different MMPS, use of available MMP inhibitors has caused side effects due to the inhibition of essential, non-pathogenically-related MMPs. These side effects may likely be also due to general zinc chelation caused by many of these inhibitors, including inhibiting zinc-requiring enzymes more broadly.

Challenges were associated with developing inhibitors specific to a particular MMP or select MMPs due to the fact that inhibition of enzymatic activity via substrate-competitive mechanisms generally requires that the inhibitor be targeted to the catalytic domain. Homologies in MMP catalytic domains can cause inhibitors to react with more than one MMP. MMP9 binding proteins described herein include agents, including therapeutic reagents, such as antibodies and antigen-binding fragments thereof, that specifically inhibit the catalytic activity of a single MMP or a select plurality of MMPs, such as MMP9 and that do not react with or inhibit certain other MMPs or any other MMPs. Also among the provided embodiments are methods and uses of the same for treatment of various diseases, including cystic fibrosis, cancers, autoimmune diseases and conditions, and inflammatory diseases and conditions. In certain embodiments, the MMP9 binding proteins of this disclosure binds the general large catalytic domain, but does not bind in the substrate pocket, and appears to be acting via other, allosteric mechanisms (e.g., certain MMP9 binding proteins of this disclosure do not compete with substrate for binding, and inhibit independently of the presence of substrate or substrate concentration).

Certain embodiments of the compositions, kits and methods of this application utilize binding proteins, e.g., antibodies and fragments (e.g., antigen-binding fragments) thereof, that bind to the matrix metalloproteinase-9 (MMP9) protein (also referred to as gelatinase-B). In one embodiment, they bind to a human MMP9, such as the human MMP9 having an amino acid sequence set forth in SEQ ID NO: 27 or SEQ ID NO: 28. The binding proteins of the present disclosure generally comprise an immunoglobulin (Ig) heavy chain (or functional fragment thereof) and an Ig light chain (or functional fragment thereof).

The disclosure further provides MMP9 binding proteins that bind specifically to MMP9 and not to other matrix metalloproteinases such as MMP1, MMP2, MMP3, MMP7, MMP9, MMP10, MMP12, and MMP13 (see also WO 2012/027721, WO 2013/130078 and WO 2013/130905, each of which is herein incorporated in its entirety). Such specific MMP9 binding proteins are generally not significantly or detectably cross-reactive with non-MMP9 matrix metalloproteinases. MMP9 binding proteins that specifically bind MMP9 find use in applications in which it is necessary or desirable to obtain specific modulation (e.g., inhibition) of MMP9 without directly affecting the activity of other matrix metalloproteinases.

In certain embodiments of the present disclosure, an anti-MMP9 antibody is an inhibitor of the activity of MMP9, and it can be a specific inhibitor of MMP9. In particular, the MMP9 binding proteins disclosed herein are useful for inhibition of MMP9 while allowing normal function of other, related matrix metalloproteinases. “An inhibitor of MMP9” or “inhibitor of MMP9 activity” can be an antibody or an antigen binding fragment thereof that directly or indirectly inhibits activity of MMP9, including but not limited to enzymatic processing, inhibiting action of MMP9 on it substrate (e.g., by inhibiting substrate binding, substrate cleavage, and the like), and the like.

In one embodiment, the anti-MMP9 antibody or antigen binding fragment thereof inhibits the enzymatic activity of MMP9. In some embodiments, the inhibition is non-competitive. In certain embodiments, the anti-MMP9 antibody or antigen binding fragment thereof inhibits MMP9 proteolysis. In another embodiment, the anti-MMP9 antibody or antigen binding fragment thereof inhibits activation of MMP9.

In some embodiments, whereas treatment with pan-MMP inhibitors, such as the small-molecule pan inhibitors Marimastat, results in symptoms of musculoskeletal disease, such as musculoskeletal syndrome (MSS), which can cause substantial effects on gait, posture and willingness to move, and profound histological damage to joints, specific inhibition of MMP9, e.g., with the antibodies or antigen-binding fragments thereof in the present application, does not cause such symptoms and does not induce MSS.

The present disclosure also provides MMP9 binding proteins that specifically bind to non-mouse MMP9, such as human MMP9, Cynomolgus monkey MMP9, and rat MMP9.

The present disclosure also provides MMP9 binding proteins (e.g., anti-MMP9 antibodies and functional fragments thereof) that act as non-competitive inhibitors. A “non-competitive inhibitor” refers to an inhibitor binds at site away from substrate binding site of an enzyme, and thus can bind the enzyme and effect inhibitory activity regardless of whether or not the enzyme is bound to its substrate. Such non-competitive inhibitors can, for example, provide for a level of inhibition that can be substantially independent of substrate concentration. MMP9 binding proteins (e.g., antibodies and functional fragments thereof) of the present disclosure include those that bind MMP9, e.g., human MMP9, and having a heavy chain polypeptide (or functional fragment thereof) that has at least about 80%, 85%, 90%, 95% or more amino acid sequence identity to a heavy chain polypeptide disclosed herein. In some example, MMP9 binding proteins (e.g., antibodies and functional fragments thereof) of the present disclosure include those that bind MMP9, e.g., human MMP9, and having a light chain polypeptide (or functional fragment thereof) that has at least about 90%, 95%, 97%, 98%, 99% or more amino acid sequence identity to a light chain polypeptide disclosed herein.

MMP9 binding proteins (e.g., antibodies and functional fragments thereof) of the present disclosure include those that bind MMP9, e.g., human MMP9, and having a light polypeptide (or functional fragment thereof) that has at least about 80%, 85%, 90%, 95% or more amino acid sequence identity to a heavy chain polypeptide disclosed herein.

MMP9 binding proteins (e.g., antibodies and functional fragments thereof) of the present disclosure include those that bind MMP9, e.g., human MMP9, and have a heavy chain polypeptide (or functional fragment thereof) having the complementarity determining regions (“CDRs”) of heavy chain polypeptide and the CDRs of a light chain polypeptide (or functional fragment thereof) as disclosed herein.

“Homology” or “identity” or “similarity” as used herein in the context of nucleic acids and polypeptides refers to the relationship between two polypeptides or two nucleic acid molecules based on an alignment of the amino acid sequences or nucleic acid sequences, respectively. Homology and identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology/similarity or identity refers to a function of the number of identical or similar amino acids at positions shared by the compared sequences. In comparing two sequences, the absence of residues (amino acids or nucleic acids) or presence of extra residues also decreases the identity and homology/similarity.

As used herein, “identity” means the percentage of identical nucleotide or amino acid residues at corresponding positions in two or more sequences when the sequences are aligned to maximize sequence matching, i.e., taking into account gaps and insertions. Sequences are generally aligned for maximum correspondence over a designated region, e.g., a region at least about 20, 25, 30, 35, 40, 45, 50, 55, 60, 65 or more amino acids or nucleotides in length, and can be up to the full-length of the reference amino acid or nucleotide. For sequence comparison, typically one sequence acts as a reference sequence, to which test sequences are compared. When using a sequence comparison algorithm, test and reference sequences are input into a computer program, subsequence coordinates are designated, if necessary, and sequence algorithm program parameters are designated. The sequence comparison algorithm then calculates the percent sequence identity for the test sequence(s) relative to the reference sequence, based on the designated program parameters.

Examples of algorithms that are suitable for determining percent sequence identity are the BLAST and BLAST 2.0 algorithms, which are described in Altschul et al. (1990) J. Mol. Biol. 215: 403-410 and Altschul et al. (1977) Nucleic Acids Res. 25: 3389-3402, respectively. Software for performing BLAST analyses is publicly available through the National Center for Biotechnology Information (www.ncbi.nlm.nih.gov). Further exemplary algorithms include ClustalW (Higgins D., et al. (1994) Nucleic Acids Res 22: 4673-4680), available at www.ebi.ac.uk/Tools/clustalw/index.html.

Residue positions which are not identical can differ by conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine.

Accordingly, the present disclosure provides, for example, antibodies or antigen binding fragments thereof, comprising a heavy chain variable region polypeptide having at least 80%, 85%, 90%, 95%, 96%, 97%, 98%, 99% or greater amino acid sequence identity to an amino acid sequence of a heavy chain variable region described herein (e.g., SEQ ID NOS: 1 or 5-8), and a variable light chain polypeptide having at least 80%, 85%, 90%, 95%, 96%, 97%, 98%, 99% or greater amino acid sequence identity to an amino acid sequence of a light chain polypeptide as set forth herein (e.g., SEQ ID NOS: 2 or 9-12). In one embodiment, the present disclosure provides antibodies or antigen binding fragments thereof comprising a heavy chain variable region polypeptide having at least about 95%, 96%, 97%, 98%, 99% or greater amino acid sequence identity to an amino acid sequence of a heavy chain variable region as set forth in SEQ ID NO: 7, and a variable light chain polypeptide having at least about 95%, 96%, 97%, 98%, 99% or greater amino acid sequence identity to an amino acid sequence of a light chain polypeptide as set forth in SEQ ID NO: 12. In further examples, the present disclosure provides antibodies or antigen binding fragments thereof comprising a heavy chain variable region polypeptide having at least 80%, 85%, 90%, 95%, 96%, 97%, 98%, 99% or greater amino acid sequence identity to an amino acid sequence of a heavy chain variable region as set forth in SEQ ID NOS: 32, 40, or 47, and a variable light chain polypeptide having at least 80%, 85%, 90%, 95%, 96%, 97%, 98%, 99% or greater amino acid sequence identity to an amino acid sequence of a light chain polypeptide as set forth in SEQ ID NOS: 33, 41, or 48. In some embodiments, the present disclosure provides antibodies or antigen binding fragments thereof comprising a heavy chain variable region polypeptide having at least about 95%, 96%, 97%, 98%, 99% or greater amino acid sequence identity to an amino acid sequence of a heavy chain variable region as set forth in SEQ ID NOS: 32, 40, or 47, and a variable light chain polypeptide having at least about 95%, 96%, 97%, 98%, 99% or greater amino acid sequence identity to an amino acid sequence of a light chain polypeptide as set forth in SEQ ID NOS: 33, 41, or 48. In further embodiments, the present application provides the antibodies or antigen binding fragment thereof that may compete for binding to a protein or antibody comprising an amino acid sequence having at least about 95%, 96%, 97%, 98%, 99% or greater identity to an amino acid sequence as set forth in SEQ ID NO: 7, 12, 13, 14, 15, 16, 17, or 18.

In some embodiments, an anti-MMP9 antibody or binding fragment thereof of the present disclosure binds to one or more processing sites (e.g., sites of proteolytic cleavage) in MMP9, thereby effectively blocking processing of the proenzyme or preproenzyme to the catalytically active enzyme, and thus reducing the proteolytic activity of the MMP9.

In some embodiments, an anti-MMP9 antibody or binding fragment thereof binds to MMP9 with an affinity at least 2 times, at least 5 times, at least 10 times, at least 25 times, at least 50 times, at least 100 times, at least 500 times, or at least 1000 times greater than its binding affinity for another MMP. Binding affinity can be measured by any method known in the art and can be expressed as, for example, on-rate, off-rate, dissociation constant (Kd), equilibrium constant (Keq) or any term in the art.

In some embodiments, an anti-MMP9 antibody according to the present disclosure is one that inhibits the enzymatic (i.e., catalytic) activity of MMP9, such as a non-competitive inhibitor of the catalytic activity of MMP9. In some embodiments, an antibody according to the present disclosure binds within the catalytic domain of MMP9. In additional embodiments, an antibody according to the present disclosure binds outside the catalytic domain of MMP9.

Also provided are antibodies or antigen binding fragments thereof that compete with any one or more of the anti-MMP9 antibodies or antigen binding fragments thereof described herein for binding to MMP9. Thus, the present disclosure contemplates anti-MMP9 antibodies, and functional fragments thereof, that compete for binding with, for example, an antibody having a heavy chain polypeptide of any of SEQ ID NOS: 1 or 5-8, a light chain polypeptide of SEQ ID NOS: 2 or 9-12, or combinations thereof. In one embodiment, the anti-MMP9 antibody, or functional fragment thereof, competes for binding to human MMP9 with the antibody described herein as AB0041. In some embodiments, the anti-MMP9 antibody or functional fragment thereof competes for binding to human MMP9 with the antibody described herein as AB0045. In certain embodiments, the anti-MMP9 antibody or functional fragment thereof competes for binding to human MMP9 with the antibody described herein as AB0046. In additional embodiments, the anti-MMP9 antibody or functional fragment thereof competes for binding to human MMP9 with the antibody described herein as M4. In other embodiments, the anti-MMP9 antibody or functional fragment thereof competes for binding to human MMP9 with the antibody described herein as M12.

Also provided are antibodies and fragments thereof that bind to the same epitope, e.g., MMP9 epitope as any one or more of the antibodies described herein. Also provided are antibodies and fragments that specifically bind to an epitope of MMP9, where the epitope includes an amino acid residue within a specific region of MMP9 or multiple regions of MMP9. Further provided are anti-MMP9 antibody or antigen binding fragment thereof that compete for binding to a protein or antibody that binds to the epitope or region described herein. Such regions can include, for example, structural loops and/or other structural domains of MMP9, such as those shown to be important for binding to exemplary antibodies described herein. Typically, the regions are defined according to amino acid residue positions on the full-length MMP9 sequence, e.g., SEQ ID NO: 27. In some examples, the epitope contains an amino acid residue 104-202 of SEQ ID NO: 27. In one example, the epitope contains an amino acid residue (i.e., one or more amino acid residue(s)) within a region that is residues 104-119 residues 159-166, or residues 191-202 of SEQ ID NO: 27. In some aspects, the epitope includes an amino acid residue (i.e., one or more amino acid residue(s)) within a region of MMP9 that is residues 104-119 of SEQ ID NO: 27, an amino acid residue within a region of MMP9 that is residues 159-166 of SEQ ID NO: 27, and an amino acid residue within a region of MMP9 that is residues 191-202 of SEQ ID NO: 27. In some cases, the epitope includes E111, D113, R162, or 1198 of SEQ ID NO: 27. In some cases, it includes R162 of SEQ ID NO: 27. In some cases, it includes E111, D113, R162, and 1198 of SEQ ID NO: 27.

The amino acid sequence of human MMP9 protein is as follows:

| (SEQ ID NO: 27) | |
|---|---|
| MSLWQPLVLV LLVLGCCFAA PRQRQSTLVL FPGDLRTNLT DRQLAEEYLY | 50 |
| RYGYTRVAEM RGESKSLGPA LLLLQKQLSL PETGELDSAT LKAMRTPRCG | 100 |
| VPDLGRFQTF EGDLKWHHHN ITYWIQNYSE DLPRAVIDDA FARAFALWSA | 150 |
| VTPLTFTRVY SRDADIVIQF GVAEHGDGYP FDGKDGLLAH AFPPGPGIQG | 200 |
| DAHFDDDELW SLGKGVVVPT RFGNADGAAC HFPFIFEGRS YSACTTDGRS | 250 |
| DGLPWCSTTA NYDTDDRFGF CPSERLYTRD GNADGKPCQF PFIFQGQSYS | 300 |
| ACTTDGRSDG YRWCATTANY DRDKLFGFCP TRADSTVMGG NSAGELCVFP | 350 |
| FTFLGKEYST CTSEGRGDGR LWCATTSNFD SDKKWGFCPD QGYSLFLVAA | 400 |
| HEFGHALGLD HSSVPEALMY PMYRFTEGPP LHKDDVNGIR HLYGPRPEPE | 450 |
| PRPPTTTTPQ PTAPPTVCPT GPPTVHPSER PTAGPTGPPS AGPTGPPTAG | 500 |
| PSTATTVPLS PVDDACNVNI FDAIAEIGNQ LYLFKDGKYW RFSEGRGSRP | 550 |
| QGPFLIADKW PALPRKLDSV FEEPLSKKLF FFSGRQVWVY TGASVLGPRR | 600 |
| LDKLGLGADV AQVTGALRSG RGKMLLFSGR RLWRFDVKAQ MVDPRSASEV | 650 |
| DRMFPGVPLD THDVFQYREK AYFCQDRFYW RVSSRSELNQ VDQVGYVTYD | 700 |
| ILQCPED | |

Protein domains of MMP9 are indicated below:

| Amino Acid # | Feature |
|---|---|
| 1-19 | Signal Peptide |
| 38-98 | Peptidoglycan Binding Domain |
| R98/C99 | Cysteine-switch active pocket |
| 112-445 | Zn dependent metalloproteinase domain |
| 223-271 | Fibronectin type II domain (gelatin binding domain) |
| 281-329 | Fibronectin type II domain (gelatin binding domain) |
| 340-388 | Fibronectin type II domain (gelatin binding domain) |
| 400-411 | Zn binding region |
| 521-565 | Hemopexin-like domain |
| 613-659 | Hemopexin-like domain |
| 567-608 | Hemopexin-like domain |
| 661-704 | Hemopexin-like domain |

The amino acid sequence of mature full-length human MMP9 (which is the amino acid sequence of the propolypeptide of SEQ ID NO: 27 without the signal peptide) is:

(SEQ ID NO: 28)
APRQRQSTLVL FPGDLRTNLT DRQLAEEYLY RYGYTRVAEM
RGESKSLGPA LLLLQKQLSL PETGELDSAT LKAMRTPRCG
VPDLGRFQTF EGDLKWHHHN ITYWIQNYSE DLPRAVIDDA
FARAFALWSA VTPLTFTRVY SRDADIVIQF GVAEHGDGYP
FDGKDGLLAH AFPPGPGIQG DAHFDDDELW SLGKGVVVPT
RFGNADGAAC HFPFIFEGRS YSACTTDGRS DGLPWCSTTA
NYDTDDRFGF CPSERLYTRD GNADGKPCQF PFIFQGQSYS
ACTTDGRSDG YRWCATTANY DRDKLFGFCP TRADSTVMGG
NSAGELCVFP FTFLGKEYST CTSEGRGDGR LWCATTSNFD
SDKKWGFCPD QGYSLFLVAA HEFGHALGLD HSSVPEALMY
PMYRFTEGPP LHKDDVNGIR HLYGPRPEPE PRPPTTTTPQ
PTAPPTVCPT GPPTVHPSER PTAGPTGPPS AGPTGPPTAG
PSTATTVPLS PVDDACNVNI FDAIAEIGNQ LYLFKDGKYW
RFSEGRGSRP QGPFLIADKW PALPRKLDSV FEEPLSKKLF
FFSGRQVWVY TGASVLGPRR LDKLGLGADV AQVTGALRSG
RGKMLLFSGR RLWRFDVKAQ MVDPRSASEV DRMFPGVPLD
THDVFQYREK AYFCQDRFYW RVSSRSELNQ VDQVGYVTYD
ILQCPED

The amino acid sequence of the signal peptide is MSLWQPLVLVLLVLGCCFA (SEQ ID NO: 29).

Also provided are MMP9 polypeptides, including mutant MMP9 polypeptides. Such peptides are useful, for example, in generating and selecting antibodies and fragments as provided herein. Exemplary polypeptides include those having an amino acid sequence containing residues 111-198 of SEQ ID NO: 27, and those having an amino acid sequence containing residues 111-198 of SEQ ID NO: 27 with an amino acid substitution at residue 111, 113, 162, or 198 of SEQ ID NO: 27 or with an amino acid substitution at all such residues. Such polypeptides find use, for example, in selecting antibodies that bind to epitopes containing such residues and/or for which such residues of MMP9 are important for binding, such as those described herein.

The present disclosure contemplates MMP9 binding proteins that bind any portion of MMP9, e.g., human MMP9, including MMP9 binding proteins that preferentially bind MMP9 relative to other MMPs.

Anti-MMP9 antibodies, and functional fragments thereof, can be generated accordingly to methods well known in the art. Exemplary anti-MMP9 antibodies are provided below.

In related embodiments, an anti-MMP9 antibody is a heavy chain variant of AB0041. The amino acid sequences of the variable regions of the AB0041 heavy and light chains have been separately modified, by altering framework region sequences in the heavy and light chain variable regions. The effect of these sequence alterations was to deplete the antibody of human T-cell epitopes, thereby reducing or abolishing its immunogenicity in humans (Antitope, Babraham, UK).

Four heavy-chain variants were constructed, in a human IgG4 heavy chain background containing a S241P amino acid change that stabilizes the hinge domain (Angal et al. (1993) Malec. Immunol. 30:105-108), and are denoted VH1, VH2, VH3 and VH4. The amino acid sequences of their framework regions and CDRs are as follows:

VH1
(SEQ ID NO: 5)
QVQLQESGPGLVKPSETLSLTCTVSGFSLLSYGVHWVRQPPGKGLEWLG
VIWTGGTTNYNSALMSRLTISKDDSKSTVYLKMNSLKTEDTAIYYCARY
YYGMDYWGQGTSVTVSS
VH2
(SEQ ID NO: 6)
QVQLQESGPGLVKPSETLSLTCTVSGFSLLSYGVHWVRQPPGKGLEWLG
VIWTGGTTNYNSALMSRLTISKDDSKNTVYLKMNSLKTEDTAIYYCARY
YYGMDYWGQGTLVTVSS
VH3
(SEQ ID NO: 7)
QVQLQESGPGLVKPSETLSLTCTVSGFSLLSYGVHWVRQPPGKGLEWLG
VIWTGGTTNYNSALMSRFTISKDDSKNTVYLKMNSLKTEDTAIYYCARY
YYGMDYWGQGTLVTVSS
VH4
(SEQ ID NO: 8)
QVQLQESGPGLVKPSETLSLTCTVSGFSLLSYGVHWVRQPPGKGLEWLG
VIWTGGTTNYNSALMSRFTISKDDSKNTLYLKMNSLKTEDTAIYYCARY
YYGMDYWGQGTLVTVSS

In related embodiments, an anti-MMP9 antibody is a light chain variant of AB0041. Four light-chain variants have been constructed, in a human kappa chain background, and are denoted Vk1, Vk2, Vk3 and Vk4. The amino acid sequences of their framework regions and CDRs are as follows:

Vk1
(SEQ ID NO: 9)
DIVMTQSPSFLSASVGDRVTITCKASQDVRNTVAWYQQKTGKAPKLLIY
SSSYRNTGVPDRFTGSGSGTDFTLTISSLQAEDVAVYFCQQHYITPYTF
GGGTKVEIK
Vk2
(SEQ ID NO: 10)
DIVMTQSPSSLSASVGDRVTITCKASQDVRNTVAWYQQKPGKAPKLLIY
SSSYRNTGVPDRFTGSGSGTDFTLTISSLQAEDVAVYFCQQHYITPYTF
GGGTKVEIK
Vk3
(SEQ ID NO: 11)
DIQMTQSPSSLSASVGDRVTITCKASQDVRNTVAWYQQKPGKAPKLLIY
SSSYRNTGVPDRFSGSGSGTDFTLTISSLQAEDVAVYFCQQHYITPYTF
GGGTKVEIK
Vk4
(SEQ ID NO: 12)
DIQMTQSPSSLSASVGDRVTITCKASQDVRNTVAWYQQKPGKAPKLLIY
SSSYRNTGVPDRFSGSGSGTDFTLTISSLQAEDVAVYYCQQHYITPYTF
GGGTKVEIK

According to the present disclosure, the humanized heavy and light chains may be combined in all possible pair-wise combinations to generate a number of functional humanized anti-MMP9 antibodies. For example, provided are antibodies with a heavy chain variable (VH) region having the amino acid sequence set forth in any of SEQ ID NOs: 3, 5, 6, 7, and 8; antibodies having a light chain variable (VL) region having the amino acid sequence set forth in any of SEQ ID NOs: 4, 9, 10, 11, and 12; and antibodies with a heavy chain variable (VH) region having the amino acid sequence set forth in any of SEQ ID NOs: 3, 5, 6, 7, and 8 and a light chain variable (VL) region having the amino acid sequence set forth in any of SEQ ID NOs: 4, 9, 10, 11, and 12, as well as antibodies that compete for binding to MMP9 with such antibodies and antibodies having at least at or about 75%, 80%, 85%, 90%, 91%, 92%, 93%, 94%, 95%, 96%, 97%, 98%, 99% or more sequence identity with such antibodies. In one example, the antibody has a VH region with an amino acid sequence having at least at or about 75%, 80%, 85%, 90%, 91%, 92%, 93%, 94%, 95%, 96%, 97%, 98%, 99% or more sequence identity with SEQ ID NO: 7 and a VL region with an amino acid sequence having at least at or about 75%, 80%, 85%, 90%, 91%, 92%, 93%, 94%, 95%, 96%, 97%, 98%, 99% or more sequence identity with SEQ ID NO: 12, or a VH region of SEQ ID NO: 7 and a VL region of SEQ ID NO: 12. In an additional example, the antibody has a VH region with an amino acid sequence having at least at or about 95%, 96%, 97%, 98%, 99% or more sequence identity with SEQ ID NO: 7. In a further example, the antibody has a VL region with an amino acid sequence having at least at or about 95%, 96%, 97%, 98%, 99% or more sequence identity with SEQ ID NO: 12. In some example, the antibody has a VH region of SEQ ID NO: 7 and a VL region of SEQ ID NO: 12.

Additional heavy chain variable region amino acid sequences having 75% or more, 80% or more, 90% or more, 95% or more, or 99% or more homology to the heavy chain variable region sequences disclosed herein are also provided. Furthermore, additional light chain variable region amino acid sequences having 75% or more, 80% or more, 90% or more, 95% or more, or 99% or more homology to the light chain variable region sequences disclosed herein are also provided.

Additional heavy chain variable region amino acid sequences having 75% or more, 80% or more, 90% or more, 95% or more, or 99% or more sequence identity to the heavy chain variable region sequences disclosed herein are also provided. Furthermore, additional light chain variable region amino acid sequences having 75% or more, 80% or more, 90% or more, 95% or more, or 99% or more sequence identity to the light chain variable region sequences disclosed herein are also provided.

In some embodiments, the CDRs of the heavy chain of anti-MMP9 antibodies disclosed herein have the following amino acid sequences:

CDR1:
(SEQ ID NO: 13)
GFSLLSYGVH
CDR2:
(SEQ ID NO: 14)
VIWTGGTTNYNSALMS
CDR3:
(SEQ ID NO: 15)
YYYGMDY

Thus, among the provided anti-MMP9 antibodies are antibodies having a heavy chain CDR1 region with an amino acid sequence as set forth in SEQ ID NO: 13, antibodies having a heavy chain CDR2 region with an amino acid sequence set forth in SEQ ID NO: 14, and antibodies having a heavy chain CDR3 region with an amino acid sequence as set forth in SEQ ID NO: 15, and antibodies that compete for binding with or bind to the same epitope on MMP9 as such antibodies. In some cases, the antibodies contain VH CDRs having the sequences set forth in SEQ ID NO: 15. In some cases, the antibodies contain VH CDRs having the sequences set forth in SEQ ID NOs: 13 and 14. In some cases, the antibodies contain VH CDRs having the sequences set forth in SEQ ID NOs: 13 and 15. In some cases, the antibodies contain VH CDRs having the sequences set forth in SEQ ID NOs: 14 and 15. In some cases, the antibodies contain VH CDRs having the sequences set forth in SEQ ID NOs: 13, 14, and 15.

In some embodiments, the CDRs of the light chain of anti-MMP9 antibodies disclosed herein have the following amino acid sequences:

CDR1:
(SEQ ID NO: 16)
KASQDVRNTVA
CDR2:
(SEQ ID NO: 17)
SSSYRNT
CDR3:
(SEQ ID NO: 18)
QQHYITPYT

Thus, among the provided anti-MMP9 antibodies are antibodies having a light chain CDR1 region with an amino acid sequence as set forth in SEQ ID NO: 16, antibodies having a light chain CDR2 region with an amino acid sequence set forth in SEQ ID NO: 17, and antibodies having a light chain CDR3 region with an amino acid sequence as set forth in SEQ ID NO: 18, and antibodies that compete for binding with or bind to the same epitope on MMP9 as such antibodies. In some cases, the antibodies contain VL CDRs having the sequences set forth in SEQ ID NO: 18. In some cases, the antibodies contain VL CDRs having the sequences set forth in SEQ ID NOs: 16 and 17. In some cases, the antibodies contain VL CDRs having the sequences set forth in SEQ ID NOs: 16 and 18. In some cases, the antibodies contain VL CDRs having the sequences set forth in SEQ ID NOs: 17 and 18. In some cases, the antibodies contain VL CDRs having the sequences set forth in SEQ ID NOs: 16, 17, and 18.

An illustrative humanized variant anti-MMP9 antibody, AB0045 (humanized, modified IgG4 (S241P)) contains the humanized AB0041 heavy chain variant VH3 (having the sequence set forth in SEQ ID NO: 7 (QVQLQESGPGLVKPSETLSLTCTVSGFSLLSYGVHWVRQPPGKGLEWLGVIWTGGT TNYNSALMSRFTISKDDSKNTVYLKMNSLKTEDTAIYYCARYYYGMDYWGQGTLVT VSS) and the humanized AB0041 light chain variant Vk4 (having the light chain sequence set forth in SEQ ID NO: 12 (DIQMTQSPSSLSASVGDRVTITCKASQDVRNTVAWYQQKPGKAPKLLIYSSSYRNTG VP DRFSGSGSGTDFTLTISSLQAEDVAVYYCQQHYITPYTFGGGTKVEIK)).

The AB0045 antibody contains 1312 amino acids in length, is composed of two heavy chains and two light chains, and has a theoretical pI of about 7.90, extinction coefficient of about 1.50 AU/cm at 280 nm for 1 g/L, a molecular weight of about 144 kDa, and density of about 1 g/mL in formulation buffer (50-100 mg/mL product concentration).

The heavy chain of the AB0045 antibody has the sequence set forth in SEQ ID NO: 49 (MGWSLILLFLVAVATRVHSQVQLQESGPGLVKPSETLSLTCTVSGFSLLSYGVHWVR QPPGKGLEWLGVIWTGGTTNYNSALMSRFTISKDDSKNTVYLKMNSLKTEDTAIYYC ARYYYGMDYWGQGTLVTVSSASTKGPSVFPIAPCSRSTSESTAALGCLVKDYFPEPV TVSWNSGALTSGVHTFPAVLQSSGLYSLSSWTVPSSSLGTKTYTCNVDHKPSNTKVDKRVE SKYGPPCPPCPAPEFLGGPSVFLFPPKPKDTLMISRTPEVTCWVDVSQEDPEVQFNWYV DGVEVHNAKTKPREEQFNSTYRWSVLTVLHQDWLNGKEYKCKVSNKGLPSSIEKTISKAK GQPREPQVYTLPPSQEEMTKNQVSLTCLVKGFYPSDIAVEWESNGQPENNYKTTPPVLDS DGSFFLYSRLTVDKS RWQEGNVFSCSVMHEALHNHYTQKSLSLSLGK (signal sequence underlined; sequence of the constant region presented in italics)); the light chain of the AB0045 antibody has the sequence set forth in SEQ ID NO: 50 (MRVPAQLLGLLLLWLPGARCDIQMTQSPSSLSASVGDRVTITCKASQDVRNTVAWY QQKPGKAPKLLIYSSSYRNTGVPDRFSGSGSGTDFTLTISSLQAEDVAVYYCQQHYITP YTFGGGTKVEIKRTVAAPSVFIFPPSDEQLKSGTASVVCLLNNFYPREAKVQWKVDNALQ SGNSQESVTEQDSKDSTYSLSSTLTLSKADYEKHKVYACEVTHQGLSSPVTKSFNRGEC (signal sequence underlined; sequence of the constant region presented in italics).

The antibodies further include those produced by the hybridoma designated M4, i.e., an antibody containing the heavy chain (lgG2b) sequence: MAVLVLFLCLVAFPSCVLSQVQLKESGPGLVAPSQSLSITCTVSGFSLLSYGVHWVRQ PPGKGLEWLGVIWTGGSTNYNSALMSRLSISKDDSKSQVFLKMNSLQTDDTAMYYC ARYYYAMDYWGQGTSVTVSSAKTTPPSVYPIAPGCGDTTGSSVTLGCLVKGYFPES VTVTWNSGSLSSSVHTFPALLQSGLYTMSSSVTVPSSTWPSQTVTCSVAHPASSTTVDKKLEP SGPISTINPCPPCKECHKCPAPNLEGGPSVFIFPPNIKDVLMISLTPKVTCWVDVSEDDPD VRISWFVNNVEVHTAQTQTHREDYNSTIRVVSALPIQHQDWMSGKEFKCKVNNKDLPSPIE RTISKIKGLVRAPQVYILPPPAEQLSRKDVSLTCLVVGFNPGDISVEWTSNGHTEENYKDTA PVLDSDGSYFIYSKLD IKTSKWEKTDSFSCNVRHEGLKNYYLKKTISRSPGK (SEQ ID NO: 30) (signal peptide set forth in underlined text, variable region set forth in plain text, and constant region set forth in italics), and the light chain (kappa) sequence: MESQIQVFVFVFLWLSGVDGDIVMTQSHKFTSVGDRVSITCKASQDVRNTVAWY QQKTGQSPKLLIYSASYRNTGVPDRFTGSISGTDFTFTISSVQAEDLALYYCQQHYSTP YTFGGGTKLEVKRADAAPTVSIFPPSSEQLTSGGASWCFLNNFYPKDINVKWKIDGSER QNGVLNSWTDQDSKDSTYSMSSTLTLTKDEYERHNSYTCEATHKTSTSPIVKSFNRNEC (signal peptide set forth in underlined text, variable region set forth in plain text, and constant region set forth in italics) (SEQ ID NO: 31). The M4 antibody has a variable heavy chain with an amino acid sequence: QVQLKESGPGLVAPSQSLSITCTVSGFSLLSYGVHWVRQPPGKGLEWLGVIWTGGST NYNSALMSRLSISKDDSKSQVFLKMNSLQTDDTAMYYCARYYYAMDYWGQGTSVT VSS (CDRs 1, 2, and 3 (SEQ ID NOs: 34, 35, and 36, respectively) underlined) (SEQ ID NO: 32) and a variable light chain with the amino acid sequence DIVMTQSHKFMFTSVGDRVSITCKASQDVRNTVAWYQQKTGQSPKLLIYSASYRNTG VPDRFTGSISGTDFTFTISSVQAEDLALYYCQQHYSTPYTFGGGTKLEVK (CDRs 1, 2, and 3 (SEQ ID NOs: 37, 38, and 39, respectively) underlined) (SEQ ID NO: 33).

The M4 antibody heavy chain can have the amino acid sequence set forth in SEQ ID NO: 54: MAVLVLFLCLVAFPSCVLSQVQLKESGPGLVAPSQSLSITCTVSGFSLLSYGVHWVRQPP GKGLEWLGVIWTGGSTNYNSALMSRLSISKDDSKSQVFLKMNSLQTDDTAMYYCARY YYAMDYWGQGTSVTVSSAKTTPPSVYPLAPGCGDTTGSSVTLGCLVKGYFPESVTVTWNSG SL (signal peptide set forth in underlined text, variable region set forth in plain text, and a part of the constant region set forth in italics), and the M4 antibody light chain can have the amino acid sequence set forth in SEQ ID NO: 51: MESQIQVFVFVFLWLSGVDGDIVMTQSHKFMFTSVGDRVSITCKASQDVRNTVAWYQQ KTGQSPKLLIYSASYRNTGVPDRFTGSISGTDFTFTISSVQAEDLALYYCQQHYSTPYTFG GGTKLEVKRADAAPTVSIFPPSSEQLTSG (signal peptide set forth in underlined text, variable region set forth in plain text, and a part of the constant region set forth in italics).

The antibodies further include those produced by the hybridoma designated M12, i.e., one with only a kappa chain, having the sequence: QVFVYMLLWLSGVDGDIVMTQSQKFMSTSVGDRVSVTCKASQNVGTNVAWYQQKP GQSPKALIYSASYRFSGVPDRFTGSGSGTDFTLTISNVQSEDLAEYFCQQYNSYPYTFG GGTKLEIKRADAAPTVSIFPPSSEQLTSGGASWCFLNNFYPKDINVKWKIDGSERQNGVL NSWT DQDSKDSTYSMSSTLTLTKDEYERHNSYTCEATHKTSTSPIVKSFNRNEC (signal peptide set forth in underlined text, variable region set forth in plain text, and constant region set forth in italics) (SEQ ID NO: 40). The M12 antibody has a variable light chain with the amino acid sequence DIVMTQSQKFMSTSVGDRVSVTCKASQNVGTNVAWYQQKPGQSPKALIYSASYRFS GVPDRFTGSGSGTDFTLTISNVQSEDLAEYFCQQYNSYPYTFGGGTKLEIK (CDRs 1, 2, and 3 (SEQ ID NOs: 42, 43, and 44, respectively) underlined) (SEQ ID NO: 41).

The M12 antibody light chain can have the amino acid sequence set forth in SEQ ID NO: 53: QVFVYMLLWLSGVDGDIVMTQSQKFMSTSVGDRVSVTCKASQNVGTNVAWYQQKPG QSPKALIYSASYRFSGVPDRFTGSGSGTDFTLTISNVQSEDLAEYFCQQYNSYPYTFGGG TKLEIKRADAAPTVSIFPPSSEQLTSG (signal peptide set forth in underlined text, variable region set forth in plain text, and constant region set forth in italics).

The antibodies further include the mouse antibody designated AB0046, having a kappa light chain with an amino acid sequence MSSAQFLGLLLLCFQGTRCDIQMTQTTSSLSASLGDRVTISCSASQGISNYLNWYQQK PDGTFKLLIYYTSILHSGVPSRFSGSGSGTDYSLTISNLEPEDIATYYCQQYGWLPRTFG GGTKLEIKRADAAPTVSIFPPSSEQLTSGGASVVCFLNNFYPKDINVKWKIDGSERQNGVL NSWTDQDSKDSTYSMSSTLTLTKDEYERHNSYTCEATHKTSTSPIVKSFNRNEC (SEQ ID NO: 45) (signal peptide set forth in underlined text, variable region set forth in plain text, and constant region set forth in italics) and an IgG1 heavy chain with an amino acid sequence MGWSSIILFLVATATGVHSQVQLQQPGSVLVRPGASVKLSCTASGYTFTSYWMNWV KQRPGQGLEWIGEIYPISGRTNYNEKFKVKATLTVDTSSSTAYMDLNSLTSEDSAVYY CARSRANWDDYWGQGTTLTVSSAKTTPPSVYPLAPGSAAQTNSMVTLGCLVKGYFPE PVTVTWNSGSLSSGVHTFPAVLQSDLYTLSSSVTVPSSTWPSETVTCNVAHPASSTKVDKKIV PRDCGCKPCICTVPEVSSVFIFPPKPKDVLTITLTPKVTCVWDISKDDPEVQFSWFVDDVE VHTAQTQPREEQFNSTFRSVSELPIMHQDWLNGKEFKCRVNSAAFPAPIEKTISKTKGRPK APQVYTIPPPKEQ MAKDKVSLTCMITDFFPEDITVEWQWNGQPAENYKNTQPIMDTDGSYFVYSKLNVQKSN WE AGNTFTCSVLHEGLHNHHTEKSLSHSPGK (SEQ ID NO: 46) (signal peptide set forth in underlined text, variable region set forth in plain text, and constant region set forth in italics).

The following amino acid sequence comprises the framework regions and complementarity-determining regions (CDRs) of the variable region of the IgG1 heavy chain of AB0046 (with CDRs underlined):

(SEQ ID NO: 47)
QVQLQQPGSVLVRPGASVKLSCTASGYTFTSYWMNWVKQRPGQGLEWIG
EIYPISGRTNYNEKFKVKATLTVDTSSSTAYMDLNSLTSEDSAVYYCAR
SRANWDDYWGQGTTLTVSS.

The following amino acid sequence comprises the framework regions and complementarity-determining regions (CDRs) of the variable region of the kappa light chain of AB0046 (with CDRs underlined):

(SEQ ID NO: 48)
DIQMTQTTSSLSASLGDRVTISCSASQGISNYLNWYQQKPDGTFKLLIY
YTSILHSGVPSRFSGSGSGTDYSLTISNLEPEDIATYYCQQYGWLPRTF
GGGTKLEIK.

The antibodies for use with the presently provided methods, compositions, and combinations can include any of the antibodies described herein, including antibodies and antibody fragments, including those containing any combination of the various exemplified heavy and light chains, heavy and light chain variable regions, and CDRs. By way of example, the presently provided methods, compositions, and combinations comprise the antibody or antigen binding fragment thereof comprising an amino acid sequence of any of SEQ ID NOs: 5, 6, 7, 8, 9, 10, 11, 12, 13, 14, 15, 16, 17, 18, 30, 31, 32, 33, 34, 35, 36, 37, 38, 39, 40, 41, 42, 43, 44, 45, 46, 47, 48, 49, 50, 51, 53, or 54. Some embodiments of the methods, compositions, and combinations comprise the antibody or antigen binding fragment thereof comprising the amino acid sequences of SEQ ID NOs: 7 and 12. Certain embodiments of the methods, compositions, and combinations comprise the antibody or antigen binding fragment thereof comprising the amino acid sequences of SEQ ID NOs: 13, 14, 15, 16, 17, and 18.

In certain embodiments, an anti-MMP9 antibody is described in any of the following PCT applications: WO2012/027721, WO2013/130078, and WO2013/130905, herein incorporated by reference in their entireties.

In certain embodiments, an anti-MMP9 antibody is described in PCT Publication Nos. WO 2016/023979 or WO2016/023972, each of which is herein incorporated by reference in its entirety.

In certain embodiments, methods of the present disclosure may be practiced by providing to the subject one or more nucleic acid encoding any of the therapeutic agents described herein, e.g., a nucleic acid encoding an anti-MMP9 antibody or binding fragment thereof, thus providing to the subject the encoded polypeptide. In addition, compositions of the present disclosure include nucleic acids that encode any of the therapeutic agents described herein, e.g., mRNA or modified mRNA or expression vectors encoding a therapeutic polypeptide described herein. In various embodiments, the nucleic acid is single-stranded or double-stranded, RNA or DNA, e.g., mRNA or cDNA.

The present disclosure provides nucleic acids encoding anti-MMP9 antibodies and functional fragments thereof and any other polypeptide therapeutic agent described herein. Accordingly, the present disclosure provides an isolated polynucleotide (nucleic acid) encoding an antibody or antigen-binding fragment as described herein, vectors containing such polynucleotides, and host cells and expression systems for transcribing and translating such polynucleotides into polypeptides. In certain embodiments, the nucleic acids are single-stranded, double-stranded, RNA, mRNA, DNA, or cDNA, including modified forms thereof, e.g., comprising modifications to reduce immunogenicity or enhance stability.

The present disclosure contemplates constructs in the form of plasmids, vectors, transcription or expression cassettes which comprise at least one polynucleotide as above.

The present disclosure also provides a recombinant host cell which comprises one or more constructs as above, as well as methods of production of the antibody or antigen-binding fragments thereof described herein which method comprises expression of nucleic acid encoding a heavy chain polypeptide and a light chain polypeptide (in the same or different host cells, and from the same or different constructs) in a recombination host cell. Expression can be achieved by culturing under appropriate conditions recombinant host cells containing the nucleic acid. Following production by expression, an antibody or antigen-binding fragment can be isolated and/or purified using any suitable technique, then used as appropriate.

Systems for cloning and expression of a polypeptide in a variety of different host cells are well known. Suitable host cells include bacteria, mammalian cells, yeast and baculovirus systems. Mammalian cell lines available in the art for expression of a heterologous polypeptide include Chinese hamster ovary cells, HeLa cells, baby hamster kidney cells, NSO mouse melanoma cells and many others. A common bacterial host is E. coli.

Suitable vectors can be chosen or constructed, containing appropriate regulatory sequences, including operably linked promoter sequences, terminator sequences, polyadenylation sequences, enhancer sequences, marker genes and/or other sequences as appropriate. Vectors can be plasmids or viral, e.g., phage or phagemid, as appropriate. For further details see, for example, Molecular Cloning: a Laboratory Manual: 2nd edition, Sambrook et al., 1989, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Short Protocols in Molecular Biology, Second Edition, Ausubel et al. eds., John Wiley & Sons, 1992. The disclosures of Sambrook et al. and Ausubel et al. are incorporated herein by reference in their entirety.

The nucleic acid encoding a polypeptide of interest may be integrated into the genome of the host cell or can be maintained as a stable or transient episomal element.

Any of a wide variety of expression control sequences, i.e., sequences that control the expression of a DNA sequence operatively linked to it, can be used in these vectors to express the DNA sequences. For example, a nucleic acid encoding a polypeptide of interest can be operably linked to a promoter, and provided in an expression construct for use in methods of production of recombinant MMP9 proteins or portions thereof.

Those of skill in the art are aware that nucleic acids encoding the antibody chains disclosed herein can be synthesized using standard knowledge and procedures in molecular biology.

Examples of nucleotide sequences encoding the heavy and light chain amino acid sequences disclosed herein, are as follows:

VH1:
(SEQ ID NO: 19)
CAGGTGCAGC TGCAGGAATC CGGCCCTGGC CTGGTCAAGC
CCTCCGAGAC ACTGTCCCTG ACCTGCACCG TGTCCGGCTT
CTCCCTGCTG TCCTACGGCGTGCACTGGGTCCGACAGCCTCC
AGGGAAGGGCCTGGAATG GCTGGGCGTG ATCTGGACCG
GCGGCACCAC CAACTACAAC TCCGCCCTGA TGTCCCGGCT
GACCATCTCC AAGGACGACT CCAAGTCCAC CGTGTACCTG
AAGATGAACT CCCTGAAAAC CGAGGACACC GCCATCTACT
ACTGCGCCCG GTACTACTAC GGCATGGACT ACTGGGGCCA
GGGCACCTCC GTGACCGTGT CCTCA
VH2:
(SEQ ID NO: 20)
CAGGTGCAGC TGCAGGAATC CGGCCCTGGC CTGGTCAAGC
CCTCCGAGAC ACTGTCCCTG ACCTGCACCG TGTCCGGCTT
CTCCCTGCTG TCCTACGGCG TGCACTGGGT CCGACAGCCT
CCAGGCAAAG GCCTGGAATG GCTGGGCGTG ATCTGGACCG
GCGGCACCAC CAACTACAAC TCCGCCCTGA TGTCCCGGCT
GACCATCTCC AAGGACGACT CCAAGAACAC CGTGTACCTG
AAGATGAACT CCCTGAAAAC CGAGGACACC GCCATCTACT
ACTGCGCCCG GTACTACTAC GGCATGGACT ACTGGGGCCA
GGGCACCCTG GTCACCGTGT CCTCA
VH3:
(SEQ ID NO: 21)
CAGGTGCAGC TGCAGGAATC CGGCCCTGGC CTGGTCAAGC
CCTCCGAGAC ACTGTCCCTG ACCTGCACCG TGTCCGGCTT
CTCCCTGCTG TCCTACGGCG TGCACTGGGT CCGACAGCCT
CCAGGCAAAG GCCTGGAATG GCTGGGCGTG ATCTGGACCG
GCGGCACCAC CAACTACAAC TCCGCCCTGA TGTCCCGGTT
CACCATCTCC AAGGACGACT CCAAGAACAC CGTGTACCTG
AAGATGAACT CCCTGAAAAC CGAGGACACC GCCATCTACT
ACTGCGCCCG GTACTACTAC GGCATGGACT ACTGGGGCCA
GGGCACCCTG GTCACCGTGT CCTCA
VH4:
(SEQ ID NO: 22)
CAGGTGCAGCTGCAGGAATCCGGCCCTGGCCTGGTCAAGC
CCTCCGAGAC ACTGTCCCTG ACCTGCACCG TGTCCGGCTT
CTCCCTGCTG TCCTACGGCG TGCACTGGGT CCGACAGCCT
CCAGGCAAAG GCCTGGAATG GCTGGGCGTG ATCTGGACCG
GCGGCACCAC CAACTACAAC TCCGCCCTGA TGTCCCGGTT
CACCATCTCC AAGGACGACT CCAAGAACAC CCTGTACCTG
AAGATGAACT CCCTGAAAAC CGAGGACACC GCCATCTACT
ACTGCGCCCG GTACTACTAC GGCATGGACT ACTGGGGCCA
GGGCACCCTG GTCACCGTGT CCTCA
Vk1:
(SEQ ID NO: 23)
GACATCGTGA TGACCCAGTC CCCCAGCTTC CTGTCCGCCT
CCGTGGGCGA CAGAGTGACC ATCACATGCA AGGCCTCTCA
GGACGTGCGG AACACCGTGG CCTGGTATCA GCAGAAAACC
GGCAAGGCCC CCAAGCTGCT GATCTACTCC TCCTCCTACC
GGAACACCGG CGTGCCCGAC CGGTTTACCG GCTCTGGCTC
CGGCACCGAC TTTACCCTGA CCATCAGCTC CCTGCAGGCC
GAGGACGTGG CCGTGTACTT CTGCCAGCAG CACTACATCA
CCCCCTACAC CTTCGGCGGA GGCACCAAGG TGGAAATAAA
A
Vk2:
(SEQ ID NO: 24)
GACATCGTGA TGACCCAGTC CCCCTCCAGC CTGTCCGCCT
CTGTGGGCGA CAGAGTGACC ATCACATGCA AGGCCTCTCA
GGACGTGCGG AACACCGTGG CCTGGTATCA GCAGAAGCCC
GGCAAGGCCC CCAAGCTGCT GATCTACTCC TCCTCCTACC
GGAACACCGG CGTGCCCGAC CGGTTTACCG GCTCTGGCTC
CGGCACCGAC TTTACCCTGA CCATCAGCTC CCTGCAGGCC
GAGGACGTGG CCGTGTACTT CTGCCAGCAG CACTACATCA
CCCCCTACAC CTTCGGCGGA GGCACCAAGG TGGAAATAAA
A
Vk3:
(SEQ ID NO: 25)
GACATCCAGA TGACCCAGTC CCCCTCCAGC CTGTCCGCCT
CTGTGGGCGA CAGAGTGACC ATCACATGCA AGGCCTCCCA
GGACGTGCGG AACACCGTGG CCTGGTATCA GCAGAAGCCC
GGCAAGGCCC CCAAGCTGCT GATCTACTCC TCCTCCTACC
GGAACACCGG CGTGCCCGAC CGGTTCTCTG GCTCTGGAAG
CGGCACCGAC TTTACCCTGA CCATCAGCTC CCTGCAGGCC
GAGGACGTGG CCGTGTACTT CTGCCAGCAG CACTACATCA
CCCCCTACAC CTTCGGCGGA GGCACCAAGG TGGAAATAAA
A
Vk4:
(SEQ ID NO: 26)
GACATCCAGA TGACCCAGTC CCCCTCCAGC CTGTCCGCCT
CTGTGGGCGA CAGAGTGACC ATCACATGCA AGGCCTCTCA
GGACGTGCGG AACACCGTGG CCTGGTATCA GCAGAAGCCC
GGCAAGGCCC CCAAGCTGCT GATCTACTCC TCCTCCTACC
GGAACACCGG CGTGCCCGAC CGGTTCTCTG GCTCTGGAAG
CGGCACCGAC TTTACCCTGA CCATCAGCTC CCTGCAGGCC
GAGGACGTGG CCGTGTACTA CTGCCAGCAG CACTACATCA
CCCCCTACAC CTTCGGCGGA GGCACCAAGG TGGAAATAAA
A

Because the structure of antibodies, including the juxtaposition of CDRs and framework regions in the variable region, the structure of framework regions and the structure of heavy- and light-chain constant regions, is well-known in the art, it is well within the skill of the art to obtain related nucleic acids that encode anti-MMP9 antibodies. Accordingly, polynucleotides comprising nucleic acid sequences having at least 75%, at least 80%, at least 85%, at least 90%, at least 95%, at least 98% and at least 99% homology to any of the nucleotide sequences disclosed herein are also provided. Accordingly, polynucleotides comprising nucleic acid sequences having at least 75%, at least 80%, at least 85%, at least 90%, at least 95%, at least 98% and at least 99% identity to any of the nucleotide sequences disclosed herein are also provided. In one example, the polynucleotide contains at least at or about 75%, 80%, 85%, 90%, 91%, 92%, 93%, 94%, 95%, 96%, 97%, 98%, 99% or more sequence identity with SEQ ID NO: 21 or includes or is SEQ ID NO: 21 and/or contains at least at or about 75%, 80%, 85%, 90%, 91%, 92%, 93%, 94%, 95%, 96%, 97%, 98%, 99% or more sequence identity with SEQ ID NO: 26 or includes or is SEQ ID NO: 26.

### Methods

The compositions and methods of the present disclosure, such as MMP9 binding proteins and other therapeutic agents, e.g., TNFα inhibitors, chemotherapeutic agents, and immune checkpoint inhibitors, can be used, for example, for treating or preventing diseases and conditions, e.g., pathological conditions. In certain embodiments, the disease or condition is selected from myeloid cell-associated inflammation; cystic fibrosis, non-cystic fibrosis bronchiectasis, sarcoidosis, idiopathic pulmonary fibrosis, tuberculosis, a cancer, an autoimmune or inflammatory disease or condition, vasculitis, septicemia, multiple sclerosis, muscular dystrophy, lupus, allergy, asthma, and hidradenitis suppurativa. In certain embodiments, the diseases and conditions include cystic fibrosis, cancer, autoimmune diseases or conditions, or inflammatory diseases or conditions. Thus, in one embodiment, the application provides therapeutic methods and uses of the anti-MMP9 antibodies, alone or in combination with one or more additional therapeutic agents, e.g., a chemotherapeutic agent, an anti-cancer agent, an anti-angiogenic agent, an anti-fibrotic agent, an immunomodulating agent, an immunotherapeutic agent, an immune modulating agent, a therapeutic antibody, a radiotherapeutic agent, an anti-neoplastic agent, an anti-proliferation agent, or any combination thereof.

Provided herein are methods for treating or preventing a disease or disorder, comprising providing to the subject: an effective amount of an Matrix Metalloproteinase 9 (MMP9) binding protein; and, optionally, an effective amount of one or more additional therapeutic agent, thereby treating or preventing the disease or condition in the subject. Examples of MMP9 binding agents and other therapeutic agents that may be used according to the methods described herein are provided herein. In certain embodiments, an MMP9 binding protein comprises an immunoglobulin heavy chain polypeptide, or functional fragment thereof, and an immunoglobulin light chain polypeptide, or functional fragment thereof, wherein the MMP9 binding protein specifically binds MMP9. In some embodiments, the MMP9 binding protein and/or the additional therapeutic agent is selected from the group consisting of an antibody, a small molecule and a recombinant molecule.

Also provided is use of: a Matrix Metalloproteinase 9 (MMP9) binding protein; and optionally, one or more additional therapeutic agents, in the manufacture of a medicament for the treatment or prevention of a disease or condition. Examples of MMP9 binding agents and other therapeutic agents that may be used according to the methods described herein are provided herein. In certain embodiments, an MMP9 binding protein comprises an immunoglobulin heavy chain polypeptide, or functional fragment thereof, and an immunoglobulin light chain polypeptide, or functional fragment thereof, wherein the MMP9 binding protein specifically binds MMP9.

As demonstrated in the Examples, expression of matrix metalloproteinases (MMPs) and MMP9 in particular is associated with a variety of disease pathologies, including autoimmune diseases or conditions, inflammatory diseases or conditions, and oncology. MMP9 can promote disease through its destructive remodeling of basement membrane and other structural proteins, and/or by increasing vascular permeability and bioavailability of growth factors and cytokines such as TGF, VEGF, TNFα, IL-6, and IL-1. MMP9 regulates the bioavailability of ECM-sequestered VEGF and FGF-2, as well as membrane-tethered EGF. As described in the Examples, specific inhibition of MMP9, using antibodies as described herein, was efficacious in accepted mouse models of cancer and inflammatory diseases, such as vasculitis, breast cancer and colorectal cancer. Furthermore, the combination of an anti-MMP9 antibody and a TNFα inhibitor was effective at ameliorating disease in a mouse model of rheumatoid arthritis.

Also provided are pharmaceutical compositions for use in connection with such methods, such as those containing any of the MMP9 binding proteins, antibodies or fragments thereof described herein, alone or in combination with one or more additional therapeutic agent. Compositions can be suitable for administration locally or systemically by any suitable route.

In general, therapeutic agents of the present disclosure are provided to a subject in a therapeutically effective amount. In some embodiments, a therapeutic agent is provided to a subject in an amount to effect inhibition of MMP9 activity, to inhibit TNFα, to inhibit immune checkpoint mediators, or to treat myeloid cell-associated inflammation. In some embodiments, the disease or condition is: cystic fibrosis; non-cystic fibrosis bronchiectasis; sarcoidosis; idiopathic pulmonary fibrosis; tuberculosis; a cancer, optionally selected from the group consisting of pancreatic cancer, esophagogastric adenocarcinoma, non-small cell lung cancer, lung squamous cell carcinoma, lung adenocarcinoma, gastric adenocarcinoma, colorectal carcinoma, pancreatic adenocarcinoma, head and neck squamous cell carcinoma, hepatocellular carcinoma, colorectal cancer, colorectal adenocarcinoma and hepatocellular carcinoma; an autoimmune or inflammatory disease or condition, optionally selected from the group consisting of rheumatoid arthritis, an inflammatory bowel disease (IBD), vasculitis (optionally large vessel vasculitis (e.g., Takayasu arteritis and Giant cell arteritis), medium vessel vasculitis (e.g., Polyarteritis Nodosa and Kawasaki Disease), immune complex small vessel vasculitis (e.g., Cryoglobulinemic vasculitis, IgA vasculitis (Henoch-Schonlein), and hypocomplementemic urticarial vasculitis (anti-C1q vasculitis)), anti-GBM Disease, ANCA-associated small vessel vasculitis (e.g., microscopic polyangiitis, granulomatosis with polyangiitis (Wegner's), and eosinophilic granulomatosis with polyangiitis (Churg-Strauss)), septicemia; multiple sclerosis; muscular dystrophy; lupus; allergy; asthma or hidradenitis suppurativa; or an inflammatory bowel disease, optionally selected from the group consisting of: ulcerative colitis (UC), Crohn's disease (CD), or indeterminate colitis. In another embodiment, the autoimmune or inflammatory disease or condition is rheumatoid arthritis, an inflammatory bowel disease (IBD), septicemia, multiple sclerosis, muscular dystrophy, lupus, allergy or asthma. In a further embodiment, the inflammatory bowel disease (IBD) is ulcerative colitis (UC), Crohn's disease (CD), or indeterminate colitis.

In certain embodiments, each therapeutic agent of the present disclosure (e.g., an antibody that binds MMP9 or a functional fragment thereof) is provided to a subject at the interval of one, two or three weeks, or once every one, two, or three weeks. In certain embodiments, each therapeutic agent can be provided daily or less frequently than daily, for example, six times a week, five times a week, four times a week, three times a week, twice a week, once a week, once every two weeks, once every three weeks, once a month, once every two months, once every three months, or once every six months. In some embodiments, the treatment includes at least one, at least two, at least three, at least four, at least five, at least six, at least seven, at least eight, at least nine, or at least ten administration(s). The compositions may also be administered in a sustained release formulation, such as in an implant which gradually releases the composition for use over a period of time, and which allows for the composition to be administered less frequently, such as once a month, once every 2-6 months, once every year, or even a single administration. Also, the treatment is continuous. In one embodiment, each therapeutic agent, the composition or the formulation thereof is provided once a week. In certain embodiments, each therapeutic agent, the composition or the formulation thereof is provided once every two weeks. In some embodiments, each therapeutic agent is provided at different frequencies. In one embodiment, the antibody that binds MMP9 or a functional fragment thereof is administered once a week, while the TNFα inhibitor is administered once a month. In another embodiment, the antibody that binds MMP9 or a functional fragment thereof is administered once a week, while the immune checkpoint inhibitor is administered once a month.

Each therapeutic agent of the present disclosure (e.g., an antibody that binds MMP9 or a functional fragment thereof) can be administered to an individual via any route, including, but not limited to, intravenous (e.g., by infusion pumps), intraperitoneal, intra-arterial, intrapulmonary, oral, inhalation, intravesicular, intramuscular, intra-tracheal, subcutaneous, intrathecal, transdermal, transpleural, topical, inhalational (e.g., as mists of sprays), mucosal (such as via nasal mucosa), subcutaneous, transdermal, gastrointestinal, intraarticular, intracisternal, or intraventricular. In some embodiments, the compositions are administered systemically (for example by intravenous injection). In some embodiments, each therapeutic agent is administered locally (for example by intra-arterial or injection). In some embodiments, each therapeutic agent is administered subcutaneously. In some embodiments, each therapeutic agent is administered intradermally. In some embodiments, each therapeutic agent is administered via inhalation. In some embodiments, each therapeutic agent is administered mucosally. In one embodiment, each therapeutic agent, the composition or the formulation thereof is delivered by intravenous administration (i.e. intravenous infusion) twice every two weeks. In certain embodiments, each therapeutic agent, the composition or the formulation thereof is delivered by subcutaneous administration once every week. In some embodiments, each therapeutic agent is administered via different routes. In one embodiment, the antibody that binds MMP9 or a functional fragment thereof is administered subcutaneously, while the TNFα inhibitor is administered subcutaneously or intravenously. In another embodiment, the antibody that binds MMP9 or a functional fragment thereof is administered subcutaneously, while the immune checkpoint inhibitor is administered subcutaneously or intravenously.

In some embodiments, each therapeutic agent of the present disclosure (e.g., an antibody that binds MMP9 or a functional fragment thereof) is administered at about 25 mg per subject to about 800 mg per subject or at the recommended dosage for the particular therapeutic agent. In some embodiments, each therapeutic agent is administered at about 50 mg, about 100 mg, about 200 mg, about 300 mg, about 400 mg, about 500 mg, about 600 mg, about 700 mg, or about 800 mg per subject, including any range in between these values. In certain embodiments, each therapeutic agent is administered at about 150 mg, about 250 mg, about 350 mg, about 450 mg, about 550 mg, about 650 mg, or about 750 mg per subject, including any range in between these values. In some embodiments, each therapeutic agent of the above dosage is administered once a week, once every two weeks, once every three weeks, once a month, once every two months, once every three months, or once every six months. In some embodiments, each therapeutic agent is administered at about 400 mg every two weeks. In certain embodiments, each therapeutic agent is administered to the subject at a dosage of about 200 mg every two weeks. In certain embodiments, each therapeutic agent is administered at about 150 mg once a week. In certain embodiments, each therapeutic agent is administered at about 300 mg once a week. In certain embodiments, each therapeutic agent is administered to the subject in a two-step procedure: first, a loading dose phase (more frequent dosing to cover the “target sink”/“tissue and serum sink” or high baseline concentration of MMP9 associated with the disease, wherein the dosing range is administered to the subject at a dosage of about 200 mg, about 300 mg, or about 400 mg every week for an interval of one, two or three weeks, or more frequent dosing to cover the “target sink” or high baseline concentration of MMP9 associated with the disease) and second, once a predictable pK has been established after the loading dose phase, a lower weekly dose such as 150, 125, 100 or 50 mg/week. In some embodiments, the lower weekly dose could be lower on a weekly basis, e.g., 150, 125, 100 or 50 mg/week. In one embodiment, each therapeutic agent, the composition or the formulation thereof is administered intravenously (i.e. intravenous infusion) at about 400 mg every two weeks. In one embodiment, each therapeutic agent, the composition or the formulation thereof is administered intravenously at about 200 mg every two weeks. In one embodiment, each therapeutic agent, the composition or the formulation thereof is administered subcutaneously (i.e. subcutaneous injection) at about 150 mg once a week. In one embodiment, each therapeutic agent, the composition or the formulation thereof is administered subcutaneously at about 300 mg every two weeks. In some embodiments, each therapeutic agent is administered at a dose, frequency and route that are distinct from the dose, frequency and route of another therapeutic agent.

The selected dosage regimen will depend upon a variety of factors including the activity of the therapeutic agent, the route of administration, the time of administration, the rate of excretion of the particular compound being employed, the duration of the treatment, other drugs, compounds and/or materials used in combination with the particular composition employed, the age, sex, weight, condition, general health and prior medical history of the patient being treated, and like factors well known in the medical arts.

In some embodiments, dosage is determined based on a pharmacokinetic model for antibodies displaying target-mediated disposition. In contrast to the relatively linear pharmacokinetics observed for antibodies directed to soluble receptor targets, antibodies directed toward tissue-based target receptors frequently demonstrate non-linear pharmacokinetics. Mager, D. E. (2006), Adv Drug Deliv Rev 58(12-13): 1326-1356. The basis for non-linear disposition relates to the high affinity binding of antibody to target and the extent of binding (relative to dose), such that the interaction is reflected in the pharmacokinetic characteristics of the antibody. Mager, D. E. and W. J. Jusko (2001), J Pharmacokinet Pharmacodyn 28(6): 507-532. Included within target mediated drug disposition is receptor-mediated endocytosis (internalization) of the antibody-receptor complex. Wang, W., E. Q. Wang, et al. (2008), Clin Pharmacal Ther 84(5): 548-558.

In a pharmacokinetic model for an antibody having target-mediated disposition, in the absence of drug (antibody), the target receptor is synthesized at a constant rate and eliminated by a first-order process. As a result, the target receptor exists at a steady-state concentration in the absence of drug (antibody). When drug is added to the body it can interact with the target receptor in a bimolecular reaction, distribute into less well perfused tissue, or be eliminated via first-order processes. At low drug concentrations the predominant movement of drug is onto the receptor due to the high affinity binding. As the amount of drug entering the body becomes sufficient to bind the available mass of receptor the drug distributes into and out of tissue and is eliminated. As drug concentrations fall and drug equilibrates from tissue this provides an additional reservoir to binding newly synthesized receptor.

A clinician having ordinary skill in the art can readily determine and prescribe the effective amount (ED50) of the pharmaceutical composition required. For example, the physician or veterinarian can start doses of the compounds of the disclosure employed in the pharmaceutical composition at levels lower than that required in order to achieve the desired therapeutic effect and gradually increase the dosage until the desired effect is achieved.

In some cases, the methods of treatment include parenteral administration, e.g., intravenous, intra-arterial, intradermal, intramuscular, or subcutaneous administration, or oral administration of the agent, e.g., anti-MMP9 antibody or composition containing the same; TNFα inhibitor or composition containing the same; immune checkpoint inhibitor or composition containing the same.

In some embodiments, the subject treated has been diagnosed with, is diagnosed with, or is considered at risk of developing a disease or condition, e.g., cystic fibrosis; a cancer, optionally selected from the group consisting of pancreatic cancer, esophagogastric adenocarcinoma, non-small cell lung cancer, lung squamous cell carcinoma, lung adenocarcinoma, gastric adenocarcinoma, colorectal carcinoma, pancreatic adenocarcinoma, head and neck squamous cell carcinoma, hepatocellular carcinoma, colorectal cancer, colorectal adenocarcinoma and hepatocellular carcinoma; an autoimmune or inflammatory disease or condition, optionally selected from the group consisting of rheumatoid arthritis, an inflammatory bowel disease (IBD), vasculitis (optionally large vessel vasculitis (e.g., Takayasu arteritis and Giant cell arteritis), medium vessel vasculitis (e.g., Polyarteritis Nodosa and Kawasaki Disease), immune complex small vessel vasculitis (e.g., Cryoglobulinemic vasculitis, IgA vasculitis (Henoch-Schonlein), and hypocomplementemic urticarial vasculitis (anti-C1q vasculitis)), anti-GBM Disease, ANCA-associated small vessel vasculitis (e.g., microscopic polyangiitis, granulomatosis with polyangiitis (Wegner's), and eosinophilic granulomatosis with polyangiitis (Churg-Strauss)), septicemia, multiple sclerosis, muscular dystrophy, lupus, allergy, asthma or hidradenitis suppurativa; or an inflammatory bowel disease, optionally selected from the group consisting of: ulcerative colitis (UC), Crohn's disease (CD), or indeterminate colitis. In another embodiment, the autoimmune or inflammatory disease or condition is rheumatoid arthritis, an inflammatory bowel disease (IBD), septicemia, multiple sclerosis, muscular dystrophy, lupus, allergy or asthma. In a further embodiment, the inflammatory bowel disease (IBD) is ulcerative colitis (UC), Crohn's disease (CD), or indeterminate colitis. In certain embodiments, the subject is a human having cystic fibrosis, a cancer, an inflammatory disease or condition, or an autoimmune disease or condition, and can be treated as described herein. In certain embodiments, the subject is a human.

In certain embodiments, the subject or diseased cells of the subject overexpress MMP9, e.g., express at least 1.2-fold, at least 1.5-fold, at least 2-fold, at least 3-fold, at least 5-fold, or at least 10-fold higher amounts of MMPs than a control subject or non-diseased cells.

In certain embodiments, any of the methods described herein further comprises determining an amount of MMP9, e.g., active MMP9, present in the subject or tissue or cells therefrom, e.g., diseased tissue or cells obtained from the subject, and comparing the amount to a control amount, such as a predetermined control value or an amount determined from a normal subject or normal tissue or cells. In certain embodiments, the subject is provided with the MMP9 binding protein and immune modulatory agent if the amount of MMP9 determined for the subject is higher than the control amount, e.g., at least 1.2-fold, at least 1.5-fold, at least 2-fold, at least 3-fold, or at least 5-fold higher than the control amount, but is not treated if the amount of MMP9 determined for the subject is not higher than the control value.

In some embodiments, the antibody, e.g., AB0045, is used in treating patients having advanced pancreatic or esophagogastric adenocarcinoma, non-small cell lung cancer, ulcerative colitis, colorectal cancer, Crohn's disease, or rheumatoid arthritis. In some aspects of such embodiments, the patients are administered the anti-MMP9 antibody or antigen binding fragment thereof intravenously at a dosage of 100, 200, 300, 400, 500, 600, 700, 800, 900, 1000, 1100, 1200, 1300, 1400, 1500, 1600, 1700, or 1800 mg, at the interval of one, two or three weeks. In some aspects, the appropriate dosage is made with 0.9% sodium chloride. In some aspects, the patients receive the antibody, e.g., AB0045, as monotherapy or as part of a combination therapy with other therapeutic agents.

In some embodiments, for pancreatic adenocarcinoma, the anti-MMP9 antibody or antigen binding fragment thereof is administered alone at the two-week interval or with the 28-day cycle chemotherapy of gemcitabine and/or nab-paclitaxel.

In some embodiments, for esophagogastric adenocarcinoma, the anti-MMP9 antibody or antigen binding fragment thereof is administered alone at the two-week interval or with the 28-day cycle chemotherapy of mFOLFOX6 that is administered in a 28-day cycle.

In some embodiments, for non-small cell lung cancer, the anti-MMP9 antibody or antigen binding fragment thereof is administered alone at the three-week interval or with the 21-day cycle chemotherapy of carboplatin and paclitaxel or with pemetrexed and/or bevacizumab.

In one example, for colorectal cancer, the anti-MMP9 antibody or antigen binding fragment thereof is administered alone at a two-week interval or with a 14-day cycle chemotherapy of FOLFIRI. In some aspects of the combination treatments, the chemotherapy or immunotherapy agent is administered with the known dosage and procedure.

In some aspects, the dosage of MMP9 antibody can be adjusted and administered at about 133, about 267, about 400, about 600 or about 1200 mg. After each therapeutic cycle, the patients are monitored for the levels of MMP9 antibodies, MMP9, or other suitable biomarkers.

In some embodiments, the treatment methods include steps for monitoring treatment, including for monitoring efficacy or activity, such as pharmacodynamic activity. In some examples, such methods include detecting or measuring the presence, absence, levels, and/or expression of markers, such as cytokines and other inflammatory markers that are indicative of efficacy of treatment, in biological test samples obtained from subjects being treated using the methods and compositions. The samples typically are blood samples or serum samples but can include other biological samples as described herein. Among the markers for use in such methods are Tissue Inhibitor of Metalloproteinases 1 (TIMP-1), Tumor Necrosis Factor alpha (TNF-alpha), Macrophage Inflammatory Protein-2 (MIP-2), Interleukin-17A (IL-17A), CXCL1O, Lymphotactin, Macrophage Inflammatory Protein-1 beta (MIP-1 beta), Oncostatin-M (OSM), Interleukin-6 (IL-6), Monocyte Chemotactic Protein 3 (MCP-3), Vascular Endothelial Growth Factor A (VEGF-A), Monocyte Chemotactic Protein-5 (MCP-5), Interleukin-1 alpha (IL-1 alpha), Macrophage Colony-Stimulating Factor-1 (M-CSF-1), Myeloperoxidase (MPO), Growth-Regulated Alpha Protein (KC/GRO), Interleukin-7 (IL-7), Leukemia Inhibitory Factor (LIP), Apolipoprotein A-I (Apo A-I), C-Reactive Protein (CRP), Granulocyte Chemotactic Protein-2 (GCP-2), Interleukin-11 (IL-11), Monocyte Chemotactic Protein 1 (MCP-1), von Willebrand factor (vWF), and Stem Cell Factor (SCF) gene products. In some embodiments, the markers are selected from among KC/GRO, LIP, CXCL1O, MPO, MIP-2, and MCP-5 gene products, for example, when the diseases is IBD, such as UC.

In some embodiments, after each therapeutic cycle, the patients are monitored for the levels of MMP9 antibodies, MMP9, or other suitable biomarkers.

Among the provided methods are those that provide improved safety profiles compared to available treatments and therapeutic regimens and/or sustained long-term efficacy in treating such diseases and conditions.

### Diseases and Conditions

Compositions, methods and kits described herein are used to treat a variety of diseases and conditions, e.g., pathological conditions, including but not limited to any of those described herein.

In certain embodiments, any of the compositions and methods described herein are used to treat or prevent a disease or condition, e.g., a disease or condition associated with MMP9. An MMP9-associated disease or condition includes a disease or condition where MMP9 expression or activity is deregulated and/or where the disease or condition can be treated or prevented with one or more modulators of MMP9, such as an MMP9 binding protein comprising an immunoglobulin heavy chain polypeptide, or functional fragment thereof, and an immunoglobulin light chain polypeptide, or functional fragment thereof, wherein the MMP9 binding protein specifically binds MMP9, optionally in combination with one or more additional therapeutic agent. In one embodiment, the disease or condition is associated with an increase in total MMP9 protein in the subject or diseased cells, as compared to a normal control. In yet another embodiment, the MMP9-associated disease or condition is associated with an increase in, or elevated levels of, active MMP9 protein in the subject having the disease or disorder or diseased cells therefrom, as compared to a normal control. As described in the Examples, high levels of active MMP9 or total MMP9 are detected in tissues from patients suffering from diseases such as ulcerative colitis, Crohn's disease, vasculitis, and cystic fibrosis, or in an animal model of colorectal cancer. In certain embodiments, the MMP9-associated disease or disorder is associated with a level of active MMP9 protein at least 1.1-fold, at least 1.2-fold, at least 1.5-fold, at least 2-fold, at least 3-fold, or at least 5-fold the level of active MMP9 protein in a normal control subject or normal control cells. In certain embodiments, a normal control subject is a subject not diagnosed with or having the disease or condition, and normal control cells are non-diseased cells of the same type as the diseased cells of the subject.

In one embodiment, the MMP9-associated disease or condition comprises myeloid cell-associated inflammation. In some embodiments, the MMP9-associated disease or condition is: cystic fibrosis, a cancer, or an autoimmune or inflammatory disease or condition. In certain embodiments, the cancer is selected from the group consisting of: pancreatic cancer, esophagogastric adenocarcinoma, non-small cell lung cancer, lung squamous cell carcinoma, lung adenocarcinoma, gastric adenocarcinoma, colorectal carcinoma, pancreatic adenocarcinoma, head and neck squamous cell carcinoma, hepatocellular carcinoma, colorectal cancer, colorectal adenocarcinoma and hepatocellular carcinoma. In certain embodiments, the autoimmune or inflammatory disease or condition is selected from rheumatoid arthritis, an inflammatory bowel disease (IBD), vasculitis, septicemia, multiple sclerosis, muscular dystrophy, lupus, allergy, asthma and hidradenitis suppurativa. In certain embodiments, the inflammatory bowel disease is selected from the group consisting of: ulcerative colitis (UC), Crohn's disease (CD), or indeterminate colitis. In other embodiments, the autoimmune or inflammatory disease or condition is rheumatoid arthritis, an inflammatory bowel disease (IBD), septicemia, multiple sclerosis, muscular dystrophy, lupus, allergy or asthma. In a further embodiment, the inflammatory bowel disease (IBD) is ulcerative colitis (UC), Crohn's disease (CD), or indeterminate colitis. In yet another embodiment, the vasculitis is large vessel vasculitis (e.g., Takayasu arteritis and Giant cell arteritis), medium vessel vasculitis (e.g., Polyarteritis Nodosa and Kawasaki Disease), immune complex small vessel vasculitis (e.g., Cryoglobulinemic vasculitis, IgA vasculitis (Henoch-Schonlein), and hypocomplementemic urticarial vasculitis (anti-C1q vasculitis)), anti-GBM Disease, ANCA-associated small vessel vasculitis (e.g., microscopic polyangiitis, granulomatosis with polyangiitis (Wegner's), or eosinophilic granulomatosis with polyangiitis (Churg-Strauss).

In some embodiments, the methods and compositions described herein, e.g., antibodies and fragments thereof, are used in the treatment of inflammatory and autoimmune disease, e.g., by inhibiting MMP9 in subjects having such diseases or conditions. Among the inflammatory and autoimmune diseases are inflammatory bowel disease (IBD) (including Crohn's disease, ulcerative colitis (UC), and indeterminate colitis), collagenous colitis, rheumatoid arthritis, septicemia, multiple sclerosis, muscular dystrophy, lupus, allergy, septicemia, and asthma.

As described in the Examples, MMP9 and other MMPs are involved in inflammatory and autoimmune diseases. Matrix metalloproteinase-9 (MMP9) is induced in the serum, synovial fluid, and synovium of RA patients, and the MMP9/TIMP-1 ratio is altered in favor of increased proteolytic activity. MMP9 is secreted by disease-mediating osteoclasts and activated cells of the monocyte/macrophage lineage. Resistance to antibody-induced arthritis disease phenotypes is observed in a MMP9 knock-out mouse strain. MMP9 degrades the unwound collagen II created by the cleavage activity of collagenases, such as MMP8, and thereby contributes to the destruction of articular cartilage.

As shown in the Examples herein, anti-MMP9 antibodies were effective in various inflammatory and autoimmune diseases, including vasculitis and rheumatoid arthritis (RA) in animal models. Thus, in some embodiments, the methods, compositions, and kits described herein are used to treat subjects having inflammatory and autoimmune diseases. In some embodiments, the methods, compositions, and kits are used to treat subjects having a cancer. In some embodiments, the inhibitors, methods, and kits are used to inhibit MMP9 without inhibiting other MMPs, such as without inhibiting MMP2, or without inhibiting such other MMPs to a substantial degree. In one embodiment, the methods protect against or reduce tissue injury, systemic inflammation, and/or local inflammation in a subject having such a disease or condition; in some examples, both tissue injury and inflammation are treated by the methods. In another embodiment, the methods are associated with reduced toxicity and/or reduced induction of musculoskeletal syndrome (MSS) or similar symptoms, compared to that observed with pan-MMP inhibitors, such as Marimastat. In some examples, the subject has had an inadequate response to another therapy for the inflammatory disease, such as a TNF-antagonist, such as an anti-TNF antibody, e.g., infliximab, i.e., has TNF-antagonistic refractive disease. Thus, among the provided methods are those effective at treating inflammation in such subjects. Illustrative, non-limiting disease and disorders that may be treated or prevented using composition and methods of the present disclosure are described.

Inflammatory Bowel Disease

Inflammatory bowel diseases (IBDs) include but are not limited to Crohn's disease, ulcerative colitis (UC), and indeterminate colitis). Ulcerative colitis (UC) is one of the two major IBDs, characterized by diffuse mucosal inflammation, and associated ulceration, of the colon. The chronic course of UC includes intermittent disease exacerbations followed by periods of remission. Many patients experience insufficient response to agents such as anti-TNFα targeted therapeutics and continue to suffer from disease-related symptoms. Patients with UC have a significantly elevated risk of colon cancer after 8-10 years of disease activity.

Inflammatory bowel disease (IBD) therapeutics can modulate disease by preventing recruitment and access of inflammatory cells to the disease site, preventing activation of cells at the disease site, and/or inhibiting the downstream effects of cell activation.

UC pharmacologic treatment generally proceeds ‘by line’ based on disease severity and the location or extent of the disease. Disease severity is characterized as mild, moderate or severe based on patient symptoms, endoscopic findings, and laboratory results and in the clinical trial setting often defined by the Mayo Score, as shown in Table 1B.

TABLE 1B
UC Mayo Score

| Subscore | Definition |
|---|---|
| Stool Frequency | |
| 0 | Normal for the patient |
| 1 | 1-2 stools more than normal |
| 2 | 3-4 stools more than normal |
| 3 | ≧5 stools more than normal |
| Rectal Bleeding | |
| 0 | No blood seen |
| 1 | Streaks of blood with stool less than half |
| | of the time |
| 2 | Obvious blood with stool most of the time |
| 3 | Blood alone passes |
| Findings on Endoscopy | |
| 0 | Normal or inactive disease |
| 1 | Mild disease (erythema, decreased |
| | vascular pattern, mild friability) |
| 2 | Moderate disease (marked erythema, lack |
| | of vascular pattern, friability, erosions) |
| 3 | Severe disease (spontaneous bleeding |
| | ulceration) |
| Physician's global assessment | |
| 0 | Normal |
| 1 | Mild disease |
| 2 | Moderate disease |
| 3 | Severe disease |

As described in the Examples, evidence supports a role for MMP9 in the pathology of ulcerative colitis (UC) and other inflammatory bowel diseases (IBDs). Broad-spectrum MMP inhibitors are efficacious in TNBS and DSS models of colitis (Naito and Yoshikawa 2005; Medina and Radomski 2006). While MMP9 and MMP2 are the two most closely related MMPs, with similar substrate specificities, MMP9 protein and activity are induced to a greater extent in IBD and preclinical colitis animal models and more strongly induced and associated with progressive disease in human UC; MMP2 is more ubiquitously expressed and plays is important for homeostasis of non-diseased tissue. Lack of MMP9 protects against colitis in the mouse dextran sodium sulfate (DSS)-induced model, while MMP2 serves a protective function for the colon. Neutrophil and lymphocyte accumulation in the DSS model is MMP9-dependent; there is evidence for epithelial cell-derived MMP9 contribution to tissue damage.

MMP9 was detected in human UC tissues, not in healthy colonic crypts (in which the distinct ring of collagen IV staining marked intact basement membranes), but in areas of disorganized collagen IV, which indicates loss of basement membrane integrity. MMP9 degrades collagen IV and other ECM components, allowing infiltration of inflammatory cells. In colitis, MMP9 activity in the mucosa can lead to degradation of the basement membranes underlying crypts, and mucosal damage and exposure of the submucosa to luminal bacteria. MMP9 degradation of the basement membrane around blood vessels can promote extravasation of leukocytes to the disease site. MMP9 activity in the extracellular matrix can activate and release inflammatory cytokines such as TNFα, IL-6, and IL1-B that contribute to disease progression.

Available UC therapies have not been entirely satisfactory. For example, different treatments generally are given based on severity, location and/or extent of disease. For less severe disease, treatments include 5′-aminosalicylate (5′-ASA) enemas, corticosteroid enemas and oral 5′-ASA preparations. Patients with more severe disease, and/or those failing to respond to first line therapies are generally treated with a course of oral corticosteroids. Immunomodulators such as azathioprine and 6-mercaptopurine (6-MP) are used to help wean subjects off steroids and to maintain remission. Anti-TNFα therapy, e.g., the chimeric antibody Remicade® (infliximab) is generally used in patients with more severe disease and for patients who are refractory to or dependent upon corticosteroids. Infliximab treatment generally fails to induce and maintain steroid-free remission over the long term. Only 20% of patients achieve a remission by week 8 and remain in remission through 54 weeks, with the majority of patients relapsing by week 30. Only 26% of patients were able to achieve a long-term remission completely free of corticosteroids. When the less stringent endpoint of response is evaluated instead of remission (indicating an incomplete reduction in symptoms), approximately 60% of patients fail to maintain this degree of relief over 30 or 54 weeks. Thus it may be beneficial to use anti-MMP9 antibodies or antigen binding fragments thereof as an add-on therapy with TNFα inhibitors for patients who still have disease despite receiving anti-TNFα therapy.

Cyclosporine has helped delay the need for surgery in patients hospitalized for fulminant UC, but its efficacy as a maintenance therapy has not been established. Surgery, consisting of a two-step total colectomy with ileal pouch anal anastomosis (IPAA) is curative. A total colectomy is, however, is an undesirable outcome for many patients, committing them to lifelong frequent bowel movements, a high risk of sexual dysfunction, and a 50% risk of developing pouchitis—an inflamed J pouch that results in diarrhea with or without rectal bleeding, tenesmus, urgency, pain, incontinence and fevers. Furthermore, the risk of female infertility is highly increased following IPAA surgery.

As shown in WO 2013/130905, which is herein incorporated in its entirety, specific anti-MMP9 antibodies were demonstrated as effective in an accepted UC animal model, effectively protecting against tissue destruction and aberrant tissue remodeling, as well as local and systemic downregulation of pro-inflammatory factors. The antibodies had robust efficacy on multiple endpoints in treatment of DSS-induced colitis in mice, a well-established preclinical model used for evaluation of agents being considered for treatment of UC. Thus, in some embodiments, the methods and compositions are used to treat a subject with an inflammatory bowel disease, such as ulcerative colitis (UC), Crohn's disease, or indeterminate colitis. In some embodiments, the methods and antibodies inhibit the MMP9 without inhibiting other MMPs, such as MMP2.

In some examples, the methods and compositions protect against destruction of basement membrane, mucosal damage, exposure of submucosa to luminal bacteria, inflammation, cytokine activation and leukocyte extravasation. In some embodiments, the subject has moderate to severe UC, e.g., has severe UC. In some embodiments, the subject has steroid dependent UC. In some aspects, the treatment methods replace or are administered as an alternative to corticosteroid treatment.

In some embodiments, the subject treated has been non-responsive to other UC therapies, such as TNF (e.g., TNF-alpha or TNF-α) antagonists, such as anti-TNF antibodies (such as infliximab and/or adalimumab), i.e., TNF antagonist-refractory patients. For example, in some embodiments, the subject is a patient who has failed to achieve long-term remission on infliximab therapy or other TNF-alpha targeting treatment. In other cases, the subject has been non-responsive to another UC therapy such as oral or rectal application treatments such as enemas, suppositories and foam), 5-aminosalicylic acid (5-ASAs), oral and rectal application corticosteroids, immunosuppressants such as 6-mercaptopurine, azathioprine, methotrexate, and/or cyclosporine. In some aspects, the methods provide treatment with an improved safety protocol as compared to such treatments, or provide treatment with more sustained, long-term efficacy. In some embodiments, the subject is treated with a combination of an anti-MMP9 therapeutic and an anti-TNFα therapeutic.

In some cases, the methods inhibit MMP9 without affecting other MMPs, such as MMP2.

In some embodiments, in the context of UC, “response” to treatment is achieved if there is at least a 3 point and a 30% reduction in the Mayo Score with at least a 1 point reduction in the rectal bleeding subscore or an absolute rectal bleeding subscore of 0-1. In some embodiments, “remission” is defined as a Mayo score≦2, with no individual subscore>1. In some embodiments, “mucosal healing” is defined as an endoscopic subscore to ≦1. In some embodiments, “steroid sparing” is defined as remission in the absence of ongoing steroid use for those patients who began on steroids. In some embodiments, quality of life is an endpoint and is assessed using known methods, such as a validated quality of life measure such as the IBD-QoL or the SF-36.

Crohn's disease (CD) is a chronic inflammatory disorder of the gastrointestinal tract defined by relapsing and remitting episodes, with progression to complications such as fistula formation, abscesses, or strictures. Extraintestinal manifestations such as uveitis, arthritis, skin lesions, and kidney stones occur in upwards of 40% of patients. The treatment paradigm for mild-to-moderate Crohn's has been antibiotics such as ciprofloxacin and flagyl, 5-ASAs, budesonide, or systemic corticosteroids, however, the long-term side effects of systemic steroids greatly dampens their utility. Patients with mild-to-moderate disease who fail these first line therapies are often placed on the on azathioprine remain in remission at one-year. For patients who fail azathioprine or those with more severe disease, TNF-α blockade with agents such as infliximab remain the last option. As opposed to UC where surgical resection is curative, such therapy is more difficult for Crohn's patients for two reasons: 1) disease is diffuse throughout the GI tract and in instances of isolated disease (e.g., terminal ileum), resection is frequently associated with recurrent disease at the site of the resection 2) since the disease is transmural, surgical resection places patients at risk for future stricture and/or fistula development.

While combination therapy using azathioprine and infliximab may be superior to either therapy alone for induction of remission and mucosal healing at 26 weeks, the concurrent use of such agents increases the risk of infection and malignancy (hepatosplenic T cell lymphoma), limiting their utility. As with UC, response, remission, mucosal healing, steroid sparing and quality of life will all be important endpoints, but in CD the Crohn's Disease Activity Index (CDAI) is generally the validated outcome instrument of choice and is described in Table 1C:

TABLE 1C
Crohn's Disease Activity Index:

| METRIC | VALUE | FORMULA |
|---|---|---|
| Liquid stools | Daily total × 7 days | Total Sum × 2 |
| Abdominal Pain | Daily total × 7 days | Sum × 5 |
| | NONE = 0 | |
| | Intermediate = 1 | |
| | Severe = 3 | |
| General well being | Daily total × 7 days | Sum × 7 |
| | Well = 0 | |
| | Intermediate = 1, 2, | |
| | 3 | |
| Extra-intestinal | One point for each: | Score × 20 |
| | Arthritis/arthralgia | |
| | Iritis/uveitis | |
| | Skin/mouth ulcers | |
| | Peri-anal disease | |
| | Other fistula | |
| | Fever >37.8 C. | |
| Anti-diarrheal use | YES/NO | Value × 30 |
| Abdominal Mass | None = 0 | Value × 10 |
| | Questionable = 2 | |
| Hematocrit (Hct) | Males: 47-Hct | Value × 6 |
| | Females: 42-Hct | |

Weight | OCCASIONALLY USED
Score <150 = Remission
Moderate Disease ≧220
Severe disease ≧450
Response to therapy = decrease of greater than 70 or alternatively 100 point decrease can be used to define response.

In some embodiments, the subject has moderate to severe CD, e.g., has severe CD. In some embodiments, the subject has steroid dependent CD. In some aspects, the treatment methods replace or are administered as an alternative to corticosteroid treatment.

In some embodiments, the subject has been non-responsive to other CD therapies, such as TNF antagonists, such as anti-TNF antibodies (such as infliximab and/or adalimumab), i.e., TNF antagonist-refractory patients. For example, in some embodiments, the subject is a patient who has failed to achieve long-term remission on infliximab therapy or other TNF-alpha targeting treatment. In other cases, the subject has been non-responsive to another CD therapy. In some aspects, the methods provide treatment with an improved safety protocol as compared to such treatments, or provide treatment with more sustained, long-term efficacy. In some embodiments, the subject suffering from Crohn's is treated with a combination of an anti-MMP9 therapeutic and an anti-TNFα therapeutic.

Rheumatoid Arthritis

Rheumatoid arthritis (RA) is a chronic, systemic inflammatory disease that affects approximately 1.3 million adults in the United States (US). Rheumatoid arthritis manifests principally as an attack on peripheral joints and may lead to marked destruction and deformity of joints, with considerable disability and impact on quality of life. It is characterized by the production of autoantibodies, synovial inflammation with formation of pannus tissue, and erosion of underlying cartilage and bone. Although people of any age can be affected, the onset of RA is most frequent between the ages of 40 and 50 years, and women are affected 3 times more often than men. While the cause of RA is still not completely understood, aberrant B-cell activation, T-cell co-stimulation, osteoclast differentiation, and cytokine release all have been implicated in its pathogenesis. Patients with RA experience a high risk of disability and mortality.

Despite recent advances in RA treatment, including tumor necrosis factor alpha (TNFα) targeted therapeutics, a number of patients experience insufficient response to these agents and continue to suffer from disease-related symptoms, as well as incurring joint damage. MMP9 has been reported to play an important role in the progression of RA, and is known to be expressed in human RA as well as animal models of disease. The role of MMP9 in disease progression in RA is supported by findings in the MMP9 knockout mouse, which is significantly protected against increased disease severity in a collagen-induced arthritis model of RA, whereas matrix metalloproteinase 2 (MMP2) knockout mice develop more severe disease than littermate controls. Tartrate resistant acid phosphatase (TRAP) positive mononuclear and multinucleated cells are often found in the synovium at the sites of cartilage and bone destruction. TRAP-positive multinucleated cells from RA patients, including osteoclasts, secrete MMP9 and are key participants in joint destruction. Furthermore, MMP9 has been shown to play a critical role in osteoclast invasion. Studies in a variety of different disease models and correlations in human disease support a role for MMP9 in driving inflammation through increased vascular permeability and through promoting the activation or increasing the bioavailability of cytokines and growth factors. Selective inhibition of MMP9 has the potential to slow and/or halt progression of bone and joint erosion, as well as to reduce inflammation.

Cystic Fibrosis

Cystic fibrosis (CF) affects approximately 100,000 people worldwide. CF is the most common life-shortening genetic disorder in Caucasians, with a median age of death of 27.5 years in the US and 28.0 years in the EU. CF is an autosomal recessive disorder characterized by progressive, obstructive pulmonary disease. Patients with CF are particularly susceptible to chronic airway infections with opportunistic bacteria such as Staphylococcus aureus, Haemophilus influenzae, Pseudomonas aeruginosa (PA), Stenotrophomonas maltophilia, Achromobacter species, and Burkholderia species.

The majority (70%) of patients with CF die of cardiorespiratory failure. This is the end result of a continuous cycle of airway obstruction, inflammation, and infection leading to bronchiectasis, parenchymal destruction, and loss of pulmonary function. Patients also experience episodes of acute pulmonary exacerbation, which is characterized by worsening respiratory symptoms and an acute decline in lung function.

The current standard of care in CF includes treatment with inhaled anti-pseudomonal antibiotics (eg, tobramycin and Cayston®) and mucolytics (eg, dornase). Recently, two CF transmembrane conductance regulator (CFTR) modulator therapies have been approved for treatment of CF patients with select genetic mutations. Ivacaftor (Kalydeco®) has been approved for CF patients who carry one of the G551D CFTR gating mutations. The other is a combination product combining ivacaftor with lumacaftor for CF patients who are homozygous with the most common CF mutation, F508del. Both drugs improve lung function and reduced pulmonary exacerbations. While current CTFR modulator therapies provide clinical benefit to almost 50% of the CF population, the therapy does not represent a complete clinical cure.

In certain embodiments, methods are provided for treating or preventing cystic fibrosis, comprising providing to the subject an effective amount of an MMP9 binding protein, e.g., an MMP9 binding protein comprising an immunoglobulin heavy chain polypeptide, or functional fragment thereof, and an immunoglobulin light chain polypeptide, or functional fragment thereof, wherein the MMP9 binding protein specifically binds MMP9, thereby treating or preventing cystic fibrosis in the subject. In one embodiment, the cystic fibrosis comprises myeloid cell-associated inflammation. In some embodiments, the anti-MMP9 antibody or antigen binding fragment thereof prevents cleavage of α1-antitrypsin to an inactive form.

Idiopathic Pulmonary Fibrosis

Inflammation and fibrosis underlies many lung diseases from cystic fibrosis to COPD to other interstitial lung diseases (ILDs). Therefore, modification of the cellular microenvironment could provide broad benefit to a number of lung disease patients.

One such ILD, idiopathic pulmonary fibrosis (IPF), is a grievous interstitial lung disease that is associated with a median survival of 2-3 years from initial diagnosis (King, T. E., Jr. et al. Idiopathic pulmonary fibrosis. Lancet (2011) 378(9807): 1949-1961; Rafii, R. et al. A review of current and novel therapies for idiopathic pulmonary fibrosis. J Thorac Dis (2013) 5(1): 48-73). It is characterized by fibrotic scarring of the lung and progressive loss of lung function. Two drugs, pirfenidone and nintedanib, have been approved for treatment of IPF in the United States on the basis that they slow the rate of disease progression, as measured by the rate of FVC decline over 1 year (Kreuter, M. et al. Pharmacological Treatment of Idiopathic Pulmonary Fibrosis: Current Approaches, Unsolved Issues, and Future Perspectives. Biomed Res Int (2015) 2015: 329481; Noble, P. W. et al. Pirfenidone for idiopathic pulmonary fibrosis: analysis of pooled data from three multinational phase 3 trials. Eur Respir J (2016) 47(1): 243-253; Richeldi, L. et al. Nintedanib in patients with idiopathic pulmonary fibrosis: Combined evidence from the TOMORROW and INPULSIS trials. Respir Med 2016). However, the improvements in pulmonary function seen with these treatments have not yet translated into improvements in mortality risk or cure (Canestaro W. et al. Drug Therapy for Treatment of Idiopathic Pulmonary Fibrosis: Systematic Review and Network Meta-Analysis. Chest 2016). Therefore, IPF remains a high unmet medical need.

Vasculitis and Giant Cell Arteritis

Vasculitis is inflammation of blood vessel walls. It causes changes in the walls of blood vessels, including thickening, weakening, narrowing and scarring. These changes restrict blood flow, resulting in organ and tissue damage. There are many types of vasculitis (see below), and most of them are rare. Vasculitis might affect just one organ, such as the skin, or it may involve several organs. The condition can be acute or chronic. Vasculitis can affect anyone, though some types are more common among certain groups. Certain patients can improve without treatment, while others will need medications to control the inflammation and prevent flare-ups. Vasculitis is also known as angiitis and arteritis.

Giant cell arteritis (GCA) is an auto-inflammatory/auto-immune disease that targets life-sustaining tissues, specifically the aorta and its major branches. Abnormal immune response driven by T cells and macrophages lead to destruction of the vessel wall and induce maladaptive repair mechanisms that eventually cause vessel occlusion and resulting organ ischemia. Affected patients are at high risk for suffering ischemic optic neuropathy, CNS ischemia, aortic arch syndrome and often have disabling systemic inflammation and muscle pain (polymyalgia rheumatic). There is currently no approved medication beyond corticosteroids which can be used for induction purposes, to treat freshly diagnosed cases, or for maintenance therapy. Steroids are highly effective in suppressing IL-6 in GCA, but are only treating one part of the disease process. Therefore, alternative treatment approaches are needed to prevent the progressive deterioration of arterial function and avoid ischemic complications.

Using gene expression profiling in temporal artery lesions of GCA patients, MMP9 transcript are one of the most abundant observed. This observation has been confirmed by immunohistochemistry, which indicates strong immunoreactivity to macrophages localized to fragmented internal elastic membrane suggesting a pathogenic function in this particular form of vasculitis. The topographical distribution of biologically active MMPs was also assessed using in-situ gelatinase zymography. Fully-developed lesions harbored the highest level of enzymatic activity when compared to biopsies with adventitial involvement or control arteries. The density of inflammatory infiltrates was found to be related to gelatinase activity. Vascular smooth muscle cells have also been reported to express MMP9. Furthermore, MMP9 serum level was found to be significantly higher in untreated GCA patients compared to healthy controls. Macrophage infiltrates in GCA are believed to be essential for sustaining adaptive T cell responses in addition to forming multi-nucleated giant cells. In the inflamed vessel wall niche of GCA patients, a large proportion of macrophages secrete PDGF, which trigger migratory and proliferative signaling pathways in VSMCs. More importantly, experimental systems have shown that PDGF promotes the migration of smooth muscle cells by inducing MMP9 and that the level of tissue-derived PDGF has been positively associated with the degree of intimal hyperplasia and angiogenesis. On a similar note, VEGF production by giant cells and macrophages is considered to be essential for the neovasculogenic process often observed in vasculitis and is also a potent inducer of MMP9 expression in T-lymphocytes and VSMCs. Finally, MMP9 is a limiting factor in the process of granuloma formation, the pathologic hallmark of GCA. In light of these findings, MMP9 activity is likely to be a central driver of arterial stenosis in patients diagnosed with GCA and is therefore an ideal drug target for experimental therapies.

Vasculitides can be categorized by the type of vessels involved. Large vessel vasculitis (LVV) include Takayasu arteritis (TAK), giant cell arteritis (GCA); Medium vessel vasculitis (MVV) include polyarteritis nodosa (PAN) and Kawasaki disease (KD); Small vessel vasculitis (SVV) include antineutrophil cytoplasmic antibody (ANCA)-associated vasculitis (AAV), microscopic polyangiitis (MPA), granulomatosis with polyangiitis (Wegener's) (GPA), eosinophilic granulomatosis with polyangiitis (Churg-Strauss) (EGPA), immune complex SVV, Anti-glomerular basement membrane (anti-GBM) disease, cryoglobulinemic vasculitis (CV), IgA vasculitis (Henoch-Schonlein) (IgAV), Hypocomplementemic urticarial vasculitis (HUV) (anti-C1q vasculitis); Variable vessel vasculitis (VVV) include Behcet's disease (BD) and Cogan's syndrome (CS); Single-organ vasculitis (SOV) include cutaneous leukocytoclastic angiitis, cutaneous arteritis, primary central nervous system vasculitis, isolated aortitis, among others; Vasculitis associated with systemic disease include lupus vasculitis rheumatoid vasculitis, sarcoid vasculitis, among others; Vasculitis associated with probable etiology include Hepatitis C virus-associated cryoglobulinemic vasculitis, Hepatitis B virus-associated vasculitis, syphilis-associated aortitis, drug-associated immune complex vasculitis, drug-associated ANCA-associated vasculitis, cancer-associated vasculitis, among others. In certain embodiments, compositions and methods described here treat or prevent any type of vasculitis.

In certain embodiments, methods are provided for treating or preventing vasculitis, comprising providing to the subject an effective amount of an MMP9 binding protein, e.g., an MMP9 binding protein comprising an immunoglobulin heavy chain polypeptide, or functional fragment thereof, and an immunoglobulin light chain polypeptide, or functional fragment thereof, wherein the MMP9 binding protein specifically binds MMP9, thereby treating or preventing vasculitis in the subject. In one embodiment, the vasculitis comprises myeloid cell-associated inflammation. In yet another embodiment, the vasculitis is giant cell arteritis.

Hidradenitis Suppurativa

Hidradenitis suppurativa is a chronic skin condition that features pea-sized to marble-sized lumps under the skin. Also known as acne inversa, these deep-seated lumps typically develop where skin rubs together—such as the armpits, groin, between the buttocks and under the breasts. The lumps associated with hidradenitis suppurativa are usually painful and may break open and drain foul-smelling pus. In many cases, tunnels connecting the lumps will form under the skin. Hidradenitis suppurativa tends to start after puberty, persist for years and worsen over time. Early diagnosis and treatment of hidradenitis suppurativa can help manage the symptoms and prevent new lesions from developing.

Cancer

In some embodiments, the methods and compositions, e.g., antibodies and fragments thereof, are used in the treatment of cancers and tumors and associated diseases and conditions. Cancers and tumors that may be treated as described herein include but are not limited to pancreatic cancer, esophagogastric adenocarcinoma, non-small cell lung cancer, lung squamous cell carcinoma, lung adenocarcinoma, gastric adenocarcinoma, colorectal carcinoma, pancreatic adenocarcinoma, head and neck squamous cell carcinoma, breast cancer, hepatocellular carcinoma, colorectal cancer, colorectal adenocarcinoma and hepatocellular carcinoma. Illustrative cancers include colorectal cancers, gastric adenocarcinoma, colorectal adenocarcinoma, and hepatocellular carcinoma.

Gastric Adenocarcinoma

Adenocarcinoma of the stomach is the most common gastrointestinal cancer in the world and the third leading cause of cancer death worldwide (Ferlay J, Soerjomataram I, Ervik M, Dikshit R, Eser S, Mathers C, et al. GLOBOCAN 2012 v1.0, Cancer Incidence and Mortality Worldwide: IARC CancerBase No. 11. Available at globocan.iarc.fr. Accessed 9 Jul. 2014. International Agency for Research on Cancer 2013). Approximately 22,220 patients are diagnosed annually in the United States, of whom 10,990 are expected to die. While the incidence of distal gastric adenocarcinoma has recently declined in the United States, gastric adenocarcinoma remains quite frequent in certain minority populations and it is still the second most common cause of cancer death worldwide. In addition, adenocarcinoma of the gastroesophageal junction (GEJ) is one of the most rapidly increasing solid tumors in the United States and Western Europe.

Most patients with gastric adenocarcinoma in the United States are symptomatic and already have advanced incurable disease at the time of presentation. At diagnosis, approximately 50 percent have disease that extends beyond locoregional confines, and only one-half of those who appear to have locoregional tumor involvement can undergo a potentially curative resection. Surgically curable early gastric adenocarcinomas are usually asymptomatic and only infrequently detected outside the realm of a screening program. Screening is not widely performed, except in countries which have a very high incidence, such as Japan, Venezuela, and Chile. The common presenting symptoms and diagnostic approaches to gastric adenocarcinoma include weight loss (usually results from insufficient caloric intake rather than increased catabolism) and may be attributable to anorexia, nausea, abdominal pain, early satiety, and/or dysphagia. Abdominal pain is often present which tends to be epigastric, vague, and mild early in the disease but more severe and constant as the disease progresses. Dysphagia is a common presenting symptom in patients with cancers arising in the proximal stomach or at the esophagogastric junction. Patients may also present with nausea or early satiety from the tumor mass or in cases of an aggressive form of diffuse-type gastric adenocarcinoma called linitis plastica, from poor distensibility of the stomach. They may also present with a gastric outlet obstruction from an advanced distal tumor.

Gastric and esophageal adenocarcinomas are chemotherapy sensitive diseases, with several active drug therapy classes, including platinum, fluoropyrimidines, topoisomerases inhibitors, taxanes, and anthracyclines. Despite significant differences in epidemiology and molecular characteristics, cytotoxic chemotherapy combinations have not demonstrated significant differences in efficacy across gastric adenocarcinoma (Chau I, Norman A R, Cunningham D, Oates J, Hawkins R, Iveson T, et al. The impact of primary tumour origins in patients with advanced oesophageal, oesophago-gastric junction and gastric adenocarcinoma—individual patient data from 1775 patients in four randomised controlled trials. Ann Oncol 2009; 20 (5):885-91).

Chemotherapy clearly provides a survival advantage over best supportive care in both first-line and second-line settings (Glimelius B, Ekstrom K, Hoffman K, Graf W, Sjoden P O, Haglund U, et al. Randomized comparison between chemotherapy plus best supportive care with best supportive care in advanced gastric cancer. Ann Oncol 1997; 8 (2):163-8; Murad A M, Santiago F F, Petroianu A, Rocha P R, Rodrigues M A, Rausch M. Modified therapy with 5-fluorouracil, doxorubicin, and methotrexate in advanced gastric cancer. Cancer 1993; 72 (1):37-41; Pyrhonen S, Kuitunen T, Nyandoto P, Kouri M. Randomised comparison of fluorouracil, epidoxorubicin and methotrexate (FEMTX) plus supportive care with supportive care alone in patients with non-resectable gastric cancer. Br J Cancer 1995; 71 (3):587-91; Scheithauer W, Komek G, Zeh B, Stoger F X, Schenk T, Henja M, et al. Palliative Chemotherapy vs. Supportive Care in Patients With Metastatic Gastric Cancer: A Randomized Trial [Abstract 68]. Conference on Biology, Prevention and Treatment of Gastrointestinal Malignancies; 1995 09-12 January; Koln, Germany; Kang J H, Lee S I, Lim do H, Park K W, Oh S Y, Kwon H C, et al. Salvage chemotherapy for pretreated gastric cancer: a randomized phase III trial comparing chemotherapy plus best supportive care with best supportive care alone. J Clin Oncol 2012; 30 (13):1513-8; Thuss-Patience P C, Kretzschmar A, Deist T, Hinke A, Bichev D, Lebedinzew B, et al. Irinotecan versus best supportive care (BSC) as second-line therapy in gastric cancer: A randomized phase III study of the Arbeitsgemeinschaft Internistische Onkologie (AIO) [Abstract 4540]. J Clin Oncol (ASCO Annual Meeting Abstracts) 2009). A meta-analysis of first-line chemotherapy versus best supportive case studies reported a hazard ration (HR) of 0.39 (95% CI, 0.28-0.52; p<0.001) for overall survival in favor of chemotherapy. This translates to a benefit of a median of 6 months (Wagner A D, Grothe W, Haerting J, Kleber G, Grothey A, Fleig W E. Chemotherapy in advanced gastric cancer: a systematic review and meta-analysis based on aggregate data. J Clin Oncol 2006; 24 (18):2903-9).

Performance status often declines after first-line therapy. Patients with esophageal cancer often have significant comorbidities, including obesity, heart disease, emphysema, which when coupled with progressive dysphagia and malnutrition, often limit therapeutic opportunities after first-line therapy. Gastric adenocarcinoma patients who develop peritoneal carcinomatosis often have decreased bowel function that then results in GI symptoms and a decline in functional status and therefore limiting treatment options substantially (Power D G, Kelsen D P, Shah M A. Advanced gastric cancer—slow but steady progress. Cancer treatment reviews 2010; 36 (5):384-92). However, administration of second-line therapy in patients who are sufficiently fit to receive it has demonstrated a survival advantage over supportive care alone (Kang et al. 2012; Thuss-Patience et al. 2009). A meta-analysis of these studies demonstrated a HR for overall survival of 0.73 (95% CI, 058-0.960), and in highly functioning patients (ECOG performance status of 0 or 1) the HR was 0.57 (95% CI 0.36-0.91).

For patients who retain an adequate performance status, there is no standard approach for second-line therapy after failure of the first-line regimen. Quality-of-life and minimization of side effects are key considerations when choosing the therapeutic approach. The choice of regimen is empiric. No single regimen has emerged as clearly superior and few trials have compared different regimens (Wesolowski R, Lee C, Kim R. Is there a role for second-line chemotherapy in advanced gastric cancer? Lancet Oncol 2009; 10 (9):903-12; Thallinger C M, Raderer M, Hejna M. Esophageal cancer: a critical evaluation of systemic second-line therapy. J Clin Oncol 2011; 29 (35):4709-14).

### Therapeutic Agents

Certain embodiments of the present application include or use one or more additional therapeutic agent. The one or more additional therapeutic agent may be an agent useful for the treatment of cancer, inflammation, autoimmune disease and related conditions. The one or more additional therapeutic agent may be a chemotherapeutic agent, an anti-angiogenic agent, an anti-fibrotic agent, an anti-inflammatory agent, an immune modulating agent, an immunotherapeutic agent, a therapeutic antibody, a radiotherapeutic agent, an anti-neoplastic agent or an anti-cancer agent, an anti-proliferation agent, or any combination thereof. In some embodiments, the MMP9 binding proteins described herein may be used or combined with a chemotherapeutic agent, an anti-angiogenic agent, an anti-fibrotic agent, an anti-inflammatory agent, an immune modulating agent, an immunotherapeutic agent, a therapeutic antibody, a radiotherapeutic agent, an anti-neoplastic agent or an anti-cancer agent, an anti-proliferation agent, or any combination thereof. In certain embodiments, an MMP9 binding protein described herein may be used or combined with an anti-neoplastic agent or an anti-cancer agent, anti-fibrotic agent, an anti-anti-inflammatory agent, or an immune modulating agent. In some embodiment, an MMP9 binding protein described herein may be used or combined with an anti-neoplastic agent or an anti-cancer agent. In certain embodiments, an MMP9 binding protein described herein may be used or combined with an immune modulating agent. In certain other embodiments, an MMP9 binding protein described herein may be used or combined with an anti-inflammatory agent. These therapeutic agents may be in the forms of compounds, antibodies, polypeptides, or polynucleotides.

In some embodiments, the application provides pharmaceutical compositions comprising an MMP9 binding protein and/or one or more additional therapeutic agent, and a pharmaceutically acceptable diluent, carrier or excipient. In one embodiment, the pharmaceutical compositions comprise an MMP9 binding protein, one or more additional therapeutic agent, and a pharmaceutically acceptable excipient, carrier or diluent. In some embodiments, the pharmaceutical compositions comprise the anti-MMP9 antibody AB0045. In some embodiments, the pharmaceutical compositions comprise a chemotherapeutic agent, an anti-angiogenic agent, an anti-fibrotic agent, an anti-inflammatory agent, an immune modulating agent, an immunotherapeutic agent, a therapeutic antibody, a radiotherapeutic agent, an anti-neoplastic agent or an anti-cancer agent, an anti-proliferation agent, or any combination thereof. In certain embodiments, the pharmaceutical compositions comprise the anti-MMP9 antibody AB0045, at least one additional therapeutic agent that is an immunomodulating agent, and a pharmaceutically acceptable diluent, carrier or excipient. In certain other embodiments, the pharmaceutical compositions comprise the anti-MMP9 antibody AB0045, at least one additional therapeutic agent that is an anti-inflammatory agent, and a pharmaceutically acceptable diluent, carrier or excipient. In certain other embodiments, the pharmaceutical compositions comprise the anti-MMP9 antibody AB0045, at least one additional therapeutic agent that is an anti-neoplastic agent or anti-cancer agent, and a pharmaceutically acceptable diluent, carrier or excipient.

In certain embodiments, the one or more additional therapeutic agent is an immune modulating agent, e.g., an immunostimulant or an immunosuppressant. In certain other embodiments, an immune modulating agent is an agent capable of altering the function of immune checkpoints, including the CTLA-4, LAG-3, B7-H3, B7-H4, Tim3, BTLA, KIR, A2aR, CD200 and/or PD-1 pathways. In other embodiments, the immune modulating agent is immune checkpoint modulating agents. Exemplary immune checkpoint modulating agents include anti-CTLA-4 antibody (e.g., ipilimumab), anti-LAG-3 antibody, anti-B7-H3 antibody, anti-B7-H4 antibody, anti-Tim3 antibody, anti-BTLA antibody, anti-KIR antibody, anti-A2aR antibody, anti CD200 antibody, anti-PD-1 antibody, anti-PD-L1 antibody, anti-CD28 antibody, anti-CD80 or -CD86 antibody, anti-B7RP1 antibody, anti-B7-H3 antibody, anti-HVEM antibody, anti-CD137 or -CD137L antibody, anti-OX40 or -OX40L antibody, anti-CD40 or -CD40L antibody, anti-GAL9 antibody, anti-IL-10 antibody and A2aR drug. For certain such immune pathway gene products, the use of either antagonists or agonists of such gene products is contemplated, as are small molecule modulators of such gene products. In certain embodiments, the immune modulatory agent is an anti-PD-1 or anti-PD-L1 antibody. In some embodiments, immune modulating agents include those agents capable of altering the function of mediators in cytokine mediated signaling pathways.

In certain embodiments, one or more additional therapeutic agent is an immune checkpoint inhibitor. Tumors subvert the immune system by taking advantage of a mechanism known as T-cell exhaustion, which results from chronic exposure to antigens and is characterized by the up-regulation of inhibitory receptors. These inhibitory receptors serve as immune checkpoints in order to prevent uncontrolled immune reactions.

PD-1 and co-inhibitory receptors such as cytotoxic T-lymphocyte antigen 4 (CTLA-4, B and T Lymphocyte Attenuator (BTLA; CD272), T cell Immunoglobulin and Mucin domain-3 (Tim-3), Lymphocyte Activation Gene-3 (Lag-3; CD223), and others are often referred to as a checkpoint regulators. They act as molecular determinants to influence whether cell cycle progression and other intracellular signaling processes should proceed based upon extracellular information.

In addition to specific antigen recognition through the T-cell receptor (TCR), T-cell activation is regulated through a balance of positive and negative signals provided by co-stimulatory receptors. These surface proteins are typically members of either the TNF receptor or B7 superfamilies. Agonistic antibodies directed against activating co-stimulatory molecules and blocking antibodies against negative co-stimulatory molecules may enhance T-cell stimulation to promote tumor destruction.

Programmed Cell Death Protein 1, (PD-1 or CD279), a 55-kD type 1 transmembrane protein, is a member of the CD28 family of T cell co-stimulatory receptors that include immunoglobulin superfamily member CD28, CTLA-4, inducible co-stimulator (ICOS), and BTLA. PD-1 is highly expressed on activated T cells and B cells. PD-1 expression can also be detected on memory T-cell subsets with variable levels of expression. Two ligands specific for PD-1 have been identified: programmed death-ligand 1 (PD-L1, also known as B7-H1 or CD274) and PD-L2 (also known as B7-DC or CD273). PD-L1 and PD-L2 have been shown to down-regulate T cell activation upon binding to PD-1 in both mouse and human systems (Okazaki et al., Int Immunol., 2007; 19: 813-824). The interaction of PD-1 with its ligands, PD-L1 and PD-L2, which are expressed on antigen-presenting cells (APCs) and dendritic cells (DCs), transmits negative regulatory stimuli to down-modulate the activated T cell immune response. Blockade of PD-1 suppresses this negative signal and amplifies T cell responses. Numerous studies indicate that the cancer microenvironment manipulates the PD-L1/PD-1 signaling pathway and that induction of PD-L1 expression is associated with inhibition of immune responses against cancer, thus permitting cancer progression and metastasis. The PD-L1/PD-1 signaling pathway is a primary mechanism of cancer immune evasion for several reasons. This pathway is involved in negative regulation of immune responses of activated T effector cells found in the periphery. PD-L1 is up-regulated in cancer microenvironments, while PD-1 is also up-regulated on activated tumor infiltrating T cells, thus possibly potentiating a vicious cycle of inhibition. This pathway is also intricately involved in both innate and adaptive immune regulation through bi-directional signaling. These factors make the PD-1/PD-L1 complex a central point through which cancer can manipulate immune responses and promote its own progression.

The first immune-checkpoint inhibitor to be tested in a clinical trial was ipilimumab (Yervoy, Bristol-Myers Squibb), an CTLA-4 mAb. CTLA-4 belongs to the immunoglobulin superfamily of receptors, which also includes PD-1, BTLA, TIM-3, and V-domain immunoglobulin suppressor of T cell activation (VISTA). Anti-CTLA-4 mAb is a powerful checkpoint inhibitor which removes “the break” from both naive and antigen-experienced cells. Therapy enhances the antitumor function of CD8+ T cells, increases the ratio of CD8+ T cells to Foxp3+T regulatory cells, and inhibits the suppressive function of T regulatory cells. TIM-3 has been identified as another important inhibitory receptor expressed by exhausted CD8+ T cells. In mouse models of cancer, it has been shown that the most dysfunctional tumor-infiltrating CD8+ T cells actually co-express PD-1 and TIM-3. LAG-3 is another recently identified inhibitory receptor that acts to limit effector T-cell function and augment the suppressive activity of T regulatory cells. It has recently been revealed that PD-1 and LAG-3 are extensively co-expressed by tumor-infiltrating T cells in mice, and that combined blockade of PD-1 and LAG-3 provokes potent synergistic antitumor immune responses in mouse models of cancer.

One embodiment includes the use of immune checkpoint inhibitors in combination with an anti-MMP9 antibody or antigen binding fragment thereof to treat or prevent an MMP9-associated disease or condition. In some embodiments, the immune checkpoint inhibitors may be an anti-PD-1 and/or an anti-PD-L1 antibody. In some embodiments, the anti-PD-L1 antibody may be B7-H1 antibody, BMS 936559 antibody, MPDL3280A (atezolizumab) antibody, MEDI-4736 antibody, MSB0010718C antibody or combinations thereof. According to another embodiment, the anti-PD-1 antibody may be nivolumab antibody, pembrolizumab antibody, pidilizumab antibody or combinations thereof.

In addition, PD-1 may also be targeted with AMP-224, which is a PD-L2-IgG recombinant fusion protein. Additional antagonists of inhibitory pathways in the immune response include IMP321, a soluble LAG-3 Ig fusion protein and MEW class II agonist, which is used to increase an immune response to tumors. Lirilumab is an antagonist to the KIR receptor and BMS 986016 is an antagonist of LAG3. The TIM-3-Galectin-9 pathway is another inhibitory checkpoint pathway that is also a promising target for checkpoint inhibition. RX518 targets and activates the glucocorticoid-induced tumor necrosis factor receptor (GITR), a member of the TNF receptor superfamily that is expressed on the surface of multiple types of immune cells, including regulatory T cells, effector T cells, B cells, natural killer (NK) cells, and activated dendritic cells.

Anti-PD-1 antibodies that may be used in the compositions and methods described herein include but are not limited to: Nivolumab (Opdivo®/MDX-1106/BMS-936558/ONO-4538), a fully human lgG4 anti-PD-1 monoclonal antibody; pidilizumab (MDV9300/CT-011), a humanized IgG1 monoclonal antibody; pembrolizumab (MK-3475/Keytruda®/lambrolizumab), a humanized monoclonal IgG4 antibody; durvalumab (MEDI-4736) and atezolizumab. Anti-PD-L1 antibodies that may be used in compositions and methods described herein include but are not limited to: avelumab; BMS-936559, a fully human IgG4 antibody; atezolizumab (MPDL3280A/RG-7446), a human monoclonal antibody; MEDI4736; MSB0010718C, and MDX1105-01. In certain embodiments, the anti-PD-1 antibody is nivolumab, pembrolizumab, or pidilizumab. In certain embodiments, the anti-PD-L1 antibody is BMS-936559, atezolizumab, or avelumab. In one embodiment, the immune modulating agent inhibits an immune checkpoint pathway. In another embodiment, the immune checkpoint pathway is selected from the group consisting of CTLA-4, LAG-3, B7-H3, B7-H4, Tim3, BTLA, KIR, A2aR, CD200 and PD-1. Additional antibodies that may be used in compositions and methods described herein include the anti-PD-1 and anti-PD-L1 antibodies disclosed in U.S. Pat. Nos. 8,008,449 and 7,943,743, respectively, each of which is herein incorporated by reference in its entirety.

In certain embodiments, one or more additional therapeutic agent is an anti-inflammatory agent. In certain other embodiments, the anti-inflammatory agent is a tumor necrosis factor alpha (TNFα) inhibitor. As used herein, the terms “TNF alpha,” “TNFα,” or “TNF-α” are interchangeable. TNFα is a pro-inflammatory cytokine secreted primarily by macrophages but also by a variety of other cell types including lymphoid cells, mast cells, endothelial cells, cardiac myocytes, adipose tissue, fibroblasts, and neuronal tissue. TNFα is also known as endotoxin-induced factor in serum, cachectin, and differentiation inducing factor. The tumor necrosis factor (TNF) family includes TNF alpha (TNFα), TNF beta (TNFβ), CD40 ligand (CD40L), Fas ligand (FasL), TNF-related apoptosis inducing ligand (TRAIL), and LIGHT (homologous to lymphotoxins, exhibits inducible expression, and competes with HSV glycoprotein D for HVEM, a receptor expressed by T lymphocytes), some of the most important cytokines involved in, among other physiological processes, systematic inflammation, tumor lysis, apoptosis and initiation of the acute phase reaction.

TNFα is initially synthesized and expressed as a 26 kDa transmembrane protein (mTNFα), the extracellular portion of which is subsequently cleaved by TNFα converting enzyme (TACE), to release the soluble 17 kDa protein (sTNFα). TNFα is found to be present in its membrane-bound and secreted form. TNFα has a tendency to form a trimer. An increase in TNFα synthesis or release occurs in disorders such as inflammation.

TNFα binds to tumor necrosis factor receptors (TNF-R). There are two types of TNF receptors that can either be membrane-bound or soluble: TNF-R1 (TNF receptor type 1; CD120a; p55/60) which is expressed in most tissues and TNF-R2 (TNF receptor type 2; CD120b; p′75/80) which is found in cells of the immune system. Though both TNFR1 and TNFR2 interact with both mTNFα and sTNFa, TNFR1 signaling is strongly activated by both mTNFα and sTNFα, while TNFR2 signaling can only be efficiently activated by mTNFα. Each TNF receptor forms homodimers, but they do not heterodimerize with each other. TNF-R1 also contains a death domain that allows it to interact with other death-domain containing adaptor proteins, whereas TNF-R2 lacks a death domain.

TNFα is a potent chemoattractant for neutrophils, and promotes the expression of adhesion molecules on endothelial cells, helping neutrophils migrate. On macrophages TNFα stimulates phagocytosis, and production of interleukin-1 (IL-1) oxidants and the inflammatory lipid prostaglandin E₂.

Rheumatoid arthritis (RA) is a chronic, systemic, articular autoimmune disease of unknown etiology. Patients with RA have inflamed joints in which TNFα is produced in the lining and deeper layers of the synovium by cells of the monocyte/macrophage lineage. It is postulated that the production of TNFα by cells at the cartilage-pannus junction could lead to cartilage degradation in RA. The inflamed joint in rheumatoid arthritis is known to have increased concentrations of the pro-inflammatory cytokines TNFα and interleukin-1 (IL-1) in the synovial fluid.

The most common rheumatoid arthritis therapy involves the use of nonsteroidal anti-inflammatory drugs (NSAIDs) to alleviate symptoms. However, despite the widespread use of NSAIDs, many individuals cannot tolerate the doses necessary to treat the disorder over a prolonged period of time. In addition, NSAIDs merely treat the symptoms of disorder and not the cause. When patients fail to respond to NSAIDs, other DMARDs (Disease Modifying Anti-Rheumatic Drugs) such as methotrexate, gold salts, D-penicillamine, cyclophosphamide and prednisone are used. These drugs have significant toxicities and their mechanism of action remains unknown.

TNFα causes tumor cell necrosis (a process that involves cell swelling, organelle destruction and finally cell lysis) and apoptosis (a process that involves cell shrinking, the formation of condensed bodies and DNA fragmentation). Additionally, TNFα plays a role in the regulation of embryo development and the sleep-wake cycle, lymph node follicle and germinal center formation and host defense against pathogen infection. Importantly, TNFα is a crucial mediator of both acute and chronic systematic inflammatory reactions. TNFα induces its own secretion and stimulates the production of other inflammatory cytokines and chemokines. Animal models of septic shock implicate TNFα as a key player in this condition. TNFα is also a principal player in autoimmune diseases such as rheumatoid arthritis (RA); inflammatory bowel diseases such as Crohn's disease and ulcerative colitis; multiple sclerosis, systemic lupus erythematosus; and systemic sclerosis. Finally, TNFα is associated with tumorigenesis, tumor progression, invasion and metastasis, and is involved in cancer-associated inflammation.

In certain embodiments, TNFα inhibitors are antibodies, peptibodies, avimers, peptide-mimetic compounds, small molecules, or proteins. TNFα inhibitors include, but are not limited to, broad spectrum immunosuppressants (e.g., steroids, including synthetic glucocorticoids such as dexamethasone), curcumin, antibodies, and fusion constructs. Antibodies such as Infliximab (REMICADE®), Adalimumab (HUMIRA®), and receptor-construct fusion proteins such as Etanercept (ENBREL®, Amgen; described in WO 91/03553 and WO 09/406476, herein incorporated by reference in its entirety) are examples of TNFα inhibitors. TNFα inhibitors also include antibodies and other agents which bind to the TNF receptor, thereby inhibiting biological effects of TNFα. In one embodiment, the TNFα inhibitor is a recombinant TNF binding protein (r-TBP-I) (Serono).

In another embodiment, the TNFα inhibitor is a small molecule. In yet another embodiment, the small molecule is selected from the group consisting of pomalidomide, thalidomide, lenalidomide and bupropion. In certain embodiments, the TNFα inhibitor is an antibody. In another embodiment, the antibody is selected from the group consisting of certolizumab pegol, adalimumab, golimumab and infliximab. In another embodiment, the TNFα inhibitor is Etanercept.

Evidence that TNFα is central in the pathogenesis of RA comes from clinical experience with either monoclonal antibodies against TNFα or soluble TNF receptor-immunoglobulin constructs. Five anti-TNFα biologics that block the interaction of TNFα with TNF receptors have received FDA approval for treating rheumatoid arthritis, among other indications. Etanercept (marketed as Enbrel®) is a recombinant fusion protein comprising two p75 soluble TNF-receptor domains linked to the Fc portion of a human immunoglobulin IgG1 and is produced by recombinant DNA technology in a Chinese hamster ovary mammalian cell expression system. Adalilumab (marketed as Humira®) is a recombinant human IgG1 monoclonal antibody expressed in Chinese Hamster Ovary cells. Infliximab (marketed as Remicade®) is a chimeric antibody having murine anti-TNFα variable domains and human IgG1 Fc regions. Certolizumab pegol (marketed as Cimzia®) is a humanized antigen-binding fragment (Fab′) of a monoclonal antibody that has been conjugated to polyethylene glycol. Golimumab (marketed as Simponi®) is a recombinant human IgG1 monoclonal antibody that binds to both soluble and transmembrane forms of TNFα.

Examples of TNF antagonists include SAR-244181, denosumab, etanercept, brentuximab vedotin, AVX-470, BIIB-023, fulranumab, tanezumab, GBR-830, AG-014, lucatumumab, fasinumab, BI-655064, BN-006, ASKP-1240, RNS-60, APG-101, PF-688, APX-005M, ONL-1204, AFM-13, FFP-104, RPH-203, MEDI-578, mDTA-1, AVX-1555, TDI-00846, IDD-004, APX-008, NM-9405, FFP-102, DS-8273, KGYY-15, ONL-101, SCB-808, SCB-131, Atu-614, DE-098, FFP-106, p75NTR-Fc, ANA-02, MEDI-4920, Novotarg, BMS-986090, VAY-736, CD40DNA Vax, GSK-2800528, pegsunercept, GBL-5b, NM-2014, Neutrolide, K-252a, ATROSAB, ABT-110, SAR-127963, 5C-11, ACE-772, ISIS-22023, CRB-0089, oxelumab, enavatuzumab, ALD-906, VT-362, F45D9, F61F12, ALD-901, AMPT1RA, APG-103, E-3330, dacetuzumab, rolipram, AG-879, onercept, D-609, DE-096, EC-234, MDX-1401, BIM-036, ALS-00T2-0501, CZEN-001, P-60 PLAD, PD-90780, LT-ZMP001, CS-9507, PCM-4, toralizumab, DOM-0100, ReN-1820, solimastat, iratumumab, CGEN-40, PN-0615, lenercept, AUX-202, DOM-0800, ITF-1779, CEP-751, daxalipram, B-975, teneliximab, ALE-0540, MDL-201112, and BB-2275.

Anti-TNFα antibodies that may be used include but are not limited to: those described in U.S. Pat. Nos. 6,090,382; 6,258,562; 6,509,015, and in U.S. patent application Ser. Nos. 09/801,185 and 10/302,356, each of which is herein incorporated by reference in its entirety; infliximab (Remicade®, Johnson and Johnson; described in U.S. Pat. No. 5,656,272, herein incorporated by reference in its entirety); CDP571 (a humanized monoclonal anti-TNFα IgG4 antibody); CDP 870 (a humanized monoclonal anti-TNFα antibody fragment); an anti-TNF dAb (Peptech), golimumab (CNTO 148; Medarex and Centocor, see WO 02/12502, herein incorporated by reference in its entirety), and adalimumab (Humira®, Abbott Laboratories, a human anti-TNF mAb, described in U.S. Pat. No. 6,090,382 as D2E7, herein incorporated by reference in its entirety). Additional TNF antibodies which can be used are described in U.S. Pat. Nos. 6,593,458; 6,498,237; 6,451,983; and 6,448,380, each of which is herein incorporated by reference in its entirety.

In certain embodiments, one or more additional therapeutic agent is a chemotherapeutic agent. Chemotherapeutic agents may be categorized by their mechanism of action into, for example, the following groups: anti-metabolites/anti-cancer agents such as pyrimidine analogs floxuridine, capecitabine, and cytarabine; purine analogs, folate antagonists (such as pralatrexate), and related inhibitors; antiproliferative/antimitotic agents including natural products such as vinca alkaloid (vinblastine, vincristine) and microtubule such as taxane (paclitaxel, docetaxel), vinblastin, nocodazole, epothilones, vinorelbine (NAVELBINE®), and epipodophyllotoxins (etoposide, teniposide); DNA damaging agents such as actinomycin, amsacrine, busulfan, carboplatin, chlorambucil, cisplatin, cyclophosphamide (CYTOXAN®), dactinomycin, daunorubicin, doxorubicin, epirubicin, iphosphamide, melphalan, merchlorethamine, mitomycin, mitoxantrone, nitrosourea, procarbazine, taxol, taxotere, teniposide, etoposide, and triethylenethiophosphoramide; antibiotics such as dactinomycin, daunorubicin, doxorubicin, idarubicin, anthracyclines, mitoxantrone, bleomycins, plicamycin (mithramycin), and mitomycin; enzymes such as L-asparaginase which systemically metabolizes L-asparagine and deprives cells which do not have the capacity to synthesize their own asparagine; antiplatelet agents; asparaginase stimulators, such as crisantaspase (Erwinase®) and GRASPA (ERY-001, ERY-ASP); antiproliferative/antimitotic alkylating agents such as nitrogen mustards cyclophosphamide and analogs (melphalan, chlorambucil, hexamethylmelamine, and thiotepa), alkyl nitrosoureas (carmustine) and analogs, streptozocin, and triazenes (dacarbazine); antiproliferative/antimitotic antimetabolites such as folic acid analogs (methotrexate); platinum coordination complexes (cisplatin, oxiloplatinim, lobaplatin, and carboplatin), procarbazine, hydroxyurea, mitotane, and aminoglutethimide; hormones, hormone analogs (estrogen, tamoxifen, goserelin, bicalutamide, and nilutamide), and aromatase inhibitors (letrozole and anastrozole); anticoagulants such as heparin, synthetic heparin salts, and other inhibitors of thrombin; fibrinolytic agents such as tissue plasminogen activator, streptokinase, urokinase, aspirin, dipyridamole, ticlopidine, and clopidogrel; antimigratory agents; antisecretory agents (breveldin); immunosuppressives tacrolimus, sirolimus, azathioprine, and mycophenolate; compounds (TNP-470, genistein) and growth factor inhibitors (vascular endothelial growth factor inhibitors and fibroblast growth factor inhibitors); angiotensin receptor blockers, nitric oxide donors; anti-sense oligonucleotides; antibodies such as trastuzumab and rituximab; cell cycle inhibitors and differentiation inducers such as tretinoin; inhibitors, topoisomerase inhibitors (doxorubicin, daunorubicin, dactinomycin, eniposide, epirubicin, etoposide, idarubicin, irinotecan, mitoxantrone, topotecan, sobuzoxane, and irinotecan), and corticosteroids (cortisone, dexamethasone, hydrocortisone, methylprednisolone, prednisone, and prednisolone); growth factor signal transduction kinase inhibitors; dysfunction inducers; toxins such as Cholera toxin, ricin, Pseudomonas exotoxin, Bordetella pertussis adenylate cyclase toxin, diphtheria toxin, and caspase activators; chromatin; smoothened (SMO) receptor inhibitors, such as Odomzo® (sonidegib, formerly LDE-225), LEQ506, vismodegib (GDC-0449), BMS-833923, glasdegib (PF-04449913), LY2940680, and itraconazole; interferon alpha ligand modulators, such as interferon alfa-2b, interferon alpha-2a biosimilar (Biogenomics), ropeginterferon alfa-2b (AOP-2014, P-1101, PEG IFN alpha-2b), Multiferon (Alfanative, Viragen), interferon alpha 1b, Roferon-A (Canferon, Ro-25-3036), interferon alfa-2a follow-on biologic (Biosidus)(Inmutag, Inter 2A), interferon alfa-2b follow-on biologic (Biosidus—Bioferon, Citopheron, Ganapar)(Beijing Kawin Technology—Kaferon)(AXXO—interferon alfa-2b), Alfaferone, pegylated interferon alpha-1b, peginterferon alfa-2b follow-on biologic (Amega), recombinant human interferon alpha-1b, recombinant human interferon alpha-2a, recombinant human interferon alpha-2b, veltuzumab-IFN alpha 2b conjugate, Dynavax (SD-101), and interferon alfa-n1 (Humoferon, SM-10500, Sumiferon); interferon gamma ligand modulators, such as interferon gamma (OH-6000, Ogamma 100); Complement C3 modulators, such as Imprime PGG; IL-6 receptor modulators, such as tocilizumab, siltuximab, AS-101 (CB-06-02, IVX-Q-101); Telomerase modulators, such as tertomotide (GV-1001, HR-2802, Riavax) and imetelstat (GRN-163, JNJ-63935937); DNA methyltransferases inhibitors, such as temozolomide (CCRG-81045), decitabine, guadecitabine (S-110, SGI-110), KRX-0402, and azacitidine; DNA gyrase inhibitors, such as pixantrone and sobuzoxane; and Bcl-2 family protein inhibitor ABT-263, venetoclax (ABT-199), ABT-737, and AT-101.

Further examples of chemotherapeutic agents include: alkylating agents such as thiotepa and cyclophosphamide (CYTOXAN®); alkyl sulfonates such as busulfan, improsulfan, and piposulfan; aziridines such as benzodepa, carboquone, meturedepa, and uredepa; ethylenimines and methylamelamines including altretamine, triethylenemelamine, triethylenephosphoramide, triethylenethiophosphoramide, and trimemylolomelamine; acetogenins, especially bullatacin and bullatacinone; a camptothecin, including synthetic analog topotecan; bryostatin; callystatin; CC-1065, including its adozelesin, carzelesin, and bizelesin synthetic analogs; cryptophycins, e.g., cryptophycin 1 and cryptophycin 8; dolastatin; duocarmycin, including the synthetic analogs KW-2189 and CBI-TMI; eleutherobin; pancratistatin; a sarcodictyin; spongistatin; nitrogen mustards such as chlorambucil, chlornaphazine, cyclophosphamide, estramustine, ifosfamide, mechlorethamine, mechlorethamine oxide hydrochloride, melphalan, novembichin, phenesterine, prednimustine, trofosfamide, and uracil mustard; nitrosoureas such as carmustine, chlorozotocin, foremustine, lomustine, nimustine, and ranimustine; antibiotics such as the enediyne antibiotics (e.g., calicheamicin, especially calicheamicin gammaII and calicheamicin phiI1), dynemicin including dynemicin A, bisphosphonates such as clodronate, an esperamicin, neocarzinostatin chromophore and related chromoprotein enediyne antibiotic chromomophores, aclacinomycins, actinomycin, authramycin, azaserine, bleomycins, cactinomycin, carabicin, carrninomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin, and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, and zorubicin; anti-metabolites such as methotrexate and 5-fluorouracil (5-FU); folic acid analogs such as demopterin, methotrexate, pteropterin, and trimetrexate; purine analogs such as fludarabine, 6-mercaptopurine, thiamiprine, and thioguanine; pyrimidine analogs such as ancitabine, azacitidine, 6-azauridine, carmofur, cytarabine, dideoxyuridine, doxifluridine, enocitabine, and floxuridine; androgens such as calusterone, dromostanolone propionate, epitiostanol, mepitiostane, and testolactone; anti-adrenals such as aminoglutethimide, mitotane, and trilostane; folic acid replinishers such as frolinic acid; trichothecenes, especially T-2 toxin, verracurin A, roridin A, and anguidine; taxoids such as paclitaxel (TAXOL®) and docetaxel (TAXOTERE®); platinum analogs such as cisplatin and carboplatin; aceglatone; aldophosphamide glycoside; aminolevulinic acid; eniluracil; amsacrine; hestrabucil; bisantrene; edatraxate; defofamine; demecolcine; diaziquone; elformthine; elliptinium acetate; an epothilone; etoglucid; gallium nitrate; hydroxyurea; lentinan; leucovorin; lonidamine; maytansinoids such as maytansine and ansamitocins; mitoguazone; mitoxantrone; mopidamol; nitracrine; pentostatin; phenamet; pirarubicin; losoxantrone; fluoropyrimidine; folinic acid; podophyllinic acid; 2-ethylhydrazide; procarbazine; polysaccharide-K (PSK); razoxane; rhizoxin; sizofiran; spirogermanium; tenuazonic acid; triaziquone; 2,2′,2″-tricUorotriemylamine; urethane; vindesine; dacarbazine; mannomustine; mitobronitol; mitolactol; pipobroman; gacytosine; arabinoside (“Ara-C”); cyclophosphamide; thiopeta; chlorambucil; gemcitabine (GEMZAR®); 6-thioguanine; mercaptopurine; methotrexate; vinblastine; platinum; etoposide (VP-16); ifosfamide; mitroxantrone; vancristine; vinorelbine (NAVELBINE®); novantrone; teniposide; edatrexate; daunomycin; aminopterin; xeoloda; ibandronate; CPT-11; topoisomerase inhibitor RFS 2000; difluoromethylornithine (DFMO); retinoids such as retinoic acid; capecitabine; FOLFIRI (fluorouracil, leucovorin, and irinotecan); and pharmaceutically acceptable salts, acids, or derivatives of any of the above.

Also included in the definition of “chemotherapeutic agent” are anti-hormonal agents such as anti-estrogens and selective estrogen receptor modulators (SERMs), inhibitors of the enzyme aromatase, anti-androgens, and pharmaceutically acceptable salts, acids or derivatives of any of the above that act to regulate or inhibit hormone action on tumors. Examples of anti-estrogens and SERMs include, for example, tamoxifen (including NOLVADEX™), raloxifene, droloxifene, 4-hydroxytamoxifen, trioxifene, keoxifene, LY117018, onapristone, and toremifene (FARESTON®). Inhibitors of the enzyme aromatase regulate estrogen production in the adrenal glands. Examples include 4(5)-imidazoles, aminoglutethimide, megestrol acetate (MEGACE®), exemestane, formestane, fadrozole, vorozole (RIVISOR®), letrozole (FEMARA®), and anastrozole (ARIMIDEX®). Examples of anti-androgens include flutamide, nilutamide, bicalutamide, leuprohde, and goserelin.

Anti-angiogenic agents include, but are not limited to, retinoid acid and derivatives thereof, 2-methoxyestradiol, ANGIOSTATIN®, ENIDOSTATIN®, suramin, squalamine, tissue inhibitor of metalloproteinase-1, tissue inhibitor of metalloproteinase-2, plasminogen activator inhibitor-1, plasminogen activator inhibitor-2, cartilage-derived inhibitor, paclitaxel (nab-paclitaxel), platelet factor 4, protamine sulphate (clupeine), sulphated chitin derivatives (prepared from queen crab shells), sulphated polysaccharide peptidoglycan complex (sp-pg), staurosporine, modulators of matrix metabolism including proline analogs such as 1-azetidine-2-carboxylic acid (LACA), cishydroxyproline, d,I-3,4-dehydroproline, thiaproline, α,α′-dipyridyl, beta-aminopropionitrile fumarate, 4-propyl-5-(4-pyridinyl)-2(3h)-oxazolone, methotrexate, mitoxantrone, heparin, interferons, interferon alpha ligand modulators, 2 macroglobulin-serum, chicken inhibitor of metalloproteinase-3 (ChIMP-3), chymostatin, beta-cyclodextrin tetradecasulfate, eponemycin, fumagillin, gold sodium thiomalate, d-penicillamine, beta-1-anticollagenase-serum, alpha-2-antiplasmin, bisantrene, lobenzarit disodium, n-2-carboxyphenyl-4-chloroanthronilic acid disodium or “CCA”, thalidomide, angiostatic steroid, carboxy aminoimidazole, and metalloproteinase inhibitors such as BB-94. Other anti-angiogenesis agents include antibodies, preferably monoclonal antibodies against these angiogenic growth factors: beta-FGF, alpha-FGF, FGF-5, VEGF isoforms, VEGF-C, HGF/SF, and Ang-1/Ang-2.

Anti-fibrotic agents include, but are not limited to, the compounds such as beta-aminoproprionitrile (BAPN), as well as the compounds disclosed in U.S. Pat. No. 4,965,288 relating to inhibitors of lysyl oxidase and their use in the treatment of diseases and conditions associated with the abnormal deposition of collagen and U.S. Pat. No. 4,997,854 relating to compounds which inhibit LOX for the treatment of various pathological fibrotic states, which are herein incorporated by reference. Further exemplary inhibitors are described in U.S. Pat. No. 4,943,593 relating to compounds such as 2-isobutyl-3-fluoro-, chloro-, or bromo-allylamine, U.S. Pat. No. 5,021,456, U.S. Pat. No. 5,059,714, U.S. Pat. No. 5,120,764, U.S. Pat. No. 5,182,297, U.S. Pat. No. 5,252,608 relating to 2-(1-naphthyloxymemyl)-3-fluoroallylamine, and US 2004-0248871, which are herein incorporated by reference.

Exemplary anti-fibrotic agents also include the primary amines reacting with the carbonyl group of the active site of the lysyl oxidases, and more particularly those which produce, after binding with the carbonyl, a product stabilized by resonance, such as the following primary amines: emylenemamine, hydrazine, phenylhydrazine, and their derivatives; semicarbazide and urea derivatives; aminonitriles such as BAPN or 2-nitroethylamine; unsaturated or saturated haloamines such as 2-bromo-ethylamine, 2-chloroethylamine, 2-trifluoroethylamine, 3-bromopropylamine, and p-halobenzylamines; and selenohomocysteine lactone. Other anti-fibrotic agents are copper chelating agents penetrating or not penetrating the cells. Exemplary compounds include indirect inhibitors which block the aldehyde derivatives originating from the oxidative deamination of the lysyl and hydroxylysyl residues by the lysyl oxidases. Examples include the thiolamines, e.g., D-penicillamine, and its analogs such as 2-amino-5-mercapto-5-methylhexanoic acid, D-2-amino-3-methyl-3-((2-acetamidoethyl)dithio)butanoic acid, p-2-amino-3-methyl-3-((2-aminoethyl)dithio)butanoic acid, sodium-4-((p-1-dimethyl-2-amino-2-carboxyethyl)dithio)butane sulphurate, 2-acetamidoethyl-2-acetamidoethanethiol sulphanate, and sodium-4-mercaptobutanesulphinate trihydrate.

Immunotherapeutic agents include and are not limited to therapeutic antibodies suitable for treating patients. Some examples of therapeutic antibodies include simtuzumab, abagovomab, adecatumumab, afutuzumab, alemtuzumab, altumomab, amatuximab, anatumomab, arcitumomab, bavituximab, bectumomab, bevacizumab, bivatuzumab, blinatumomab, brentuximab, cantuzumab, catumaxomab, cetuximab, citatuzumab, cixutumumab, clivatuzumab, conatumumab, daratumumab, drozitumab, duligotumab, dusigitumab, detumomab, dacetuzumab, dalotuzumab, ecromeximab, elotuzumab, ensituximab, ertumaxomab, etaracizumab, farletuzumab, ficlatuzumab, figitumumab, flanvotumab, futuximab, ganitumab, gemtuzumab, girentuximab, glembatumumab, ibritumomab, igovomab, imgatuzumab, indatuximab, inotuzumab, intetumumab, ipilimumab (YERVOY®, MDX-010, BMS-734016, and MDX-101), iratumumab, labetuzumab, lexatumumab, lintuzumab, lorvotuzumab, lucatumumab, mapatumumab, matuzumab, milatuzumab, minretumomab, mitumomab, moxetumomab, narnatumab, naptumomab, necitumumab, nimotuzumab, nofetumomab, obinutuzumab, ocaratuzumab, ofatumumab, olaratumab, onartuzumab, oportuzumab, oregovomab, panitumumab, parsatuzumab, patritumab, pemtumomab, pertuzumab, pintumomab, pritumumab, racotumomab, radretumab, rilotumumab, rituximab, robatumumab, satumomab, sibrotuzumab, siltuximab, solitomab, tacatuzumab, taplitumomab, tenatumomab, teprotumumab, tigatuzumab, tositumomab, trastuzumab, tucotuzumab, ublituximab, veltuzumab, vorsetuzumab, votumumab, zalutumumab, CC49, and 3F8. Rituximab can be used for treating indolent B-cell cancers, including marginal-zone lymphoma, WM, CLL and small lymphocytic lymphoma. A combination of Rituximab and chemotherapy agents is especially effective. The exemplified therapeutic antibodies may be further labeled or combined with a radioisotope particle such as indium-111, yttrium-90, or iodine-131.

In certain embodiments, the one or more additional therapeutic agent includes and is not limited an A2B inhibitor, an apoptosis signal-regulating kinase (ASK) inhibitor, a Bruton's tyrosine kinase (BTK) inhibitor, a BET-bromodomain 4 (BRD4) inhibitor, a casein kinase inhibitor, a cyclin dependent kinase (CDK) inhibitor, a discoidin domain receptor (DDR) inhibitor, a histone deacetylase (HDAC) inhibitor, a protein kinase HPK1 inhibitor, an isocitrate dehydrogenase (IDH) inhibitor, an IDO1 inhibitor, a Janus kinase (JAK) inhibitor, a lysyl oxidase-like protein (LOXL) inhibitor, a MEK inhibitor, a matrix metalloprotease (MMP) inhibitor, an IKK inhibitor, phosphatidylinositol 3-kinase (PI3K) inhibitor, a protein kinase C (PKC) activator or inhibitor, agents that activate or reactivate latent human immunodeficiency virus (HIV) such as panobinostat or romidepsin, an anti-CD20 antibody such as obinutuzumab, an anti-programmed cell death protein 1 (PD-1) inhibitor such as nivolumab (OPDIVO®, BMS-936558, MDX1106, or MK-34775), durvalumab (MEDI-4736), atezolizumab, and pembrolizumab (KEYTRODA®, MK-3475, SCH-900475, lambrolizumab), an anti-programmed death-ligand 1 (anti-PD-L1) inhibitor such as BMS-936559, MPDL3280A, MEDI4736, MSB0010718C, and MDX1105-01, a spleen tyrosine kinase (SYK) inhibitor, a serine/threonine-protein kinase 1 (TBK1) inhibitor, a TPL2 inhibitor, and a smoothened (SMO) receptor inhibitor. These agents may be in the forms of compound, antibodies, polypeptide, or polynucleotides. In the present application, the MMP9 binding protein, including anti-MMP9 antibody such as AB0045, may be used or combined with the above one or more therapeutic agent, and may be further used or combined with a chemotherapeutic agent, an anti-angiogenic agent, an anti-fibrotic agent, an anti-inflammatory agent, an immune modulating agent, an immunotherapeutic agent, a therapeutic antibody, a radiotherapeutic agent, an anti-neoplastic agent or an anti-cancer agent, an anti-proliferation agent, or any combination thereof. It is understood that some agents may be considered or used for more than one disease type; for example, an agent may be considered or used for anti-inflammation or anti-cancer, accordingly, may be used or combined with anti-MMP9 antibody of the present application for treating or preventing inflammation, auto-immune, or cancers.

Additional examples of one or more additional therapeutic agents may include and is not limited to hedgehog protein inhibitors, smoothened receptor antagonists, endothelin ET-A antagonists, endothelin ET-B antagonists, FGF receptor antagonists, FGF1 receptor antagonists, FGF2 receptor antagonists, PDGF receptor alpha antagonists, PDGF receptor antagonists, PDGF receptor beta antagonists, VEGF receptor antagonists, VEGF-1 receptor antagonists, VEGF-2 receptor antagonists, VEGF-3 receptor antagonists, IL-13 antagonists, interferon beta ligands, mTOR complex 1 inhibitors, TGF beta antagonists, p38 MAP kinase inhibitors, NADPH oxidase 1 inhibitors, NADPH oxidase 4 inhibitors, connective tissue growth factor ligand inhibitors, IL-6 antagonists, IL-6 agonists, insulin-like growth factor 1 antagonists, somatostatin receptor agonists, 5-lipoxygenase inhibitors, PDE 3 inhibitors, phospholipase C inhibitors, serum amyloid P stimulator, guanylate cyclase stimulator, PDE 4 inhibitors, Abl tyrosine kinase inhibitors, Kit tyrosine kinase inhibitors, signal transduction inhibitors, angiotensin II ligand modulator, endothelin 1 ligand inhibitors, relaxin agonist, IL-4 antagonist, TNF antagonist, type II TNF receptor modulator, monocyte chemotactic protein 1 ligand inhibitors, galectin-3 inhibitors, SH2 domain inositol phosphatase 1 stimulator, MAPKAPK2 inhibitors, caspase inhibitors, lysophosphatidate-1 receptor antagonist, beta 2 adrenoceptor agonist, interferon gamma ligands, superoxide dismutase modulator, hyaluronidase stimulator, transaminase stimulator, integrin alpha-V/beta-6 antagonist, a lysyl oxidase-like protein 2 (LOXL2) inhibitor, adrenoceptor antagonist, VIP agonist, interferon alpha ligands, Jun N terminal kinase inhibitors, collagen V modulators, MMP9 stimulators, PPAR agonists, adenosine A2b receptor antagonists, GPCR modulators, CCR7 chemokine modulators, interleukin 17E ligand inhibitors, interleukin receptor 17B antagonists, AKT protein kinase inhibitors, hyaluronan mediated motility receptor modulators, angiotensin II AT-2 receptor agonists, CXC11 chemokine ligand modulators, immunoglobulin Fc receptor modulators, lysophosphatidate-1 receptor antagonists, ubiquitin thioesterase inhibitors, 5-HT 2b receptor antagonists, LDL receptor related protein-6 inhibitors, telomerase stimulators, endostatin modulators, Wnt-1 induced signal pathway protein 1 inhibitors, NK1 receptor antagonists, CD95 antagonists, protein tyrosine phosphatase 1E inhibitors, plasminogen activator inhibitors 1 inhibitors, spleen tyrosine kinase inhibitors, MMP2 inhibitors, MMP3 inhibitors, MMP7 inhibitors, MMP8 inhibitors, TPL2 COT Kinase inhibitors, JAK1/2 inhibitors, JAK1/3 inhibitors, JAK2/3 inhibitors, integrin alpha 4 beta 7 inhibitors, PAD4 inhibitors, PAD2 inhibitors, IRAK4 inhibitors, ASK1 inhibitors, PIM1 inhibitors, PIM3 inhibitors, complement pathway inhibitors, AMPK inhibitors, IL-17 inhibitors, PD-1 agonist, IL-33 inhibitor, IL-25 inhibitors, and IL-22 agonists.

In certain embodiments, the one or more additional therapeutic agents may be selected from vismodegib, macitentan, nintedanib, tralokinumab, ambrisentan, bosentan, interferon beta-1a, everolimus, GKT-137831, PBI-4050, PLX stem cell therapy (Pluristem/Cha Bio & Diostech), lanreotide, tipelukast, INT-0024, PRM-151, riociguat, roflumilast, imatinib, serelaxin, SAR-156597, etanercept, AEOL-10150, lebrikizumab, MPC-300-IV, FG-3019, carlumab, GR-MD-02, AQX-1125, MMI-0100, pirfenidone, deuterated pirfenidone analogs (e.g. SD-560), emricasan, Conatus, BMS-986020, beclometasone dipropionate+formoterol fumarate, TD-139, recombinant midismase, QAX-576, bovhyaluronidase azoximer, GNI/AFTF-351, BG-00011, simtuzumab, SPL-334, pentoxifylline+N-acetyl-cysteine, aviptadil, interferon-alpha, GSK-2126458, actimmune, bentamapimod, CKD-942, tanzisertib, interferon gamma, IW-001, PUR-1500, DB-029.01, disitertide, fresolimumab, IVA-337, PBF-1250, P-013, P-007, anti-IL-17BR humanized antibody, triciribine, RHAMM modulators, RES-529, MOR-107, hR-411, HEC-00000585, BOT-191, GKT-901, USP-34 inhibitors, anti-LRP6 mAb, Gestelmir, Neumomir, IBIO-CFB-03, MSM-735, LTI-03, anti-WISP1 antibodies, NAS-911B, C-301, STNM-04, TM-5441, PP-0612, QU-100, HR-017, Gal-100, MAI-100, BPS-03251, MMP9 antibodies, such as those disclosed in U.S. Pat. No. 8,377,443, ASK-1 inhibitors, such as those disclosed in U.S. Pat. No. 8,378,108, SYK inhibitors, such as those disclosed in US2015/0175616 and U.S. Pat. No. 8,450,321, for example, 6-(1H-indazol-6-yl)-N-(4-morpholinophenyl)imidazo[1,2-a]pyrazin-8-amine), inhibitors of Bruton's tyrosine kinase such as those disclosed in U.S. Pat. No. 8,557,803, for example, (R)-6-amino-9-(1-(but-2-ynoyl)pyrrolidin-3-yl)-7-(4-phenoxyphenyl)-7H-purin-8(9H)-one, FXR agonists such as those disclosed in US20140221659, and PI3K inhibitors, such as those disclosed in US20140371246.

Further examples of the one or more additional therapeutic agents may comprise a kinase or enzyme modulator of, but not limited to, Abl, activated CDC kinase (ACK) such as ACK1, adenosine A2B receptor (A2B), apoptosis signal-regulating kinase (ASK), Aurora kinase, Bruton's tyrosine kinase (BTK), BET-bromodomain (BRD) such as BRD4, c-Kit, c-Met, CDK-activating kinase (CAK), calmodulin-dependent protein kinase (CaMK), cyclin-dependent kinase (CDK), casein kinase (CK), discoidin domain receptor (DDR), epidermal growth factor receptors (EGFR), focal adhesion kinase (FAK), Flt-3, farnesoid x receptor (FXR), FYN, glycogen synthase kinase (GSK), HCK, histone deacetylase (HDAC), indoleamine 2,3-dioxygenase (IDO), I-Kappa-B kinase (IKK) such as IKKβε, isocitrate dehydrogenase (IDH) such as IDH1, Janus kinase (JAK), KDR, lysine demethylase (KDMS), lymphocyte-specific protein tyrosine kinase (LCK), lysyl oxidase protein (LOX), lysyl oxidase-like protein (LOXL), LYN, matrix metalloprotease (MMP), mitogen-activated protein kinase (MEK), mitogen-activated protein kinase (MAPK), mut T homolog (MTH), NEK9, NPM-ALK, p38 kinase, platelet-derived growth factor (PDGF), phosphorylase kinase (PK), polo-like kinase (PLK), phosphatidylinositol 3-kinase (PI3K), protein kinase (PK) such as protein kinase A, B, and/or C, PYK, spleen tyrosine kinase (SYK), serine/threonine kinase TPL2, serine/threonine kinase (STK), signal transduction and transcription (STAT), SRC, serine/threonine-protein kinase (TBK) such as TBK1, TIE, tyrosine kinase (TK), tank-binding kinase (TBK), vascular endothelial growth factor receptor (VEGFR), YES, or any combination thereof.

Apoptosis Signal-Regulating Kinase (ASK1) inhibitors include, but are not limited to, those described in WO 2011/008709 and WO 2013/112741.

Examples of Bruton's tyrosine kinase (BTK) inhibitors include, but are not limited to, (S)-6-amino-9-(1-(but-2-ynoyl)pyrrolidin-3-yl)-7-(4-phenoxyphenyl)-7H-purin-8(9H)-one, ibrutinib, HM71224, ONO-4059, and CC-292, acalabrutinib (ACP-196), PRN-1008, BGB-3111, TAK-020, M-2951, dasatinib, M-2951, HCL-1401, HM-71224, PRN-1008, TAS-5315, BGB-3111, AS-550, DR-109, TAK-020, SNS-062, ONO-4059, X-022, TP-4207, KBP-7536, GDC-0834, ONO-WG-307, and LFM-A13.

Mitogen-activated protein kinase (MAPK) inhibitors include selumetinib (AZD6244), MT-144, sorafenib, trametinib (GSK1120212), binimetinib, antroquinonol, uprosertib+trametinib,

CK inhibitors include CK1 and/or CK2.

CDK inhibitors include inhibitors of CDK 1, 2, 3, 4, and/or 6. Examples of CDK inhibitors include rigosertib, selinexor, UCN-01, alvocidib (HMR-1275, flavopiridol), FLX-925, AT-7519, abemaciclib, palbociclib, and TG-02.

Discoidin Domain Receptor (DDR) Inhibitors include inhibitors of DDR1 and/or DDR2. Examples of DDR inhibitors include, but are not limited to, those disclosed in WO 2014/047624, US 2009-0142345, US 2011-0287011, WO 2013/027802, and WO 2013/034933.

Histone Deacetylase (HDAC) inhibitors include, but are not limited to, pracinostat, CS-055 (HBI-8000), resminostat, entinostat, abexinostat, belinostat, vorinostat, riclinostat, CUDC-907, ACY-241, CKD-581, SHP-141, valproic acid (VAL-001), givinostat, quisinostat (JNJ-26481585), BEBT-908 and panobinostat.

Janus Kinase (JAK) inhibitors inhibit JAK1, JAK2, and/or JAK3, and/or Tyk 2. Examples of JAK inhibitors include, but are not limited to, momelotinib (CYT0387), ruxolitinib, filgotinib (GLPG0634), peficitinib (ASP015K), fedratinib, tofacitinib (formerly tasocitinib), baricitinib, lestaurtinib, pacritinib (SB1518), XL019, AZD1480, INCB039110, LY2784544, BMS911543, AT9283, and NS018. Examples of Janus Kinase inhibitors (e.g. JAK1 and JAK2) include ABT-494, ganetespib, tofacitinib, PF-04965842, ruxolitinib, pacritinib, CF-102, momelotinib, baricitinib, CS-944X, AT-9283, TG-02, AR-13154, ENMD-2076, VR-588, YJC-50018, INCB-39110, NS-018, GLPG-0555, G5-7, BVB-808, INCB-52793, fedratinib, PF-06263276, TP-0413, INCB-47986, CT-1578, peficitinib, BMS-911543, XL-019, solcitinib, MRK-12, AC-410, NMS-P953, CPL-407-22, CPL-407-105, AZD-1480, gandotinib, INCB-016562, CEP-33779, ON-044580, lestaurtinib, K-454, LS-104, SGI-1252, and EXEL-8232.

Lysyl Oxidase-Like Protein (LOXL) inhibitors include inhibitors of LOXL1, LOXL2, LOXL3, LOXL4, and/or LOXL5. Examples of LOXL inhibitors include, but are not limited to, the antibodies described in WO 2009/017833. Examples of LOXL2 inhibitors include, but are not limited to, the antibodies described in WO 2009/017833, WO 2009/035791, and WO 2011/097513. In certain embodiments, the LOXL2 inhibitor is an anti-LOXL2 antibody (see, e.g., U.S. Pat. No. 8,461,303, and U.S. Publication Nos. 2012/0309020, 2013/0324705, and 2014/0079707, each of which are incorporated herein by reference in their entirety). The anti-LOXL2 antibody can be a monoclonal antibody (including full length monoclonal antibody), polyclonal antibody, human antibody, humanized antibody, chimeric antibody, diabody, multispecific antibody (e.g., bispecific antibody), or an antibody fragment including, but not limited to, a single chain binding polypeptide, so long as it exhibits the desired biological activity. Exemplified anti-LOXL2 antibody or antigen binding fragment thereof may be found in U.S. Publication Nos. 2012/0309020, 2013/0324705, 2014/0079707, 2009/0104201, 2009/0053224, and 2011/0200606, each of which is incorporated herein by reference in the entirety).

Polo-like Kinase (PLK) inhibitors include inhibitors of PLK 1, 2, and 3.

Phosphatidylinositol 3-kinase (PI3K) inhibitors include inhibitors of PI3Kγ, PI3Kδ, PI3Kβ, PI3Kα, and/or pan-PI3K. Examples of PI3K inhibitors include, but are not limited to, wortmannin, BKM120, CH5132799, XL756, idelalisib (Zydelig®), and GDC-0980. Examples of PI3Kγ inhibitors include, but are not limited to, ZSTK474, AS252424, LY294002, and TG100115. Examples of PI3Kδ inhibitors include, but are not limited to, PI3K II, TGR-1202, AMG-319, GSK2269557, X-339, X-414, RP5090, KAR4141, XL499, OXY111A, IPI-145, IPI-443, and the compounds described in WO 2005/113556, WO 2013/052699, WO 2013/116562, WO 2014/100765, WO 2014/100767, and WO 2014/201409. Examples of P13Kβ inhibitors include, but are not limited to, GSK2636771, BAY 10824391, and TGX221. Examples of PI3Kα inhibitors include, but are not limited to, buparlisib, BAY 80-6946, BYL719, PX-866, RG7604, MLN1117, WX-037, AEZA-129, and PA799. Examples of pan-PI3K inhibitors include, but are not limited to, LY294002, BEZ235, XL147 (SAR245408), and GDC-0941.

Spleen Tyrosine Kinase (SYK) inhibitors include, but are not limited to, 6-(1H-indazol-6-yl)-N-(4-morpholinophenyl)imidazo[1,2-a]pyrazin-8-amine, tamatinib (R406), fostamatinib (R788), PRT062607, BAY-61-3606, NVP-QAB 205 AA, R112, R343, and those described in U.S. Pat. No. 8,450,321, and those described in U.S. Publication No. 2015/0175616, which is incorporated by reference herein in its entirety.

Tyrosine-kinase Inhibitors (TKIs) may target epidermal growth factor receptors (EGFRs) and receptors for fibroblast growth factor (FGF), platelet-derived growth factor (PDGF), and vascular endothelial growth factor (VEGF). Examples of TKIs that target EGFR include, but are not limited to, gefitinib, nintedanib, and erlotinib. Sunitinib is a non-limiting example of a TKI that targets receptors for FGF, PDGF, and VEGF. Additional TKIs include dasatinib and ponatinib.

Toll-like Receptor (TLR) modulators include inhibitors of TLR-1, TLR-2, TLR-3, TLR-4, TLR-5, TLR-6, TLR-7, TLR-8, TLR-9, TLR-10, TLR-11, TLR-12, and/or TLR-13.

### Therapeutic Use

In certain embodiments, methods are provided for treating or preventing a disease or condition, including any of those described herein, e.g., cystic fibrosis, cancers, autoimmune or inflammatory diseases or conditions, comprising providing to the subject: an effective amount of an MMP9 binding protein comprising an immunoglobulin heavy chain polypeptide, or functional fragment thereof, and an immunoglobulin light chain polypeptide, or functional fragment thereof, wherein the MMP9 binding protein specifically binds MMP9; and an effective amount of an additional therapeutic agent, thereby treating or preventing the MMP9-associated disease or condition in the subject. In one embodiment, the disease or condition comprises myeloid cell-associated inflammation.

In another embodiment, the disease or condition is a cancer selected from the group consisting of: pancreatic cancer, esophagogastric adenocarcinoma, non-small cell lung cancer, lung squamous cell carcinoma, breast cancer, lung adenocarcinoma, gastric adenocarcinoma, colorectal carcinoma, pancreatic adenocarcinoma, head and neck squamous cell carcinoma, hepatocellular carcinoma, colorectal cancer, colorectal adenocarcinoma and hepatocellular carcinoma. In a further embodiment, the disease or condition is an autoimmune or inflammatory disease or condition.

In another embodiment, the autoimmune or inflammatory disease or condition is rheumatoid arthritis, an inflammatory bowel disease (IBD), vasculitis, septicemia, multiple sclerosis, muscular dystrophy, lupus, allergy, asthma or hidradenitis suppurativa. In yet another embodiment, the inflammatory bowel disease is selected from the group consisting of: ulcerative colitis (UC), Crohn's disease (CD), or indeterminate colitis. In yet another embodiment, the vasculitis is giant cell arteritis.

In certain embodiments, methods are provided for treating or preventing one or more cancers, comprising providing to the subject: an effective amount of an MMP9 binding protein comprising an immunoglobulin heavy chain polypeptide, or functional fragment thereof, and an immunoglobulin light chain polypeptide, or functional fragment thereof, wherein the MMP9 binding protein specifically binds MMP9; and an effective amount of an immune checkpoint inhibitor, thereby treating or preventing the one or more cancers in the subject. In one embodiment, the one or more cancers is selected from the group consisting of: pancreatic cancer, esophagogastric adenocarcinoma, non-small cell lung cancer, lung squamous cell carcinoma, lung adenocarcinoma, gastric adenocarcinoma, colorectal carcinoma, pancreatic adenocarcinoma, head and neck squamous cell carcinoma, hepatocellular carcinoma, colorectal cancer, colorectal adenocarcinoma and hepatocellular carcinoma. In another embodiment, the immune checkpoint inhibitor is selected from the group consisting of an anti-PD-1 antibody and an anti-PD-L1 antibody. In certain embodiments, the anti-PD-1 antibody is nivolumab, pembrolizumab, or pidilizumab. In certain embodiments, the anti-PD-L1 antibody is BMS-936559, atezolizumab, or avelumab. In certain embodiments, the MMP9 binding protein is AB0045 or a functional fragment or variant thereof.

In certain embodiments, methods are provided for treating or preventing cystic fibrosis, autoimmune diseases or conditions, or inflammatory diseases or conditions, comprising providing to the subject: an effective amount of an Matrix Metalloproteinase 9 (MMP9) binding protein comprising an immunoglobulin heavy chain polypeptide, or functional fragment thereof, and an immunoglobulin light chain polypeptide, or functional fragment thereof, wherein the MMP9 binding protein specifically binds MMP9; and an effective amount of a TNFα inhibitor, thereby treating or preventing cystic fibrosis, autoimmune or inflammatory diseases or conditions in the subject. In one embodiment, the autoimmune disease or condition, or inflammatory disease or condition is rheumatoid arthritis, an inflammatory bowel disease (IBD), vasculitis, septicemia, multiple sclerosis, muscular dystrophy, lupus, allergy, asthma or hidradenitis suppurativa. In yet another embodiment, the inflammatory bowel disease is selected from the group consisting of: ulcerative colitis (UC), Crohn's disease (CD), or indeterminate colitis. In yet another embodiment, the vasculitis is giant cell arteritis. In certain embodiments, the TNFα inhibitor is an antibody. In another embodiment, the antibody is selected from the group consisting of certolizumab pegol, adalimumab, golimumab and infliximab. In another embodiment, the TNFα inhibitor is Etanercept. In certain embodiments, the MMP9 binding protein is AB0045 or a functional fragment or variant thereof.

In some embodiments, an MMP9 binding protein is used in treating subjects having gastric adenocarcinoma or gastric cancer. In some embodiments, the subjects are administered the MMP9 binding protein intravenously. In certain embodiments, the MMP9 binding protein is administered at about 800 mg. In other embodiments, the subjects are administered the MMP9 binding protein every two weeks. In some aspects of such embodiments, the patients are administered the MMP9 binding protein intravenously at a dosage of 800 mg every two weeks.

In some embodiments, an MMP9 binding protein is used in treating subjects having cystic fibrosis, non-cystic fibrosis bronchiectasis, sarcoidosis, idiopathic pulmonary fibrosis, tuberculosis, a cancer, autoimmune or inflammatory diseases or conditions. In some embodiments, the subjects are administered the MMP9 binding protein with a Janus kinase (JAK) inhibitor. In some embodiments, the JAK inhibitor is filgotinib.

In certain embodiments of any of the compositions or methods for treating or preventing a disease or condition, the anti-MMP9 antibody or antigen binding fragment thereof is AB0045 and the immune modulating agent is etanercept. In another embodiment, the anti-MMP9 antibody or antigen binding fragment thereof is AB0045 and the immune modulating agent is adalilumab. In another embodiment, the anti-MMP9 antibody or antigen binding fragment thereof is AB0045 and the immune modulating agent is infliximab. In another embodiment, the anti-MMP9 antibody or antigen binding fragment thereof is AB0045 and the immune modulating agent is nivolumab. In another embodiment, the anti-MMP9 antibody or antigen binding fragment thereof is AB0045 and the immune modulating agent is pembrolizumab. In another embodiment, the anti-MMP9 antibody or antigen binding fragment thereof is AB0045 and the immune modulating agent is pidilizumab. In another embodiment, the anti-MMP9 antibody or antigen binding fragment thereof is AB0045 and the immune modulating agent is BMS-936559. In another embodiment, the anti-MMP9 antibody or antigen binding fragment thereof is AB0045 and the immune modulating agent is atezolizumab. In one embodiment, the anti-MMP9 antibody or antigen binding fragment thereof is AB0045 and the immune modulating agent is certolizumab pegol. In one embodiment, the anti-MMP9 antibody or antigen binding fragment thereof is AB0045 and the immune modulating agent is golimumab. In another embodiment, the anti-MMP9 antibody or antigen binding fragment thereof is AB0045 and the immune modulating agent is nivolumab. In another embodiment, the anti-MMP9 antibody or antigen binding fragment thereof is AB0045 and the immune modulating agent is avelumab.

In one embodiment of any of the compositions or methods for treating or preventing a disease or condition, the additional therapeutic agent is a tumor necrosis factor alpha (TNFα) inhibitor selected from the group consisting of Etanercept, pomalidomide, thalidomide, lenalidomide and bupropion, certolizumab pegol, adalimumab, golimumab and infliximab. In one embodiment of any of the compositions or methods for treating or preventing a disease or condition, the additional therapeutic agent is selected from the group consisting of an anti-PD-1 antibody and an anti-PD-L1 antibody. In certain embodiments, the anti-PD-1 antibody is nivolumab, pembrolizumab, or pidilizumab. In certain embodiments, the anti-PD-L1 antibody is BMS-936559, atezolizumab, or avelumab. In one embodiment, the additional therapeutic agent inhibits an immune checkpoint pathway. In another embodiment, the immune checkpoint pathway is selected from the group consisting of CTLA-4, LAG-3, B7-H3, B7-H4, Tim3, BTLA, KIR, A2aR, CD200 and PD-1. In one embodiment of any of the compositions or methods for treating or preventing a disease or condition, the anti-MMP9 antibody or antigen binding fragment thereof is AB0045 and the immune modulating agent is etanercept. In another embodiment, the anti-MMP9 antibody or antigen binding fragment thereof is AB0045 and the immune modulating agent is adalilumab. In another embodiment, the anti-MMP9 antibody or antigen binding fragment thereof is AB0045 and the immune modulating agent is infliximab. In another embodiment, the anti-MMP9 antibody or antigen binding fragment thereof is AB0045 and the immune modulating agent is nivolumab. In another embodiment, the anti-MMP9 antibody or antigen binding fragment thereof is AB0045 and the immune modulating agent is pembrolizumab. In another embodiment, the anti-MMP9 antibody or antigen binding fragment thereof is AB0045 and the immune modulating agent is pidilizumab. In another embodiment, the anti-MMP9 antibody or antigen binding fragment thereof is AB0045 and the immune modulating agent is BMS-936559. In another embodiment, the anti-MMP9 antibody or antigen binding fragment thereof is AB0045 and the immune modulating agent is atezolizumab.

In certain embodiments, the one or more addition therapeutic agent is selected from the group consisting of an antibody, a small molecule and a recombinant molecule. In some embodiments, the additional therapeutic agent is a tumor necrosis factor alpha (TNFα) inhibitor. In another embodiment, the TNFα inhibitor is a small molecule. In yet another embodiment, the small molecule is selected from the group consisting of pomalidomide, thalidomide, lenalidomide and bupropion. In certain embodiments, the TNFα inhibitor is an antibody. In another embodiment, the antibody is selected from the group consisting of certolizumab pegol, adalimumab, golimumab and infliximab. In another embodiment, the TNFα inhibitor is Etanercept.

In some embodiments, the additional therapeutic agent is selected from the group consisting of an anti-PD-1 antibody and an anti-PD-L1 antibody. In certain embodiments, the anti-PD-1 antibody is nivolumab, pembrolizumab, or pidilizumab. In certain embodiments, the anti-PD-L1 antibody is BMS-936559, atezolizumab, or avelumab. In one embodiment, the immune modulating agent inhibits an immune checkpoint pathway. In another embodiment, the immune checkpoint pathway is selected from the group consisting of CTLA-4, LAG-3, B7-H3, B7-H4, Tim3, BTLA, KIR, A2aR, CD200 and PD-1.

In certain embodiments, the anti-MMP9 antibody or antigen binding fragment thereof and the additional therapeutic agents(s), e.g., immune modulating agent, can be administered concurrently or sequentially. Concurrent administration of the anti-MMP9 antibody or antigen binding fragment thereof and the other therapeutic agent or their compositions comprises administration at the same time or at a time that overlaps. Sequential administration of the anti-MMP9 antibody or antigen binding fragment thereof and the immune modulating agent or their compositions comprises administration of either the anti-MMP9 antibody or antigen binding fragment thereof or its compositions first, followed by administration of the immune modulating agent or its composition second, or vice versa.

In some embodiments, the anti-MMP9 antibody or antigen binding fragment thereof of the present disclosure may be used as the primary or front-line agent and the additional agent may be used as the secondary agent. In other embodiments, the additional therapeutic agent may be used as the primary or front-line agent and the anti-MMP9 antibody or antigen binding fragment thereof may be used as the secondary agent.

The one or more additional therapeutic agents can be an agent useful for the treatment of cancer and related conditions. In some embodiments, the present disclosure provides methods for treating or preventing a disease or condition such as cystic fibrosis, cancers, autoimmune diseases or conditions, or inflammatory diseases or conditions, comprising providing to the subject: (i) an effective amount of an Matrix Metalloproteinase 9 (MMP9) binding protein comprising an immunoglobulin heavy chain polypeptide, or functional fragment thereof, and an immunoglobulin light chain polypeptide, or functional fragment thereof, wherein the MMP9 binding protein specifically binds MMP9; and (ii) an effective amount of an immune modulating agent; and (iii) an effective amount of one or more additional therapeutic agents that is an anti-tumor agent or oncology agent, thereby treating or preventing the disease or condition in the subject.

In some embodiments, the present disclosure provides methods for treating or preventing a disease or condition such as cystic fibrosis, cancers, autoimmune diseases or conditions, or inflammatory diseases or conditions, comprising providing to the subject an effective amount of an Matrix Metalloproteinase 9 (MMP9) binding protein comprising an immunoglobulin heavy chain polypeptide, or functional fragment thereof, and an immunoglobulin light chain polypeptide, or functional fragment thereof, wherein the MMP9 binding protein specifically binds MMP9; and an effective amount of one or more additional therapeutic agents that is an oncology agent, thereby treating or preventing the disease or condition in the subject.

In some aspects, for treating an inflammatory or autoimmune disease, such as IBD, UC, Crohn's disease, cancer, or rheumatoid arthritis, the monotherapy of an anti-MMP9 antibody or antigen binding fragment thereof or the combination therapy of an anti-MMP9 antibody or antigen binding fragment thereof and an immune modulating agent is administered alone or with one or more additional therapeutic agents described herein.

Each of the agents in a combination therapy can be administered, via any suitable route, including any described herein, simultaneously (in the same composition or separately), or sequentially, in any order.

### Detection of MMP9

The present disclosure also contemplates methods of detecting MMP9 in a subject, e.g., to detect tumor or tumor-associated tissue expressing MMP9, or tissue or fluid or other biological sample associated with a disease as described herein, such as autoimmune or inflammatory disease. Thus, methods of diagnosing, monitoring, staging or detecting a tumor having MMP9 activity are provided.

Samples (e.g., test biological samples) from a subject (e.g., an individual suspected of having or known to have a tumor associated with MMP9 expression, or suspected of having or known to have another disease or condition, such as inflammatory or autoimmune disease as described herein), can be analyzed for MMP9 presence, absence, expression, and/or levels. For example, such samples can be collected and analyzed by detecting the presence or absence of binding of an MMP9 binding protein, such as an antibody or fragment as described herein, to substance (e.g., protein) in the sample. In some examples, the methods further include comparing the amount of binding detected to an amount of binding to a control sample, or comparing the detected level of MMP9 to a control level of MMP9. In some cases, the methods indicate the presence, absence, or severity of a disease or condition as described herein.

This analysis can be performed prior to the initiation of treatment using an MMP9 binding protein as described herein, or can be done as part of monitoring of progress of cancer treatment. In some embodiments, provided are methods of treatment, carried out by performing the detection assays and initiating, altering, or discontinuing treatment of the subject, for example, based on the results of the diagnostic assay. Such diagnostic analysis can be performed using any sample, including but not limited to tissue, cells isolated from such tissues, and the like. In some cases, the methods are performed on liquid samples, such as blood, plasma, serum, whole blood, saliva, urine, or semen. Tissue samples include, for example, formalin-fixed or frozen tissue sections.

Any suitable method for detection and analysis of MMP9 can be employed. Various diagnostic assay techniques known in the art can be adapted for such purpose, such as competitive binding assays, direct or indirect sandwich assays and immunoprecipitation assays conducted in either heterogeneous or homogeneous phases.

MMP9 binding proteins for use in detection methods can be labeled with a detectable moiety. The detectable moiety directly or indirectly produces a detectable signal. For example, the detectable moiety can be any of those described herein such as, for example, a radioisotope, such as 3H, 14C, 32P, 35S, or 125I, a fluorescent or chemiluminescent compound, such as fluorescein isothiocyanate (FITC), Texas red, cyanin, photocyan, rhodamine, or luciferin, or an enzyme, such as alkaline phosphatase, -galactosidase or horseradish peroxidase.

Detection can be accomplished by contacting a sample under conditions suitable for MMP9 binding protein binding to MMP9, and assessing the presence (e.g., level) or absence of MMP9 binding protein-MMP9 complexes. A level of MMP9 in the sample in comparison with a level of a reference sample can indicate the presence of a tumor or tumor-associated tissues having MMP9 activity. The reference sample can be a sample taken from the subject at an earlier time point or a sample from another individual.

In some aspects, MMP9 mRNA is detected, such as by hybridization, such as by chromogenic in situ hybridization (CISH). In some aspects, such detection methods are used when high levels of inflammatory cell-derived MMP9 obscure signal in a desired cell type by other detection method, e.g., by IHC, e.g., in tumor epithelia.

In certain embodiments, any of the methods of the present disclosure further comprise the step of determining whether the subject, or diseased cells obtained from the subject, overexpress MMP9 as compared to a control subject or non-diseased cells, e.g., non-diseased cells of the same cell type. In certain embodiments, the subject is provided with the MMP9 binding agent, alone or in combination with an immunomodulatory agent, if the subject overexpresses MMP9 but not if the subject does not overexpress MMP9.

The subject who is suitable to receive or who may benefit from the therapy and methods of the present disclosure may exhibit increased levels or activities of MMP9. Such subjects may be identified by screening or measuring the levels or expression of MMP9 protein which may be determined by commonly-used methods such as western blot, ELISA, mRNA hybridization, RNAseq, or single nucleotide polymorphism (SNP). Some SNPs have been correlated with increased MMP9 levels. The screening or identification of MMP9 levels/activities may also be used to monitor the patients' responses or treatment outcome.

### Pharmaceutical Compositions and Kits

Provided herein are compositions comprising: a pharmaceutically acceptable excipient, carrier or diluent; a Matrix Metalloproteinase 9 (MMP9) binding protein, e.g., comprising an immunoglobulin heavy chain polypeptide, or functional fragment thereof, and an immunoglobulin light chain polypeptide, or functional fragment thereof, wherein the MMP9 binding protein specifically binds MMP9; and one or more additional therapeutic agent, e.g., any of those described here, such as an immune modulating agent.

In another aspect of the disclosure, MMP9 binding proteins, as well as nucleic acid (e.g., DNA or RNA) encoding MMP9 binding proteins, can be provided as a pharmaceutical composition, e.g., combined with a pharmaceutically acceptable carrier or excipient. Such pharmaceutical compositions are useful for, for example, administration to a subject in vivo or ex vivo, and for diagnosing and/or treating a subject with the MMP9 binding proteins, such as in any of the therapeutic or diagnostic methods provided herein.

Pharmaceutically acceptable carriers or excipients are physiologically acceptable to the administered patient and retain the therapeutic properties of the antibodies or peptides with which it is administered. Pharmaceutically-acceptable carriers or excipients and their formulations are and generally described in, for example, Remington′ pharmaceutical Sciences (18th Edition, ed. A. Gennaro, Mack Publishing Co., Easton, Pa. 1990). One exemplary pharmaceutical carrier is physiological saline. Each carrier or excipient is “pharmaceutically acceptable” in the sense of being compatible with the other ingredients of the formulation and not substantially injurious to the patient.

Pharmaceutical compositions can be formulated to be compatible with a particular route of administration, systemic or local. Thus, pharmaceutical compositions include carriers, diluents, or excipients suitable for administration by various routes.

Pharmaceutical compositions can include pharmaceutically acceptable additives. Examples of additives include, but are not limited to, a sugar such as mannitol, sorbitol, glucose, xylitol, trehalose, sorbose, sucrose, galactose, dextran, dextrose, fructose, lactose and mixtures thereof. Pharmaceutically acceptable additives can be combined with pharmaceutically acceptable carriers and/or excipients such as dextrose. Additives also include surfactants such as polysorbate 20 or polysorbate 80.

The formulation and delivery methods will generally be adapted according to the site and the disease to be treated. Exemplary formulations include, but are not limited to, those suitable for parenteral administration, e.g., intravenous, intra-arterial, intramuscular, or subcutaneous administration, or oral administration. In one embodiment, the anti-MMP9 antibody or antigen binding fragment thereof, the composition or the formulation thereof is delivered by intravenous administration (which may be referred to as intravenous infusion). In some embodiment, the anti-MMP9 antibody or antigen binding fragment thereof, the composition or the formulation thereof is delivered by subcutaneous administration (which may be referred to as subcutaneous injection).

Pharmaceutical compositions for parenteral delivery include, for example, water, saline, phosphate buffered saline, Hank's solution, Ringer's solution, dextrose/saline, and glucose solutions. The formulations can contain auxiliary substances to approximate physiological conditions, such as buffering agents, tonicity adjusting agents, wetting agents, detergents and the like. Additives can also include additional active ingredients such as bactericidal agents, or stabilizers. For example, the solution can contain sodium acetate, sodium lactate, sodium chloride, potassium chloride, calcium chloride, sorbitan monolaurate or triethanolamine oleate. Additional parenteral formulations and methods are described in Bai (1997) J. Neuroimmunol. 80:65 75; Warren (1997) J. Neurol. Sci. 152:31 38; and Tonegawa (1997) J. Exp. Med. 186:507 515. The parenteral preparation can be enclosed in ampules, disposable syringes or multiple dose vials made of glass or plastic.

Pharmaceutical compositions for intravenous, intradermal or subcutaneous administration can include a sterile diluent, such as water, saline solution, fixed oils, polyethylene glycols, glycerin, propylene glycol or other synthetic solvents; antibacterial agents such as benzyl alcohol or methyl parabens; antioxidants such as ascorbic acid, glutathione or sodium bisulfite; chelating agents such as ethylenediaminetetraacetic acid; buffers such as acetates, citrates or phosphates and agents for the adjustment of tonicity such as sodium chloride or dextrose.

Pharmaceutical compositions for injection include aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). The carrier can be a solvent or dispersion medium containing, for example, water, ethanol, polyol (for example, glycerol, propylene glycol, and liquid polyetheylene glycol, and the like), and suitable mixtures thereof. Fluidity can be maintained, for example, by the use of a coating such as lecithin, by the maintenance of the required particle size in the case of dispersion and by the use of surfactants. Antibacterial and antifungal agents include, for example, parabens, chlorobutanol, phenol, ascorbic acid and thimerosal. Isotonic agents, for example, sugars, polyalcohols such as manitol, sorbitol, and sodium chloride may be included in the composition. The resulting solutions can be packaged for use as is, or lyophilized; the lyophilized preparation can later be combined with a sterile solution prior to administration.

Pharmaceutically acceptable carriers can contain a compound that stabilizes, increases or delays absorption or clearance. Such compounds include, for example, carbohydrates, such as glucose, sucrose, or dextrans; low molecular weight proteins; compositions that reduce the clearance or hydrolysis of peptides; or excipients or other stabilizers and/or buffers. Agents that delay absorption include, for example, aluminum monostearate and gelatin. Detergents can also be used to stabilize or to increase or decrease the absorption of the pharmaceutical composition, including liposomal carriers. To protect from digestion the compound can be complexed with a composition to render it resistant to acidic and enzymatic hydrolysis, or the compound can be complexed in an appropriately resistant carrier such as a liposome. Means of protecting compounds from digestion are known in the art (see, e.g., Fix (1996) Pharm Res. 13:1760 1764; Samanen (1996) J. Pharm. Pharmacal. 48:119 135; and U.S. Pat. No. 5,391,377, describing lipid compositions for oral delivery of therapeutic agents).

Compositions of the present disclosure can be combined with other therapeutic moieties or imaging/diagnostic moieties as provided herein. Therapeutic moieties and/or imaging moieties can be provided as a separate composition, or as a conjugated moiety present on an MMP9 binding protein.

Formulations for in vivo administration are generally sterile. In one embodiment, the pharmaceutical compositions are formulated to be free of pyrogens such that they are acceptable for administration to human patients.

Various other pharmaceutical compositions and techniques for their preparation and use will be known to those of skill in the art in light of the present disclosure. For a detailed listing of suitable pharmacological compositions and associated administrative techniques one can refer to the detailed teachings herein, which can be further supplemented by texts such as Remington: The Science and Practice of Pharmacy 20th Ed. (Lippincott, Williams & Wilkins 2003).

Pharmaceutical compositions can be formulated based on the physical characteristics of the patient/subject needing treatment, the route of administration, and the like. Such can be packaged in a suitable pharmaceutical package with appropriate labels for the distribution to hospitals and clinics wherein the label is for the indication of treating a disorder as described herein in a subject. Medicaments can be packaged as a single or multiple units. Instructions for the dosage and administration of the pharmaceutical compositions of the present disclosure can be included with the pharmaceutical packages and kits described below.

In one embodiment, a pharmaceutical composition is provided for treating or preventing an MMP9-associated disease or condition in a subject in need thereof, comprising: a pharmaceutically acceptable excipient, an anti-MMP9 antibody or antigen binding fragment thereof; and an immune modulating agent.

In one embodiment, a pharmaceutical composition is provided for treating or preventing cystic fibrosis in a subject in need thereof, comprising: a pharmaceutically acceptable excipient; and an anti-MMP9 antibody or antigen binding fragment.

In one embodiment, a pharmaceutical composition is provided for treating or preventing vasculitis in a subject in need thereof, comprising: a pharmaceutically acceptable excipient; and an anti-MMP9 antibody or antigen binding fragment.

In one embodiment, kits comprising a compound disclosed herein, or a pharmaceutically acceptable salt thereof, in combination with one or more (e.g., one, two, three, one or two, or one to three) additional therapeutic agents are provided.

Various aspects of the invention are further described and illustrated by way of the several examples which follow, none of which are intended to limit the scope of the invention.

### EXAMPLES

### Example 1: Activation of MMP9 Protein

Pro-MMP9 is cleaved by protease activators to remove the pro-domain and generate a catalytically active form of MMP9 (i.e. active MMP9). To examine whether endogenous (MMP3) and exogenous (Pseudomonas elastase) proteases or activators would cleave pro-MMP9 or activate MMP9, a cell-free assay was used. Pro-MMP9 was incubated at 37° C. with increasing concentrations of either active MMP3 or active Pseudomonas elastase (0.0034-200 nM). Both proteases activated MMP9 in a dose-dependent manner, as shown by the appearance of the active MMP9 fragment by Li-Cor Western blot and increase in gelatinolytic activity (data not shown). The activation of MMP9 by MMP3 and Pseudomonas elastase was inhibited by AB0045 (data not shown). MMP9 auto-activation was not observed in vitro. The result indicates that AB0045 inhibits the activation of MMP9 as an MMP9-specific protease inhibitor.

Additional antibodies specific to active MMP9 were generated. One antibody (Active AB) was used for additional studies. The heavy and light chain sequences of Active AB are as follows:

Active AB Heavy Chain:
(SEQ ID NO: 59)
QSVEESGGRLVTPGTPLTLTCTASGFTISSYHMTWVRQAPMKGLEWI
GTISSSGSTYYASWAKGRFTISKTSSTTVDLKITSPATEDTATYFCA
RSVPGDSSGEIWGRGTLVTVSSGQPKAPSVFPLAPCCGDTPSSTVTL
GCLVKGYLPEPVTVTWNSGTLTNGVRTFPSVRQSSGLYSLSSVVSVT
SSSQPVTCNVAHPATNTKVDKTVAPSTCSKPTCPPPELLGGPSVFIF
PPKPKDTLMISRTPEVTCVVVDVSQDDPEVQFTWYINNEQVRTARPP
LREQQFNSTIRVVSTLPIAHQDWLRGKEFKCKVHNKALPAPIEKTIS
KARGQPLEPKVYTMGPPREELSSRSVSLTCMINGFYPSDISVEWEKN
GKAEDNYKTTPAVLDSDGSYFLYSKLSVPTSEWQRGDVFTCSVMHEA
LHNHYTQKSISRSPGK
Active AB Light Chain:
(SEQ ID NO: 60)
AQVLTQTASPVSAAVGGTVTINCQSSQSVYNKNWLAWYQQKPGQPPK
RLIYSASTLDSGVSSRFKGSGSGTQFTLTISGVQCDDAATYYCQGEF
SCSRGDCSAFGGGTEVVVQGDPVAPTVLIFPPSADLVATGTVTIVCV
ANKYFPDVTVTWEVDGTTQTTGIENSKTPQNSADCTYNLSSTLTLTS
TQYNSHKEYTCKVTQGTTSVVQSFNRGDC

The reagent antibodies L51/82 (Biolegend), which recognizes total amounts of MMP9 regardless of activation state, and Abcam 76003 were also used. Specificity of Active AB was determined by Western blot analysis and immunohistochemistry (IHC).

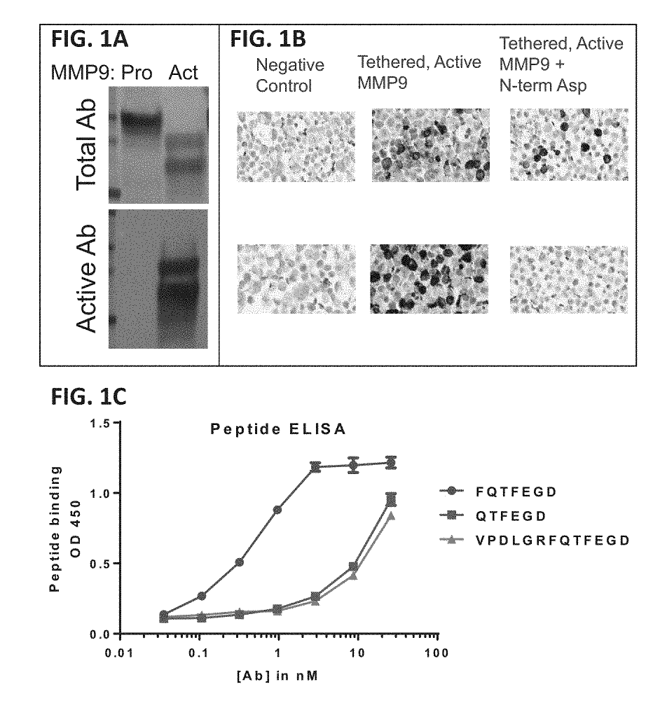

The results showed that both antibodies Active AB and L51/82 bound to active MMP9 (FIG. 1A). Addition of an N-terminal aspartic acid reduced the binding of Active AB to active MMP9 protein and did not affect the binding of the Abcam 76003 to the MMP9 protein (FIG. 1B). Specificity of the Active AB was further shown by ELISA with peptides of the neo-epitope (FQTFEGD) (SEQ ID NO: 56), a fragment of the neo-epitope (QTFEGD) (SEQ ID NO: 57), and the total fragment of cleavage site (VPDLGRFQTFEGD)(SEQ ID NO: 58). The binding of Active AB to the neo-epitope (FQTFEGD) occurred at low concentrations of antibody, and the binding of Active AB to the neo-epitope fragment (QTFEGD) and the total cleavage site (VPDLGRFQTFEGD) occurred at increased concentrations of antibody (FIG. 1C).

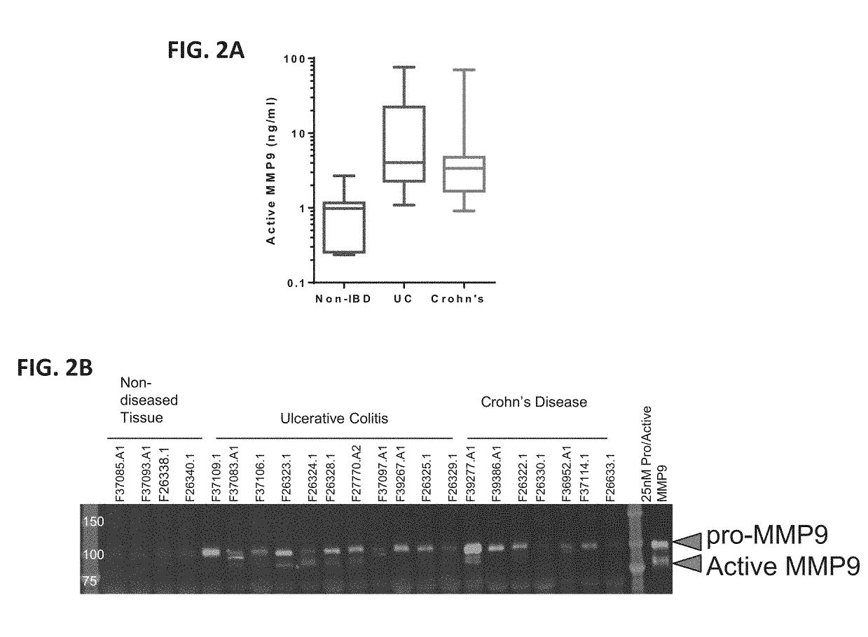

Moreover, the binding of Active AB to total and active MMP9 in human tissues was assessed using colon lysates from ulcerative colitis (UC) and Crohn's disease patients. Results of Li-Cor Western blot showed that Active AB specifically bound to active MMP9 and differentiated the presence of pro- and active MMP9 in human samples (FIG. 2B).

### Example 2: Active MMP9 in Chronic Myeloid Inflammatory Disease Tissue

To quantify endogenous or naive MMP9 activity (proteolysis of substrate peptide), an MMP9 assay using the GE MMP-9 Biotrak assay kit was developed. Plates were coated with a monoclonal antibody specific for human MMP9 which recognized epitopes unrelated to the cleavage site. APMA was omitted to ensure endogenous or native, not induced, MMP9 activity was examined. The endogenous or naïve MMP9 activity represented the MMP9 level and/or activity in the real disease state. After sample addition and incubation at 4° C. overnight, plates were washed and incubated with a substrate peptide conjugated to a fluorescent dye and a quencher. Cleavage of the substrate peptide removes the quencher and allows the dye to fluoresce, indicating the presence of active MMP9.

The above assay was used to examine the MMP9 activity in colon tissue lysates from ulcerative colitis (UC) and Crohn's disease patients. The results showed that MMP9 activity in samples from UC and Crohn's disease patients was increased compared to those of non-inflammatory bowel disease (IBD) patients (FIG. 2A). Li-Cor Western Blots was used to further analyze the lysates from UC, Crohn's disease, and non-diseased control tissues. The results showed the levels of both pro- and active MMP9 in UC and Crohn's disease tissues were increased compared to those of non-diseased tissues (FIG. 2B).

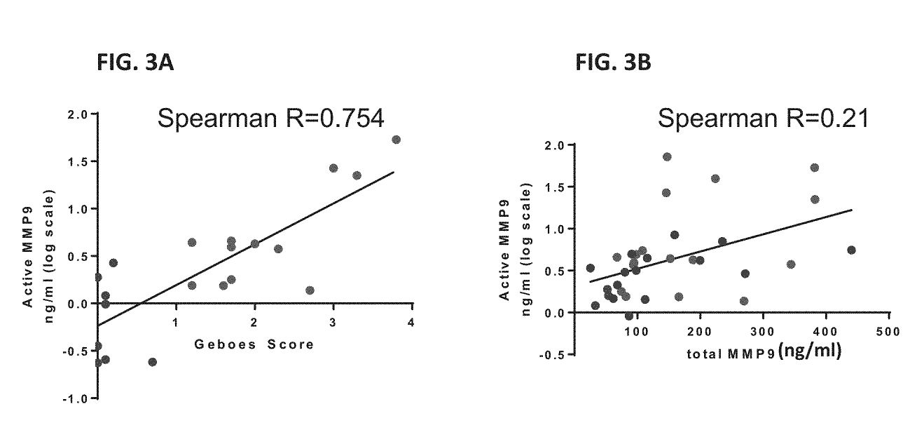

After detection of both pro- and active MMP9 in diseased tissue lysates, correlation analyses between both forms of the protein and disease score were determined with matched FFPE samples analyzed histologically. As shown in FIG. 3A, there was between the active MMP9 concentration and Geboes disease score (Spearman correlation=0.754). Correlation was reduced between active MMP9 and total MMP9 for non-IBD, UC and Crohn's disease state (Spearman correlation=0.21) FIG. 3B. This indicates that active MMP9, not total MMP9, correlates with UC histological disease score.

Endogenous or naive MMP9 activity of diseased tissue from UC and Crohn's disease patients was examined. The average levels of active MMP9 in the tissue of UC and Crohn's disease patients were 14.8 ng/mL and 8.3 ng/mL, respectively. These levels are increased compared to those of tissue from inflammatory bowel disease and normal tissue (<1 ng/mL).

Hidradenitis suppurativa (HS) is a prevalent chronic inflammatory skin condition characterized by fistulae formation. IHC analyses of tissue from HS patients showed increased staining for active MMP9. Fistulae were also characterized by significant staining for myeloperoxidase (MPO), indicative of active neutrophil infiltration. Moderate staining for the macrophage marker, ionized calcium binding adaptor molecule 1 (IBA1), the B-cell marker CD20, and the T-cell marker CD3 were also detected (data not shown). This staining pattern indicated an active inflammatory state characterized by MMP9 expression and myeloid cell infiltration.

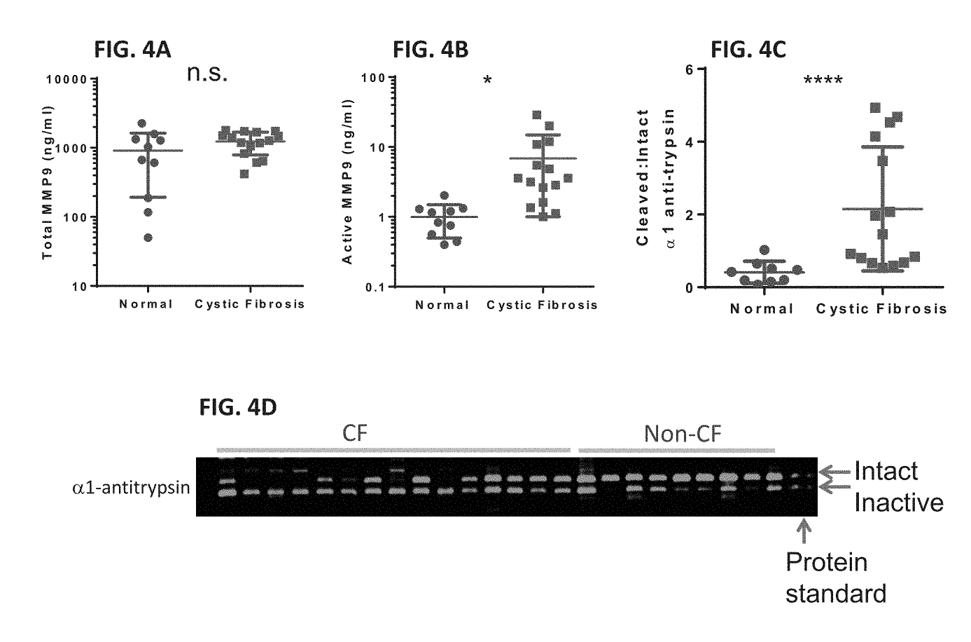

Similar to IBD and HS, cystic fibrosis (CF) is characterized by aberrant myeloid inflammation. Chronic inflammation is hypothesized to result in pathologic lung remodeling and decline in lung function in CF patients. As shown in FIG. 4A, similar levels of total MMP9 were detected in lung lysates from non-CF and CF patients. Measurements of endogenous active MMP9 showed the increase levels of active MMP9 in samples from CF patients as compared to those of normal controls (FIG. 4B, * p=0.03). Also, samples from CF patients also exhibited elevated ratios of inactive (cleaved) vs. active (intact) α1-antitrypsin, indicating an increase in levels of inactive α1-antitrypsin in CF lung tissue (FIG. 4C, **** p=0.0001). Ratios of intact: inactive α1-antitrypsin were determined by quantitative Li-Cor Western blot. Lysates from CF patients showed decreased intact (active) al-antitrypsin and increased levels of cleaved (inactive) α1-antitrypsin (FIG. 4D). These results indicated that active MMP9 levels correlate with CF and decreased levels of active α1-antitrypsin.

Together, the results suggest that active MMP9 may be associated with multiple chronic myeloid inflammatory diseases and that levels of active MMP9 may correlate with disease severity, indicating a potential role for MMP9 as a biomarker in myeloid inflammatory diseases and as an active player in the inflammatory milieu of the diseased tissue.

### Example 3: MMP9 Activity Correlates with Inactivation of α1-Antitrypsin in Cystic Fibrosis Lung Tissue

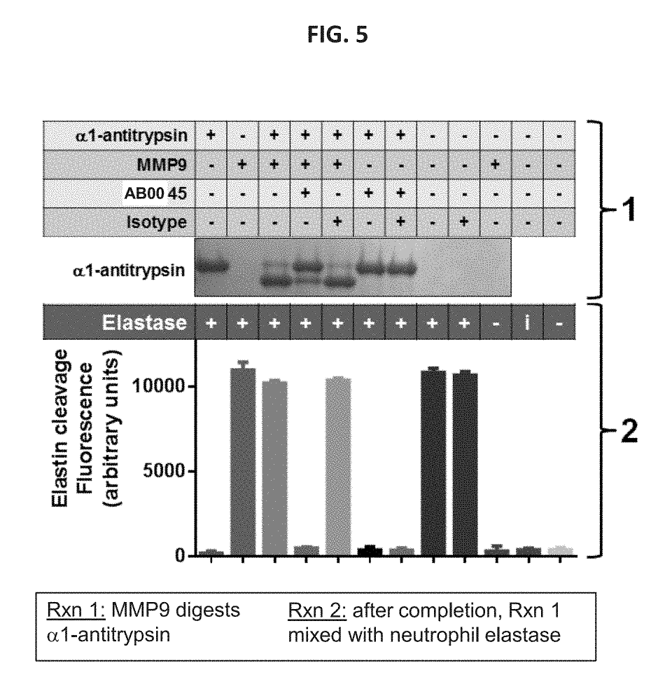

It is known that α1-antitrypsin inhibits human neutrophil elastase (FINE), which is a key mediator of lung destruction. Loss of function mutations in α1-antitrypsin are associated with decreased lung function. The ability of MMP9 to directly inactivate α1-antitrypsin was assessed in vitro. In reaction 1 (Rxn1), intact α1-antitrypsin was incubated with active MMP9 in the presence or absence of AB0045. Cleavage of α1-antitrypsin was assessed by Western blot (FIG. 5, panel 1). In the presence of active MMP9 alone, α1-antitrypsin was cleaved from the active form to the inactive form. This inactivation was inhibited by the addition of AB0045 and unaffected by the addition of an isotype control. The digests from Rxn1 were then incubated with neutrophil elastase, a key mediator of lung destruction, and its substrate, elastin (FIG. 5, Panel 2). The ability of digests from Rxn1 to inhibit neutrophil elastase was measured by elastin cleavage fluorescence in reaction 2 (Rxn2). Intact α1-antitrypsin inhibited neutrophil elastase, indicated by a lack of elastin fluorescence. MMP9-inactivated al-antitrypsin did not inhibit the cleavage of elastin by neutrophil elastase. Addition of AB0045, resulting in subsequent inactivation of MMP9, was sufficient to prevent downstream elastin cleavage by neutrophil elastase. As a control, the elastase inhibitor N-methoxylsuccinyl-Ala-Ala-Pro-Val-chloromethyl ketone also inhibited HNE (shown as “i” in FIG. 5, panel 2). These results indicate that AB0045 may prevent the inactivation of α1-antitrypsin and may lead to restore α1-antitrypsin function and reduce HNE activity.

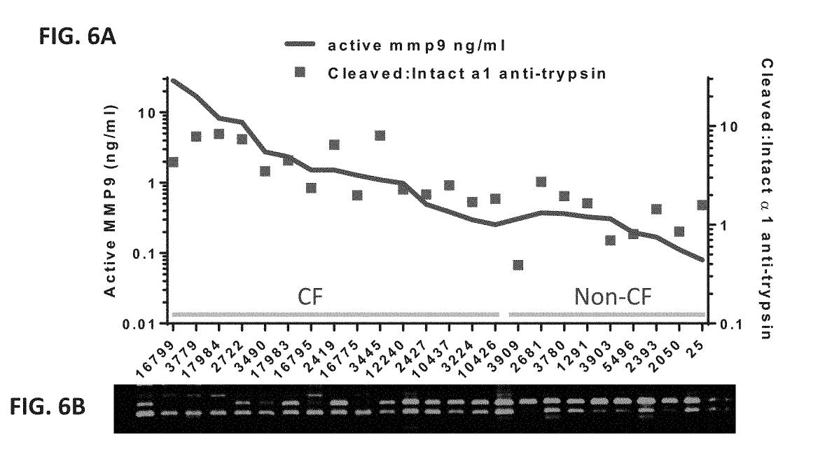

Further, correlation between MMP9 activity and α1-antitrypsin inactivation was assessed in vivo. Levels of active MMP9 and ratios of cleaved vs. intact α1-antitrypsin were compared between lung lysates from CF and non-CF patients (FIG. 6A). Relative ratios of cleaved vs. intact α1-antitrypsin were visualized by Western blots of α1-antitrypsin (FIG. 6B). Spearman correlations between MMP9 activity and α1-antitrypsin cleavage were calculated for all patients (Spearman correlation=0.85, p<0.0001) and only CF patients (Spearman correlation=0.7, p<0.0045).

These data indicate that MMP9 may directly inactivate α1-antitrypsin to allow function of inflammatory proteases such as neutrophil elastase. Further, the activity of MMP9 correlates with inactivation of α1-antitrypsin in CF lung tissue, suggesting a mechanism by which MMP9 may mediate inflammation in CF.

### Example 4: MMP9 Inhibition in the Xenograft Model

This study examines the effect of MMP9-specific inhibition in the xenograft model. Fragments of subcutaneous tumors derived from a human colorectal cancer cell line (HCT-116) were surgically implanted into the colon in nude mice and allowed to grow to ˜100 mm³ in volume prior to treatment initiation. Mice were treated with vehicle, isotype IgG (control), or a 1:1 mixture of AB0041 and AB0046 (anti-mouse MMP9 and anti-human MMP9, respectively) (m+h). Treatments were administered intraperitoneally at 30 mg/kg total antibody (15 mg/kg of each AB0041 and AB0046) twice a week. In the m+h group, mice were also pre-administered with AB0046 at 50 mg/kg on the first day of the treatment.

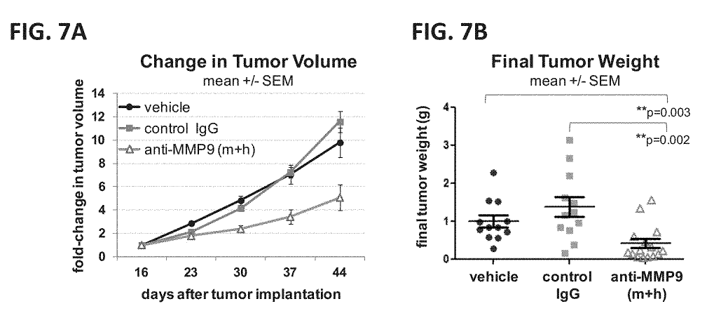

Primary tumor sizes were measured once a week using a caliper. Caliper-based size estimates were obtained by measuring the perpendicular minor dimension (W) and major dimension (L) of the palpated tumor. Approximate tumor volume was calculated by the formula (W²×L)/2. The non-parametric Mann-Whitney rank sum test was used to determine p values. Treatment with the antibody cocktail decreased change in tumor volume (FIG. 7A) and decreased final tumor weight (FIG. 7B). Immunohistochemistry (IHC) analysis was also performed on tumors from vehicle-treated mice and demonstrated production of MMP9 by tumor cells at lower levels compared to MMP9 from stromal sources such as resident macrophages, fibroblasts, and epithelial cells (data not shown).

Tumor sections from isotype and αMMP9 treated mice were also visualized via 2^{nd} harmonic microscopy. The sections were stained with picro-sirius red (PSR) to visualize collagen deposition, and αKi67 antibodies were used to visualize cellular proliferation by IHC analysis (data not shown). Tumor sections from mice treated with αMMP9 antibodies showed an increased degree of fibrillar collagen remodeling adjacent to the tumor. These results indicate that targeting MMP9 with a cocktail of human-specific and mouse-specific monoclonal antibodies in a mouse xenograft model reduced growth of the primary tumor and remodeled fibrillar collagen.

### Example 5: Treatment of Rheumatoid Arthritis

A Phase 1, double-blind, randomized, placebo-controlled study was conducted for RA patients (subjects). Subjects were randomized in a 4:1 ratio to receive an intravenous (IV) infusion of AB0045 at 400 mg or matched placebo every 2 weeks for a total of 3 infusions (Days 1, 15, and 29). Subjects participated in the study for up to 117 days. The screening visits were conducted a maximum of 15 days before the first infusion. Subjects exhibited a mean C-reactive protein (CRP) value during screening ≧8 mg/L and did not received other concomitant RA treatments within 1-12 months prior to or during the study.

The Disease Activity Score (DAS) served as a key clinical endpoint for this study. Using the DAS-28 CRP score, subjects were classified by level of RA disease activity at baseline (>5.1 severe, >3.2≦5.1 moderate, mild >2.6<3.2, and remission <2.6) (Wells et al, Annals of the rheumatic diseases 2009; 68 (6):954-60). Among subjects treated with AB0045 (N=15), baseline disease activity levels were categorized as severe for 13 subjects (87.7%), and moderate for 2 subjects (13.3%). No subjects were considered mild or in remission at baseline. At Day 43, after receiving 3 doses of IV AB0045, disease activity was categorized as severe for 3 subjects (20.0%), moderate for 8 subjects (53.3%), low for 3 subjects (20.0%), and 1 subject was in remission (6.7%). Among placebo subjects (N=3), the distribution of disease activity at Baseline was severe for 2 subjects (66.7%) and moderate for 1 subject (33.3%). No subjects were considered mild or in remission. In contrast to AB0045 treated subjects, at Day 43 the placebo group had no subjects with mild disease activity or remission. All 3 placebo subjects at Day 43 exhibited moderate disease activity. These clinical improvements and changes in additional RA disease activity measures are shown in Table 2.

TABLE 2
Subjects with 50% improvement at Day 43

| | | | Number of Subjects with 50% Improvement | | | | | | | | | | | |
|---|---|---|---|---|---|---|---|---|---|---|---|---|---|---|
| | Clinical | | | | | | | | Subject | | Physician | | | |
| | Assessments | | | | | | Subject | | Global | | Global | | | |
| | DAS28-CRP | | Swollen | | Tender | | Pain | | Assessment | | Assessment | | | |
| | (Mean Δ From | | Joint | | Joint | | Score | | of Health | | of Health | | | |
| | Baseline at | | Counts | | Counts | | (VAS, | | (VAS, | | (VAS, | | CRP | |
| Subjects | Day 43 | | (28-joints) | | (28-joints) | | 0-100) | | 0-100) | | 0-100) | | (mg/L) | |
| AB0045 | −1.35 | (1.393) | 9 | (60%) | 7 | (47%) | 7 | (47%) | 6 | (40%) | 6 | (40%) | 4 | (27%) |
| (N = 15) | | | | | | | | | | | | | | |
| Placebo | −077 | (0.570) | 0 | (0%) | 1 | (33%) | 0 | (0%) | 0 | (0%) | 0 | (0%) | 1 | (33%) |

(N = 3)

These clinical improvements occurred despite a lack of major change in CRP values. Among subjects treated with AB0045 during the study (n=15), the mean (SD) values at baseline and Day 43 were 37.21 (18.688) mg/L and 21.10 (18.977) mg/L, respectively, a mean decrease of 6.11 (22.577) mg/L. Among subjects treated with placebo (n=3), the mean CRP values at baseline and Day 43 were 16.57 (11.153) mg/L and 12.46 (10.572) mg/L, respectively, a mean decrease of 4.12 (5.922) mg/L. These study results showed that AB0045 was well tolerated during the study and that AB0045 may be beneficial in patients with RA (Table 2).

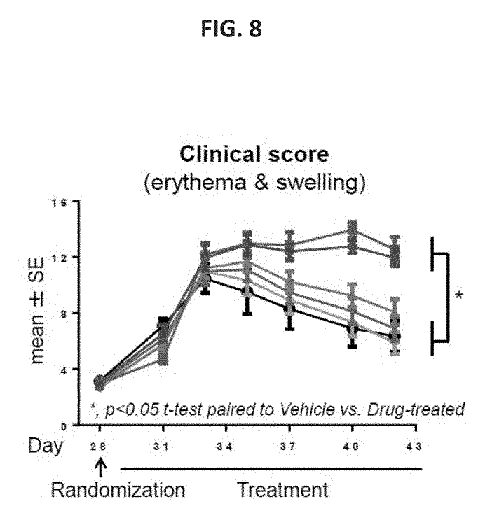

Additionally, the combination of an anti-MMP9 agent (AB0046) and an anti-TNF agent (Enbrel®) was evaluated in a murine collagen-induced arthritis (CIA) model. In this chronic model of advanced disease, therapies (vehicle, AB005123 control IgG, methotrexate, AB0046, Enbrel®, or combination) were administered after an average clinical score of >2 was reached (Day 28) and continued through Day 43. Score was determined using established methods on a scale of 0-4.0 (in 0.5 unit increments) and reflects increasing degrees of erythema and swelling across ankles/wrists and paws. All 4 paws were scored, thus each mouse has a theoretical maximum score of 16. The mean represents the treatment group average at each noted time point. Treatment with AB0046 and Enbrel®, alone or in combination, resulted in improvement with respect to scores (FIG. 8 *p<0.05 by t-test for vehicle or control compared to drug treated). Area under the curve (AUC) reflects cumulative clinical score over duration of treatment for each therapy. As illustrated by the AUC for the course of treatment of the study, treatment with a combination of AB0046 and Enbrel® (AUC=107.6) resulted in an improvement as compared to vehicle (AUC=145.3), control IgG (AUC=145.9), AB0046 alone (AUC=121.0), or Enbrel® alone (AUC=114.9). Similar results were shown in body weight and histopathology.

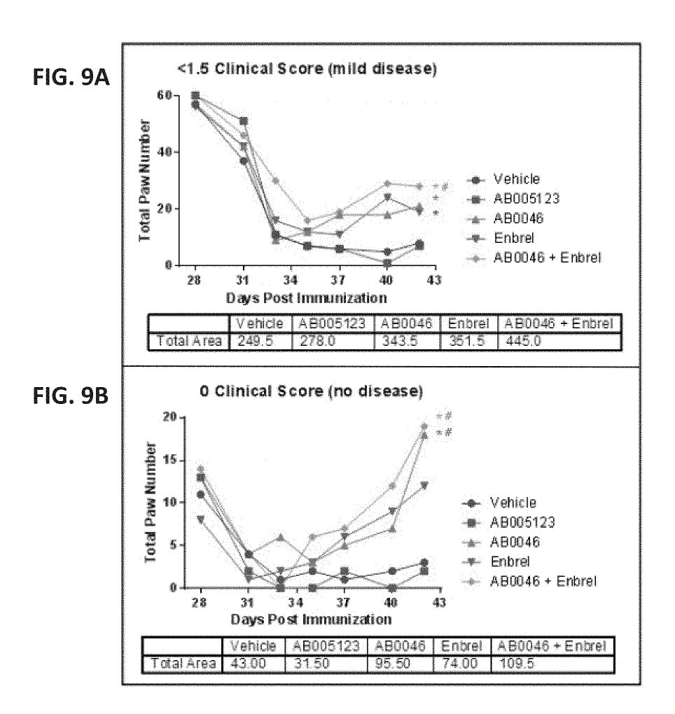

Further, the analysis evaluating the number of limbs scored with mild or no disease at the end of treatment showed that the combination therapy resulted in improvement when compared to individual agent (mild disease, see FIG. 9A, *p<0.05 paired t-test compared to vehicle, # p<0.05 paired t-test to Control IgG, AB0046, or Enbrel®; FIG. 9B, * p=0.052 paired t-test to vehicle, # p<0.05 paired t-test to Control IgG). Furthermore, analysis of complete blood count at the end of study revealed no abnormalities in any treatment group. These results indicate that the addition of anti-MMP9 to anti-TNF therapy or the combination therapy of anti-MMP9 to anti-TNF may potentially provide an increased therapeutic benefit or efficacy.

A Phase 2 trial in subjects with moderate to severe RA despite stable therapy with a TNF inhibitor is conducted to further evaluate the efficacy, safety, and pharmacokinetics of AB0045. Subjects with moderate to severe RA are enrolled and randomized in a 1:1:1 blinded fashion to receive either 300 mg or 150 mg of subcutaneous (SC) AB0045 weekly, or SC placebo weekly for 12 weeks in addition to their current SC administration of a TNF inhibitor. Subjects are stratified by disease activity with those with high disease activity defined as DAS-28-CRP>5.1 and those with moderate disease activity defined as a DAS-28-CRP≧3.2 and ≦5.1. In addition, subjects are stratified by prior treatment (1 to 2 treatments or 3 or more treatments) including the TNF inhibitor being administered during screening.

### Example 6: Combination Treatment

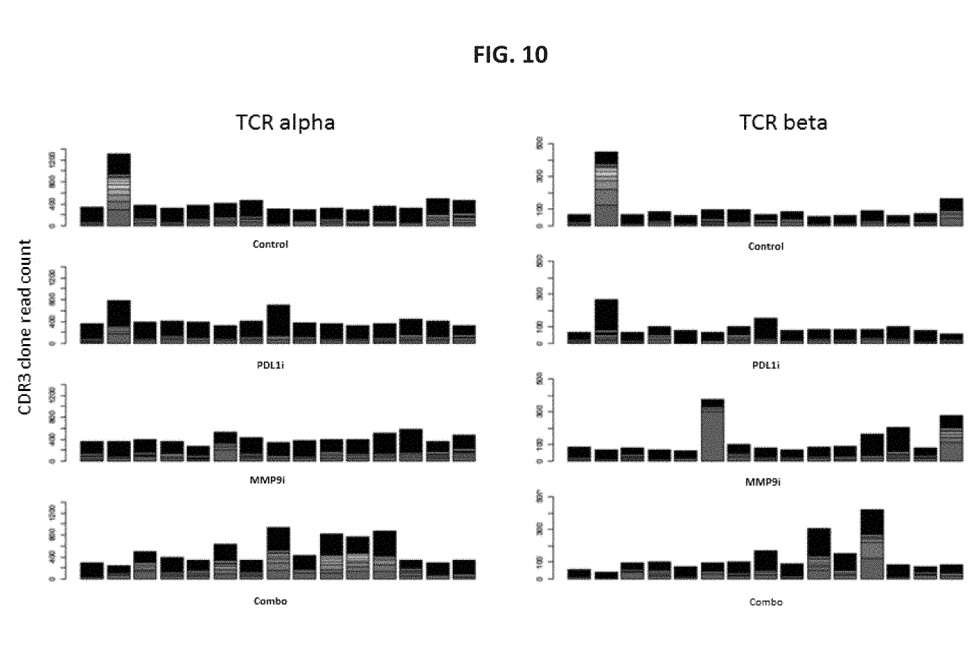

Bulk tumors from the mice injected with the HC11-NeuT breast cancer cell line were analyzed by RNA-Seq, regression, and Gene Set Enrichment Analysis (GSEA). The expression profiles were different between the mice treated with an anti-MMP9 antibody and those treated with an anti-PD-L1 antibody, yet there were overlapping pathways. The results showed that immunomodulatory pathways were upregulated in the group treated with both anti-MMP9 and anti-PD-L1 (data not shown). As several of the affected immunomodulatory pathways centered on TCR signaling, T cell diversity was measured by assessing CDR3 sequence diversity by application of MiTCR/MiXCR analysis to the RNAseq data. The analysis revealed that the combination of αMMP9 and αPDL1 treatment groups resulted in increased overall CDR3 counts, suggesting improved T cell diversity (FIG. 10). This study suggests that the combination therapy of anti-MMP9 and anti-PD-L1 may potentially increase or enhance the overall immune response to cancer antigens which may lead to anti-cancer responses and reduction in tumor growth.

### Example 7: Treatment of Cystic Fibrosis Patients

This study evaluates the effect of AB0045 on pre-bronchodilator forced expiratory volume in 1 second (FEV₁) in subjects with cystic fibrosis (CF) after 8 weeks of treatment. The primary outcome measure is the absolute change in pre-bronchodilator FEV₁ percent predicted from baseline to week 8. The secondary outcome measures are the safety evaluations, primary pharmacokinetics (PK) parameters, the absolute change in post-bronchodilator FEV₁ percent predicted from baseline to week 8, the relative change in pre-bronchodilator FEV₁ percent predicted from baseline to week 8, and the relative change in post-bronchodilator FEV₁ percent predicted from baseline to week 8. Safety evaluations are assessed by adverse events (AEs), concomitant medications, clinical laboratory tests, vital signs, and anti-drug antibodies (ADA) data. Primary PK parameters include Cmax (maximum concentration of drug), Tmax (the time of Cmax), Clast (last observable concentration of drug), Tlast (time of Clast), and AUClast (total amount of drug absorbed by the body), as applicable.

This study has two parts. Part 1 has a treatment arm in which participants receive 600 mg of AB0045 given subcutaneously once weekly for 8 weeks. Part 1 has a placebo arm in which participants receive a placebo to match AB0045 once weekly for 8 weeks. Part 2 has two treatment arms, one in which participants receive 300 mg of AB0045 given subcutaneously once weekly for 8 weeks, and one in which participants receive 150 mg of AB0045 given subcutaneously once weekly for 8 weeks. Part 2 has a placebo arm, in which participants are given placebo to match AB0045 once weekly for 8 weeks. Some inclusion criteria of the study includes (1) Pre-bronchodilator FEV₁≧40% and ≦80% of predicted at Screening, (2) two pre-bronchodilator spirometry measures taken at least 4 days apart (one during Screening, one at Baseline) using the sponsor provided central spirometry equipment must meet the following 2 criteria: (i) the relative difference of FEV₁(L), calculated as the absolute value of [(first FEV₁−second FEV₁)/first FEV1]×100 should be <12%, and (ii) the absolute difference in FEV₁ should be <200 ml.

### Example 8: Treatment of Giant Cell Arteritis

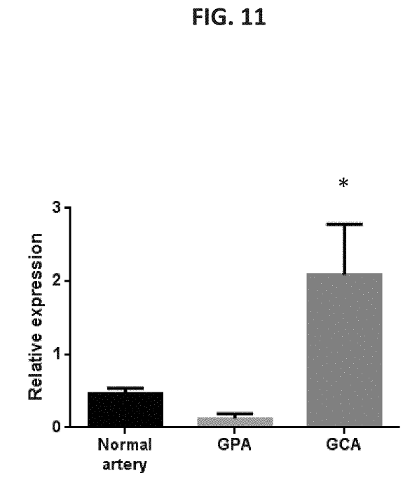

Vasculitis is inflammation of blood vessel walls. Giant cell arteritis (GCA) is a form of vasculitis that typically affects the network of small blood vessels that supply larger arteries. This study examined whether MMP9 would be involved in vessel wall inflammation, remodeling and myofibroblast mobilization/proliferation and the potential effects of MMP9 inhibition on anti-inflammatory activities in large vessel vasculitis. Analysis of mRNA expression revealed that MMP9 expression was increased in GCA arteries compared to normal arteries and arteries affected by granulomatosis with polyangiitis (Wegener's, GPA) (FIG. 11, * p<0.05).

A murine model of vasculitis was used to determine the potential effects of MMP9 inhibition on the pathology of vasculitis. Normal temporal or axillary arteries were engrafted into NSG immune deficient mice. After 7 days (i.e. Day 7 of the study), 20×10⁶ peripheral blood mononuclear cells (PBMCs) from GCA patients were transferred into the chimeric mice. Ten days after transfer, vasculitis of the engrafted human arteries was evident with tissue-infiltrating cells populating the vessel wall lesions. No vasculitis was observed when PBMCs from normal human controls were transferred. Dexamethasone injections served as a positive control and vehicle injections as negative controls. The model may be useful in evaluating the potential effects in preventing (prior to disease development) and/or treating the disease (after the disease is developed or established).

An anti-MMP9 antibody AB0045 or a control isotype Ig antibody (Isotype) was introduced during the beginning stages of vasculitis (Day 7, the same day as PBMC reconstitution) or during established vasculitis (Day 14, 7 days post PBMC reconstitution). Treating the chimeric mice at Day 7 is designed to target the early phase of the disease and to prevent vasculitic infiltrates from taking root, while therapeutic intervention at Day 14 mimics treatment of steady-state vasculitis. In each study, mice were engrafted with segments from the same artery and received an adoptive transfer of PMBC from the same patient, so that the vasculitis was comparable in each of the treatment arms. The antibodies were given every other day for a total of 3 times. Samples were collected at either Day 14 (for early phase) or Day 21 (for steady state vasculitis).

The effect of MMP9 inhibition on suppression of vasculogenic T cell functions in the vessel wall lesions was examined. Human arterial grafts were explanted at the end of the treatment period and analyzed for IL-6, TNF-α, IFN-γ, IL-1β, T cell receptor, IL-17, and IFN-γ expression by RT-PCR and immunohistochemistry. Tissue histology slides were stained with hematoxylin and eosin stain (H&E) to visualize artery architecture and cellular infiltrate. The group that received AB0045 treatment exhibited reduced cellular infiltrate into the artery wall, prevented arterial wall thickening, and maintained the integrity of vessel wall when compared to the Isotype treated group (data not shown). These data indicate that inhibition of MMP9 may play a role to maintain the artery integrity and may reduce the inflammatory responses.

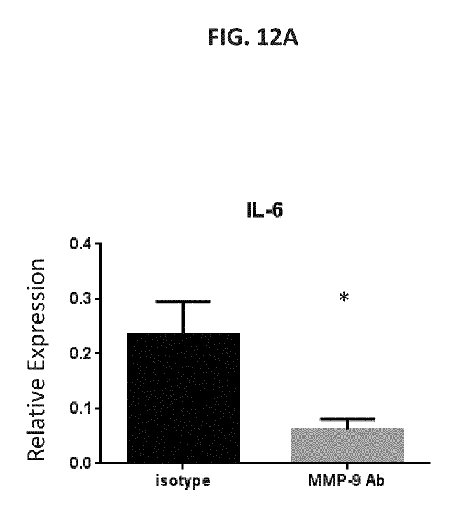

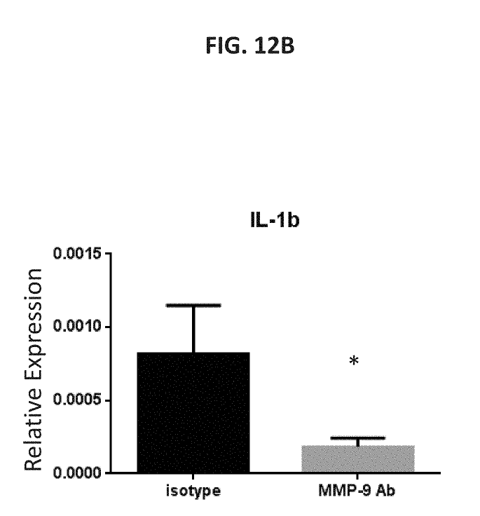

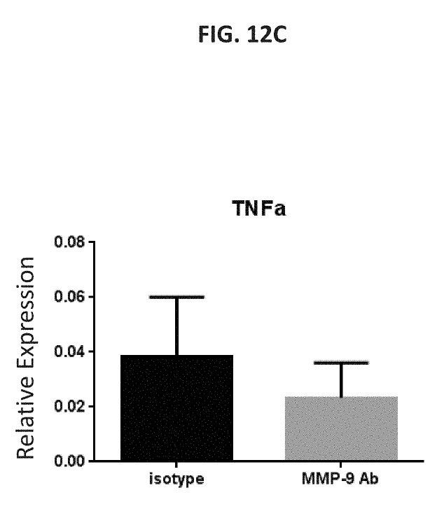

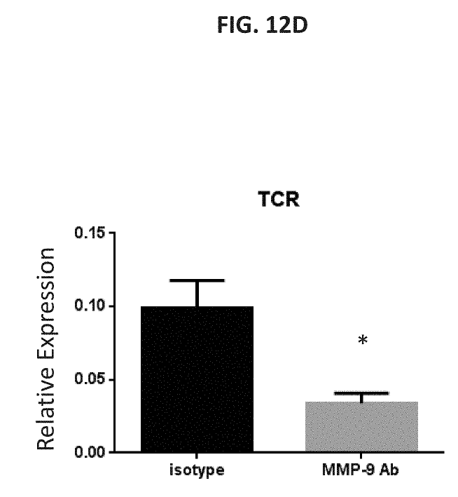

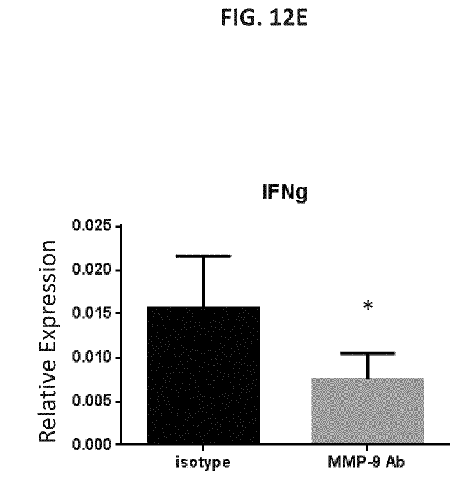

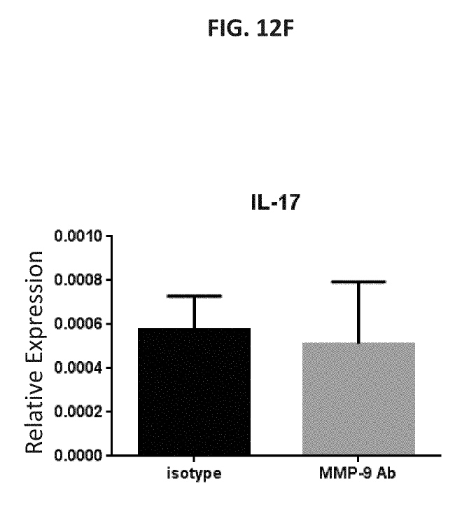

Six different arteries from the mice in Isotype or AB0045 groups were analyzed for expression of inflammatory cytokines by qPCR. Arteries from AB0045 treated group exhibited decreased IL-6 expression (FIG. 12A, * p<0.05) and decreased IL-1β expression (FIG. 12B, * p<0.05), and decreased TNF-α expression (FIG. 12C). These data indicate that AB0045 inhibits inflammatory cytokine expression in human arteries.

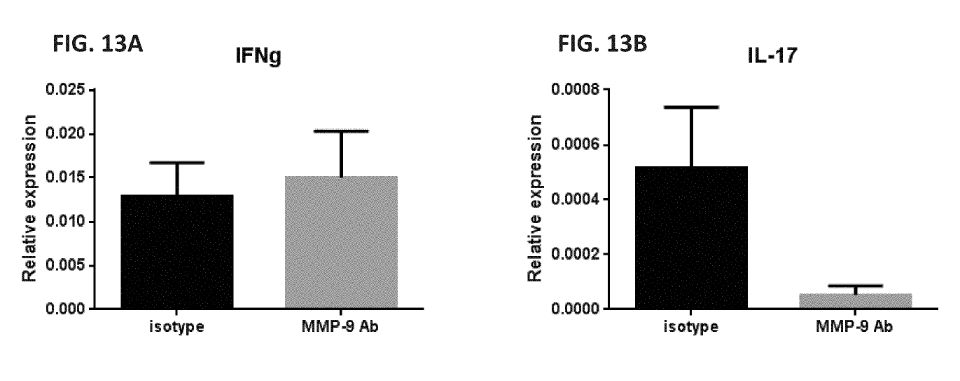

In addition, AB0045 treatment reduced TCR expression in the vessel walls (FIG. 12D, * p<0.05), suggesting an inhibition of T cell infiltrate after vasculitis induction. Furthermore, αMMP9 treatment reduced IFN-γ expression (FIG. 12E, * p<0.05), suggesting that αMMP9 treatment may abrogate Th1-committed T cells. The effect on T cell polarization may be specific, as the group that received AB0045 exhibited similar levels of IL17 expression in the established vasculitis study (FIG. 12F). Treating with AB0045 during early disease initiation (starting treatment on the same day as the PBMC adoptive transfer or Day 7 of the study) resulted in no effects on IFN-γ expression (FIG. 13A) and decreased IL-17 expression (FIG. 13B). These data suggest that αMMP9 modulates the inflammatory response during vasculitis.

### Example 9: Treatment of Adults with Unresectable or Recurrent Gastric or Gastroesophageal Junction Adenocarcinoma

This study evaluates the potential efficacy of an anti-MMP9 antibody (AB0045) in combination with a PD-1 inhibitor (Nivolumab) in treating unresectable or recurrent gastric or gastroesophageal junction (GEJ) adenocarcinoma. The subjects that receive benefit from the treatment have locally advanced or metastatic adenocarcinoma of the stomach or the GEJ which is histologically confirmed inoperable and who have received one prior line of therapy.

The following screening criteria is used for this study: medical history review, physical exam, vital signs, 12-lead ECG (electrocardiogram), ECOG (Eastern Cooperative Oncology Group) performance status, prior/concomitant medication review, chemistry, hematology, and coagulation, adverse event (AE) assessment, archival or recent biopsy FFPE (formalin-fixed paraffin embedded) tissue block collection, and computed tomography (CT) or magnetic resonance imaging (MRI). Additional screening criteria include baseline tumor lesions and archival tumor tissue adequate for PD-1 immunohistochemical stratification test.

Approximately 120 subjects are randomized to receive treatment which occurs every 2 weeks. Subjects who meet eligibility undergo CT scans or MRI every 8 weeks. Starting on Day 1, subjects randomized to Arm A (AB0045+nivolumab) receive 800 mg AB0045 via intravenous infusion (IV) infusion over approximately 30 minutes in advance of nivolumab 3 mg/kg via IV infusion over approximately 60 minutes on Day 1 and every 2 weeks thereafter. Subjects randomized to Arm B (nivolumab only) receive nivolumab 3 mg/kg via IV over approximately 60 minutes on Day 1 and every 2 weeks thereafter. Treatment continues every 2 weeks in the absence of disease progression or toxicity, and may last for up to 2 years.

The arms and interventions of the study are described in Table 3.

TABLE 3
Arms and interventions

| Arms | Assigned Interventions |
|---|---|
| Arm A | AB0045 |
| AB0045 + Nivolumab | 800 mg administered via intravenous |
| for up to 2 years | (IV) infusion every 2 weeks |
| | Nivolumab |
| | 3 mg/kg administered via intravenous |
| | (IV) infusion every 2 weeks |
| Arm B | Nivolumab |
| nivolumab for up | 3 mg/kg administered via intravenous |
| to 2 years | (IV) infusion every 2 weeks |

After treatment, the study safety, efficacy, and pharmacokinetics is determined at various time points, such as 12 weeks, 48 weeks, 96 weeks, 1 year or 2 years after treatment. Briefly, safety is evaluated by assessment of clinical laboratory tests, physical examination, 12-lead ECG, vital sign measurements, and by the incidence of adverse events. Efficacy may be evaluated by objective response rate (ORR) which is determined from the subjects' best response during treatment, progression free survival (PFS) which is defined as the interval from the date of randomization to the earlier of the first documentation of definitive disease progression or death from any cause, duration of response (DOR) which is defined as the interval from the date the first response (CR or PR) is achieved to the earlier of the first documentation of definitive disease progression or death from any cause, and overall survival (OS) which is defined as the interval from date of randomization to death from any cause. Pharmacokinetics is evaluated by blood samples collected at certain time points to measure AB0045 or anti-AB0045 antibodies.

The categorical and ordinal data may be summarized by count and percent of subjects, and the continuous data may be summarized by descriptive summary statistics (mean, standard deviation, minimum, quartiles, median and maximum). For the analysis of ORR, a Cochran-Mantel-Haenszel (CMH) Chi-square test on odds ratio is performed to compare the 2 treatment groups. The Kaplan-Meier (KM) method and stratified log-rank test is used to compare the two treatment groups for time-to-event endpoints (i.e, OS and PFS). A Cox proportional hazard model is used to estimate the hazard ratio and corresponding 95% confidence interval (CI). DOR is analyzed using the KM method.

### Example 10: MMP9 Inhibitor in a Refractory Model

This study used an orthotopic, syngeneic tumor model of Her2-driven breast cancer. RNA and T cell receptor (TCR) sequencing, FACS analyses, and in vitro enzymatic analyses on T cell chemoattractant CXCR3 ligands (CXCL9, CXCL10, and CXCL11) were conducted.

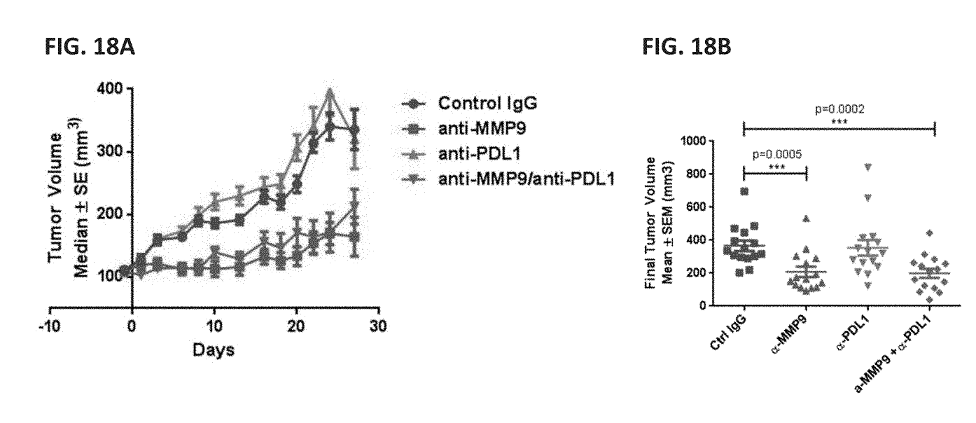

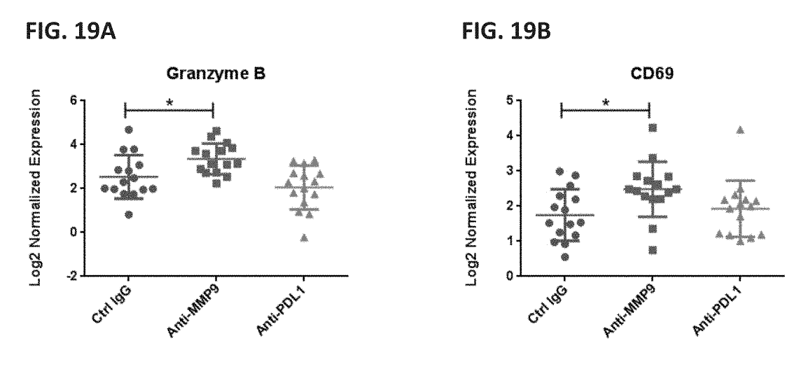

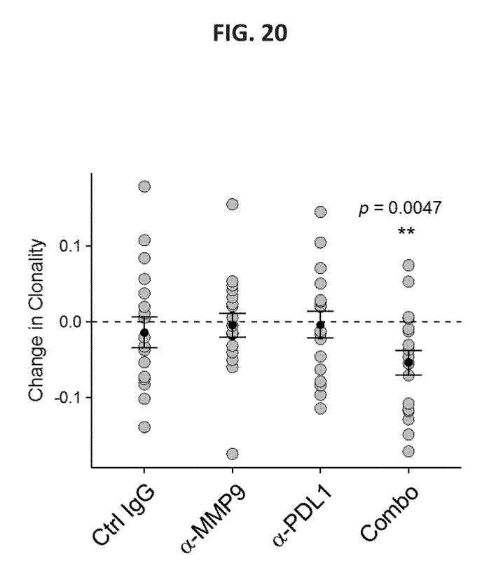

Subjects were treated with AB0046 (an anti-MMP9 monoclonal antibody which inhibited mouse MMP9 as described in WO 2013/130905) alone, anti-PD-L1 antibody (LBM1a mG1/mKap as described in US20100203056) alone, the combination of AB0046 and anti-PD-L1 antibody, or IgG (control). Results showed that the subject treated with the combination exhibited decreased primary tumor growth as compared to IgG-treated animals (p<0.01) or anti-PD-L1 alone. Data are shown in FIG. 18A-FIG. 18B. Profiling of tumors by RNA sequencing revealed that inhibition of MMP9 resulted in increased expression of genes associated with immune cell activation pathways (Hallmark Interferon Gamma Response, FDR p<0.001). Results for Granzyme B and CD69 are shown in FIG. 19A-FIG. 19B. Also, subjects treated with both anti-MMP9 and anti-PD-L1 antibodies exhibited a decrease in TCR clonality (i.e. the number of T cells with the same TCR sequence) (p=0.0047, FIG. 20). Immunophenotyping of tumor-associated T cells by flow cytometry showed that subjects treated with both anti-MMP9 and anti-PD-L1 antibodies exhibited a 2.8-fold increase in CD3+ cells in tumors (p=0.01), a 3.2-fold increase in CD4+ T cells (p=0.006), a 2.8-fold increase in CD8+ T cells (p=0.013), and a decrease in tumor-associated regulatory T cells (CD25+FoxP3+ cells, p=0.04). In vitro enzymatic analyses showed that MMP9 cleaved T cell chemoattractants and inactivated them in T cell migration assays (up to 88% reduced chemotactic activity).

### Example 11: MMP9 and Pd-L1 Inhibitors on T Cells and Effector T Cell Function in Breast Tumors

This study used orthotopic NeuT breast tumors from the mice treated with anti-MMP9 antibody (AB0046 as described in WO 2013/130905) alone, anti-PD-L1 antibody (LBM1a mG1/mKap as described in US20100203056) alone, or anti-MMP9 combined with anti-PD-L1 antibodies for phenotyping of tumor-associated T cells by polychromatic flow cytometry.

HC11-NeuT cells expressing a rat homolog of ErbB2 were generated by transduction of HC11 mammary epithelial cells with pBabe-puro NeuT retroviral construct. Puromycin-selected HC11-NeuT cells were cultured in RPMI 1640 supplemented with 8% HI-FBS, 1% GlutaMAX™, 10 ng/mL EGF, 5 μg/mL insulin and 1% penicillin-streptomycin at 5% CO₂. Early-passage HC11-NeuT cells were resuspended in serum-free medium:Matrigel™ (1:1, v/v) and 10 μL of cell suspension containing 1×10⁶ cells was inoculated into cleared mouse mammary fat pads of 3 weeks old syngeneic female Balb/c mice.

NeuT tumor growth was monitored for 3-4 weeks by palpation and treatments commenced when mean tumor volume reached 200 mm³. Each antibody (control IgG, anti-PDL1, and anti-MMP9) was administered at 20 mg/kg via i.p. injection, twice per week, in a dosing volume of 10 ml/kg. Anti-MMP9 was also administered as a single loading dose of 50 mg/kg on the morning prior to dosing start. The study was completed at 7 days after treatment initiation. Tumors were collected and examined by immunostaining and flow cytometry.

Approximately 2×10⁶ cells per sample were incubated for 30 min with rat anti-mouse CD16/CD32 monoclonal antibody (Fc Block, BD Biosciences) and subjected to immunostaining with T cell panel and Treg panel of fluorophore-conjugated monoclonal antibodies against T cell lineage markers.

For flow cytometry, side scatter and forward scatter profiles were used to eliminate debris and cell doublets, and live/dead stain was used to gate live cells, followed by gating for CD45-positive cells to select for leukocytes. Fluorescence Minus One (FMO) control was used for each fluorophore in order to identify and gate cells in the context of data spread due to polychromatic flow cytometry. Distinct T cell subsets were identified based on co-expression of multiple markers: CD3ε⁺ for CD3⁺ T cells; CD3ε⁺/CD8⁺CD4⁻ for CD8⁺ T cells; CD3ε⁺/CD8⁻CD4⁺ for CD4⁺ T cells; CD3ε⁺/CD8⁻CD4⁺/CD25⁺FoxP3⁺ for Treg cells; CD3ε⁺/CD8⁺CD4⁻/CD8⁺CD44⁺ for CD8⁺CD44⁺ cells; and CD3ε⁺/CD8⁺CD4⁻/CD4⁺CD44⁺ for CD4⁺CD44⁺ cells. Pairwise comparisons between treatment groups (Day 7) were performed using unpaired t test with Welch's correction. A p value of ≦0.05 was considered significant.

The results showed that the subjects treated with both anti-MMP9 and PD-L1 antibodies exhibited increased levels or frequencies of tumor-associated CD3, CD4, and CD8 T cells compared to those treated with either antibody alone or IgG control (Table 4). Also, the subjects treated with both anti-MMP9 and PD-L1 antibodies exhibited increased levels of CD4 and CD8 T cells with cell surface expression of CD44 (Table 5). The subject treated with MMP9 and PD-L1 inhibitors did not promote an increase in Treg (Table 4); the subject treated with anti-MMP9 antibody alone exhibited reduced level or frequency in Treg. This study suggests that the combination therapy of anti-MMP9 and anti-PD-L1 may improve T-cell mediated anti-tumor immune response.

TABLE 4
Mean Percentage ± SEM of tumor-associated T cell populations

| | | | 7 | | | |
|---|---|---|---|---|---|---|
| | | | | | | anti-PD-L1 |
| | Study day | −1 | Ctrl | anti- | anti- | and anti- |
| | Treatment | untreated | IgG | PDL1 | MMP9 | MMP9 |
| | N | 5 | 15 | 15 | 15 | 15 |
| % CD3^{+a} | Mean | 10.42 | 14.89 | 14.10 | 22.51 | 41.04 |
| | SEM | 1.13 | 5.02 | 0.95 | 6.25 | 7.34 |
| % CD8^{+a} | Mean | 2.85 | 3.36 | 3.82 | 5.26 | 9.30 |
| | SEM | 0.52 | 1.13 | 0.41 | 1.50 | 1.88 |
| % CD4^{+a} | Mean | 5.21 | 9.23 | 7.83 | 15.05 | 29.90 |
| | SEM | 0.45 | 3.93 | 0.81 | 4.83 | 5.58 |
| % Treg^{a} | Mean | 0.77 | 0.55 | 0.44 | 0.32 | 0.33 |
| | SEM | 0.10 | 0.09 | 0.07 | 0.05 | 0.10 |

^{a}% of CD45⁺ non-debris

TABLE 5
Mean Percentage ± SEM of tumor-associated CD8 and
CD4 T cells with cell surface expression of CD44

| | Study | | 7 | | | |
|---|---|---|---|---|---|---|
| | day | −1 | Ctrl | anti- | anti- | anti-PD-L1 |
| | Treat- | un- | IgG | PDL1 | MMP9 | and anti- |
| | ment | treated | | | | MMP9 |
| | N | 5 | 15 | 15 | 15 | 15 |
| % CD8⁺CD44^{+a} | Mean | 2.78 | 2.84 | 3.66 | 4.14 | 6.37 |
| | SEM | 0.51 | 0.67 | 0.42 | 0.96 | 1.17 |
| % CD4⁺CD44^{+a} | Mean | 5.15 | 8.62 | 7.54 | 13.49 | 24.53 |
| | SEM | 0.45 | 3.37 | 0.72 | 4.02 | 4.33 |

^{a}% of CD45⁺ non-debris

### Example 12: MMP9 Inhibitor in a Mouse Model of Lung Fibrosis

This study examined the effects of MMP9 and LOXL2 inhibitors in a bleomycin-induced lung fibrosis model in male C57BL/6 mice. C57BL/6 mice were treated prophylactically with anti-mMMP9 antibody (AB0046) one day prior to administration of 2 U/kg of bleomycin to induce lung fibrosis via oropharyngeal route and divided into different groups (N=5 for normal control group, N=10 for groups treated with antibodies administered intraperitoneally) as described in Table 6. Subjects in group 1 (N=5) received saline as a control to bleomycin-induced fibrosis. Subjects were administered with saline or antibodies twice a week during the study. Subjects in group 6 (normal control group which did not receive any treatment) were harvested on day 10 to determine the extent of fibrosis before treatment. The study was completed at 21 days post-bleomycin installation when fibrosis was observed in the lungs.

TABLE 6
Treatment Groups

| Group # | Bleomycin | Treatment | Dose | Schedule | N |
|---|---|---|---|---|---|
| 1 | None | Buffer | NA | 2 ×/week | 5 |
| 2 | 2 U/kg | Control IgG | 20 mg/kg | 2 ×/week | 10 |
| 3 | 2 U/kg | AB0046 | 20 mg/kg + | 2 ×/week | 10 |
| | | | 50 mg/kg loading | | |
| | | | dose | | |
| 4 | 2 U/kg | AB0023 | 15 mg/kg | 2 ×/week | 10 |
| 5 | 2 U/kg | AB0046 + | 20 mg/kg + | 2 ×/week | 10 |
| | | AB0023 | 50 mg/kg loading | | |
| | | | dose | | |
| | | | 15 mg/kg | | |
| 6 | 2 U/kg | — | — | — | 5 |

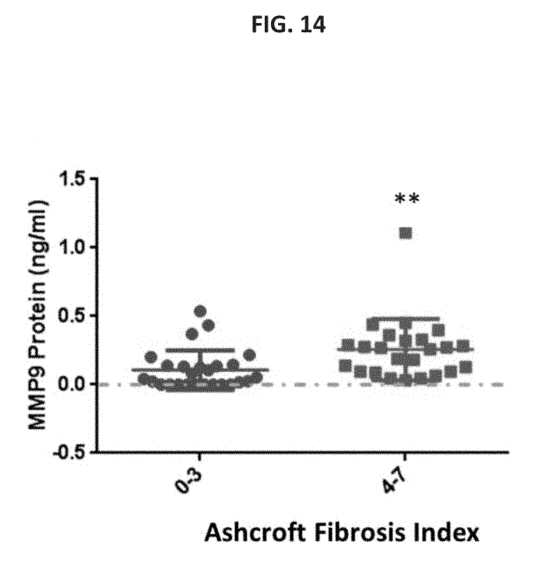

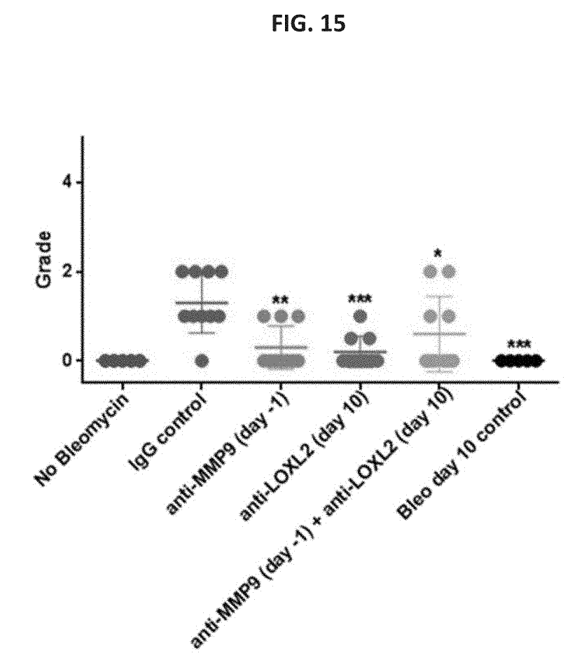

After treatments, samples were collected for leukocyte, protein, histology and weight analyses. Histopathogical staining of lung was performed by staining with Masson's trichrome and assessed for fibrosis via Ashcroft scoring. In addition, lung tissues and bronchoalveolar lavage fluid (BALf) from lung was assessed for MMP9 protein levels and activity. Leukocytes were analyzed by the Trypan Blue exclusion method and hemocytometer. MMP9 concentration was measured by ELISA. Also, the inferior lung lobe was homogenized for western blot analysis with anti-MMP9 (Abeam ab38898), anti-LOXL2 (GIL2570), α-SMA (Abeam ab5694) and anti-GAPDH (Santa Cruz Biotechnology sc-32233) antibodies. Body weight measurements over the course of the study were analyzed by ordinary one-way ANOVA with Geisser-Greenhouse correction. All groups were compared to IgG Control antibody treatment group and were found to be significantly different. Also, lung weight to body weight ratios, leukocyte counts, MMP9 protein quantification, and histopathological data were subjected to unpaired t-tests with Welch's correction. **** <0.0001; *** <0.001; ** <0.01; * <0.05. Results for these four parameters are listed in Tables 7-10 and FIG. 14-FIG. 15.

Results showed that bleomycin administration alone or following control antibody treatment resulted in decreased animal body weights, increased lung weights, increased BAL leukocyte counts, and increased MMP9 protein levels in BAL compared to normal control animals. This study indicated that prophylactic treatment of anti-MMP9 antibody may be safe and that treatment of anti-MMP9 antibody alone resulted in reduced animal lung weights with a concomitant decrease in fibrosis.

TABLE 7
Lung Weight to Body Weight Ratio

| Treatment Group | Average | Standard Deviation | p-Value |
|---|---|---|---|
| 1 | 0.968 | 0.0377 | <0.0001 |
| 2 | 1.886 | 0.2814 | — |
| 3 | 1.535 | 0.2880 | 0.0130 |
| 4 | 1.305 | 0.1859 | 0.0065 |
| 5 | 1.442 | 0.2989 | 0.0463 |

TABLE 8
BALf Leukocyte Counts

| Treatment Group | Average | Standard Deviation | p-Value |
|---|---|---|---|
| 1 | 54250 | 27166 | 0.0005 |
| 2 | 226500 | 106899 | — |
| 3 | 243625 | 97251 | ns |
| 4 | 192375 | 100228 | ns |
| 5 | 167500 | 167500 | ns |

ns: not significant

TABLE 9
Ashcroft Scoring for Fibrosis Assessment

| Treatment Group | Average | Standard Deviation | p-Value |
|---|---|---|---|
| 1 | 0 | 0 | <0.0001 |
| 2 | 4.170 | 1.372 | — |
| 3 | 2.640 | 1.626 | 0.0358 |
| 4 | 2.470 | 1.725 | 0.0259 |
| 5 | 3.140 | 1.874 | ns |

ns: not significant

TABLE 10
MMP9 BALf Protein Levels

| Treatment Group | Average | Standard Deviation | p-Value |
|---|---|---|---|
| 1 | −0.0461 | 0.04056 | 0.0131 |
| 2 | 0.2643 | 0.3177 | — |
| 3 | 0.2281 | 0.1889 | ns |
| 4 | 0.17898 | 0.1602 | ns |
| 5 | 0.0875 | 0.1148 | ns |

ns: not significant

All mice treated with bleomycin lost weight as compared to saline-treated control mice (data not shown). However, mice treated with anti-MMP9 antibody, singly or in combination with anti-LOXL2 antibody, showed reduced body weight loss as compared to the IgG control antibody-treated group (bleomycin control arm not included in statistical analyses) (p=0.0130). Anti-LOXL2 antibody treated alone also resulted in a significant reduction in body weight loss (p=0.065).

At the end of the study, mouse lungs were dissected and weighed. Bleomycin instillation resulted in increased lung weight to body weight ratios in all bleomycin-treated groups as compared to saline-treated controls, consistent with increased lung fibrosis. Mice treated with the anti-MMP9 antibody, singly or in combination with anti-LOXL2 antibody dosed therapeutically on day 10, had decreased lung weight to body weight ratios as compared to the IgG control antibody treated group (p=0.013 and p=0.0463, respectively) (Table 7).

Leukocyte counts were increased in the bleomycin-administered mice. However, no significant differences were observed between the control IgG and the anti-MMP9 or anti-LOXL2 antibody treated mice (Table 8). This suggests that anti-MMP9 antibody treatment did not have any anti- or pro-inflammatory effects.

As anti-MMP9 antibody treatment appeared to show benefit to bleomycin-treated animals as determined by the reduction observed in final lung weights, the degree of fibrosis present in the treated animals' lungs was assessed next (Table 9). As shown in Table 10, treatment with anti-MMP9 antibody did not result in decreased MMP9 total protein levels. Additionally, results suggested that total MMP9 levels may be associated with disease severity (p=0.008) (FIG. 14).

### Example 13: MMP9 Inhibitor in the Presence of Human Neutrophil Elastase and Cystic Fibrosis Sputa

Proteolyzed antibodies, likely cleaved at the hinge region, were observed in CF patient sputum (Sloane, A. J. et al. Proteomic analysis of sputum from adults and children with cystic fibrosis and from control subjects. Am J Respir Crit Care Med (2005) 172: 1416-1426). It was hypothesized that human neutrophil elastase (HNE), which is elevated in the CF airway, and other proteases may mediate antibody proteolysis. This in vitro study characterized the stability of anti-MMP9 antibody AB0045 (an IgG4 antibody that binds and inhibits MMP9 independent of the Fc region of the antibody) in presence of HNE or sputum from CF subjects.

AB0045 was incubated with recombinant HNE (Enzo Biosciences (BML-SE284) or sputa from two distinct CF subjects at 37° C. for 24 hours. AB0045 was also digested to completion at the hinge region with FabRicator™ enzyme. Protein degradation was monitored via Coomassie blue staining of non-reducing SDS-PAGE gels. Binding affinity to MMP9 was measured by surface plasmon resonance, and inhibition of MMP9 proteolysis was determined by a fluorescently labeled MMP9 substrate peptide (ES001, R&D systems). AB0045 bound MMP9 was measured by a modified ELISA from R&D systems (DMP 900). In addition, total MMP9 and free MMP9 (MMP9 not bound to AB0045) was measured, and bound MMP9 was determined as the difference between total MMP9 and free MMP9.

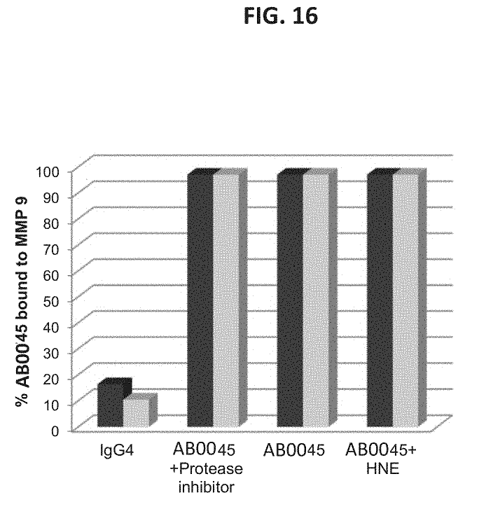

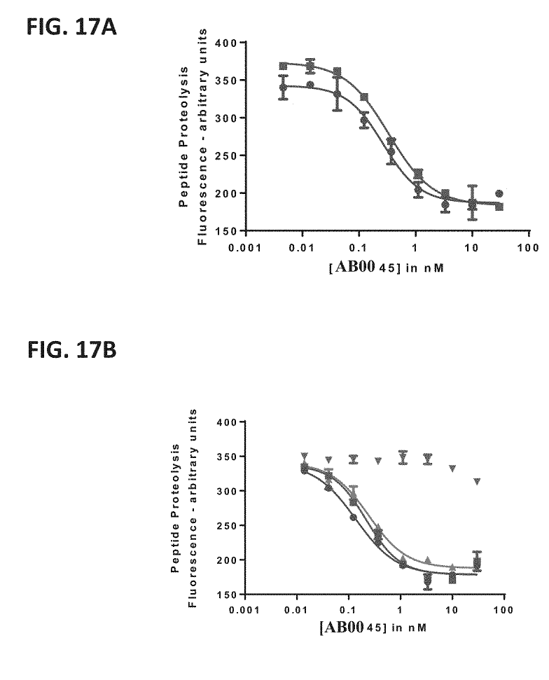

Results of protein degradation analysis showed that <20% of AB0045 was proteolyzed after incubation with HNE or CF sputa (data not shown). The proteolysis products were consistent with cleavage at the hinge region (data not shown). Complete digestion of the AB0045 at the hinge did not reduce binding to MMP9. Also, results showed that CF sputum or spiked HNE did not reduce or affect the binding of AB0045 to MMP9 (FIG. 16) and that AB0045 inhibited MMP9 activity in presence of exogenous HNE and CF sputum (FIG. 17A-FIG. 17B).

All of the above U.S. patents, U.S. patent application publications, U.S. patent applications, foreign patents, foreign patent applications and non-patent publications referred to in this specification and/or listed in the Application Data Sheet are incorporated herein by reference in their entirety.

From the foregoing it will be appreciated that, although specific embodiments of the invention have been described herein for purposes of illustration, various modifications may be made without deviating from the spirit and scope of the present application.

What is claimed is:

2. A method of treating or preventing a disease or condition in a subject in need thereof, comprising providing to the subject:
(i) an effective amount of an anti-Matrix Metalloproteinase 9 (MMP9) antibody or antigen binding fragment thereof; and
(ii) optionally, an effective amount of one or more additional therapeutic agent, thereby treating or preventing the disease or condition in the subject.

3. The method of claim 1, wherein the anti-MMP9 antibody or antigen binding fragment thereof binds to an epitope of MMP9, wherein the epitope comprises amino acid residues 104-119, residues 159-166, or residues 191-202 of SEQ ID NO: 27.

4. The method of claim 1, wherein the anti-MMP9 antibody or antigen binding fragment thereof comprises a heavy chain variable (VH) region comprising a complementarity determining region (CDR) having an amino acid sequence selected from the group consisting of SEQ ID NOs: 13, 14 and 15 and/or a light chain variable (VL) region having a complementarity determining region (CDR) having an amino acid sequence selected from the group consisting of SEQ ID NOs: 16, 17 and 18.

5. The method of claim 1, wherein the anti-MMP9 antibody or antigen binding fragment thereof comprises a VH region comprising an amino acid sequence selected from the group consisting of SEQ ID NOs: 3, 5, 6, 7 and 8 and/or a VL region having an amino acid sequence selected from the group consisting of SEQ ID NOs: 4, 9, 10, 11 and 12.

6. The method of claim 1, wherein the anti-MMP9 antibody or antigen binding fragment thereof is humanized, chimeric or human.

7. The method of claim 1, wherein the anti-MMP9 antibody or antigen binding fragment thereof inhibits the enzymatic activity of MMP9.

8. The method of claim 1, wherein the disease or condition is cystic fibrosis; a cancer; an autoimmune or inflammatory disease or condition; vasculitis; septicemia; multiple sclerosis, muscular dystrophy; lupus; allergy; or asthma.

9. The method of claim 1, wherein the disease or condition is myeloid cell-associated inflammation, cystic fibrosis; non-cystic fibrosis bronchiectasis, sarcoidosis, idiopathic pulmonary fibrosis, tuberculosis, breast cancer, pancreatic cancer, esophagogastric adenocarcinoma, non-small cell lung cancer, lung squamous cell carcinoma, lung adenocarcinoma, gastric adenocarcinoma, colorectal carcinoma, pancreatic adenocarcinoma, head and neck squamous cell carcinoma, hepatocellular carcinoma, colorectal cancer, colorectal adenocarcinoma, hepatocellular carcinoma, rheumatoid arthritis, an inflammatory bowel disease (IBD), ulcerative colitis (UC), Crohn's disease (CD), indeterminate colitis; large vessel vasculitis, Takayasu arteritis and Giant cell arteritis, medium vessel vasculitis, Polyarteritis Nodosa, Kawasaki Disease, immune complex small vessel vasculitis, Cryoglobulinemic vasculitis, IgA vasculitis (Henoch-Schonlein), hypocomplementemic urticarial vasculitis (anti-C1q vasculitis), anti-GBM Disease, ANCA-associated small vessel vasculitis, microscopic polyangiitis, granulomatosis with polyangiitis (Wegner's), and eosinophilic granulomatosis with polyangiitis (Churg-Strauss), septicemia, multiple sclerosis, muscular dystrophy, lupus, allergy, asthma, or hidradenitis suppurativa.

10. The method of claim 1, wherein the anti-MMP9 antibody or antigen binding fragment thereof is administered concurrently or sequentially with the additional therapeutic agent.

11. The method of claim 1, the anti-MMP9 antibody or antigen binding fragment thereof and the additional therapeutic agent are administered in one pharmaceutical composition.

12. The method of claim 1, wherein the anti-MMP9 antibody or antigen binding fragment thereof is administered at a dose of about 100 mg, of about 150 mg, of about 200 mg, of about 300 mg, or of about 400 mg.

13. The method of claim 1, the anti-MMP9 antibody or antigen binding fragment thereof is administered once every week, once every two weeks, or once every three weeks.

14. The method of claim 1, the anti-MMP9 antibody or antigen binding fragment thereof and/or the additional therapeutic agent is administered intravenously, intradermally, or subcutaneously.

15. The method of claim 1, wherein the additional therapeutic agent is chemotherapeutic agent, an anti-angiogenic agent, an anti-fibrotic agent, an anti-inflammatory agent, an immune modulating agent, an immunotherapeutic agent, a therapeutic antibody, a radiotherapeutic agent, an anti-neoplastic agent or an anti-cancer agent, an anti-proliferation agent, or any combination thereof.

16. The method of claim 1, wherein the additional therapeutic agent is the immune modulating agent is anti-CTLA-4 antibody, anti-LAG-3 antibody, anti-B7-H3 antibody, anti-B7-H4 antibody, anti-Tim3 antibody, anti-BTLA antibody, anti-KIR antibody, anti-A2aR antibody, anti CD200 antibody, anti-PD-1 antibody, anti-PD-L1 antibody, anti-CD28 antibody, anti-CD80 or -CD86 antibody, anti-B7RP1 antibody, anti-B7-H3 antibody, anti-HVEM antibody, anti-CD137 or -CD137L antibody, anti-OX40 or -OX40L antibody, anti-CD40 or -CD40L antibody, anti-GALS antibody, anti-IL-10 antibody or A2aR drug.

17. A pharmaceutical composition comprising:
a) a pharmaceutically acceptable excipient,
b) an anti-MMP9 antibody or antigen binding fragment thereof; and
c) an additional therapeutic agent.

18. The pharmaceutical composition of claim 16, wherein the anti-MMP9 antibody or antigen binding fragment thereof binds to an epitope of MMP9, wherein the epitope comprises amino acid residues 104-119, residues 159-166, or residues 191-202 of SEQ ID NO: 27.

19. The pharmaceutical composition of claim 16, wherein the anti-MMP9 antibody or antigen binding fragment thereof comprises a heavy chain variable (VH) region comprising a complementarity determining region (CDR) having an amino acid sequence selected from the group consisting of SEQ ID NOs: 13, 14 and 15 and/or a light chain variable (VL) region having a complementarity determining region (CDR) having an amino acid sequence selected from the group consisting of SEQ ID NOs: 16, 17 and 18.

20. The pharmaceutical composition of claim 16, wherein the anti-MMP9 antibody or antigen binding fragment thereof comprises a VH region comprising an amino acid sequence selected from the group consisting of SEQ ID NOs: 3, 5, 6, 7 and 8 and/or a VL region having an amino acid sequence selected from the group consisting of SEQ ID NOs: 4, 9, 10, 11 and 12.

21. The pharmaceutical composition of claim 16, the composition is administered once every week, once every two weeks, or once every three weeks.

22. The pharmaceutical composition of claim 16, the composition is administered intravenously, intradermally, or subcutaneously.

23. The pharmaceutical composition of claim 16, wherein the additional therapeutic agent is chemotherapeutic agent, an anti-angiogenic agent, an anti-fibrotic agent, an anti-inflammatory agent, an immune modulating agent, an immunotherapeutic agent, a therapeutic antibody, a radiotherapeutic agent, an anti-neoplastic agent or an anti-cancer agent, an anti-proliferation agent, or any combination thereof.

24. The pharmaceutical composition of claim 16, wherein the additional therapeutic agent is the immune modulating agent is anti-CTLA-4 antibody, anti-LAG-3 antibody, anti-B7-H3 antibody, anti-B7-H4 antibody, anti-Tim3 antibody, anti-BTLA antibody, anti-KIR antibody, anti-A2aR antibody, anti CD200 antibody, anti-PD-1 antibody, anti-PD-L1 antibody, anti-CD28 antibody, anti-CD80 or -CD86 antibody, anti-B7RP1 antibody, anti-B7-H3 antibody, anti-HVEM antibody, anti-CD137 or -CD137L antibody, anti-OX40 or -OX40L antibody, anti-CD40 or -CD40L antibody, anti-GALS antibody, anti-IL-10 antibody or A2aR drug.

25. A kit for treating or preventing a disease or condition in a subject in need thereof, comprising:
a) an anti-MMP9 antibody or antigen binding fragment thereof; and
b) an additional therapeutic agent.


SEQUENCE LISTING (60 sequences)

SEQ ID NO: 1 - protein, 470 aa, Mus musculus
  CHAIN (1)..(470): AB0041 heavy chain
  PEPTIDE (1)..(19): signal peptide
  misc_feature (135)..(470): IgG2b constant region
MAVLVLFLCLVAFPSCVLSQVQLKESGPGLVAPSQSLSITCTVSGFSLLSYGVHWVRQPPGKGLEWLGVIWTGGTTNYNSALMSRLSISKDDSKSQVFLKMNSLQTDDTAIYYCARYYYGMDYWGQGTSVTVSSAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVRISWFVNNVEVHTAQTQTHREDYNSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRSPGK

SEQ ID NO: 2 - protein, 234 aa, Mus musculus
  CHAIN (1)..(234): AB0041 light chain
  PEPTIDE (1)..(20): signal peptide
  misc_feature (128)..(234): kappa constant region
MESQIQVFVFVFLWLSGVDGDIVMTQSHKFMSTSVGDRVSITCKASQDVRNTVAWYQQKTGQSPKLLIYSSSYRNTGVPDRFTGSGSGTDFTFTISSVQAEDLAVYFCQQHYITPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 3 - protein, 115 aa, Mus musculus
  CHAIN (1)..(115): variable region of the IgG2b heavy chain of AB0041
  misc_feature (26)..(35): complementarity-determining region (CDR)
  misc_feature (50)..(65): complementarity-determining region (CDR)
  misc_feature (98)..(104): complementarity-determining region (CDR)
QVQLKESGPGLVAPSQSLSITCTVSGFSLLSYGVHWVRQPPGKGLEWLGVIWTGGTTNYNSALMSRLSISKDDSKSQVFLKMNSLQTDDTAIYYCARYYYGMDYWGQGTSVTVSS

SEQ ID NO: 4 - protein, 107 aa, Mus musculus
  CHAIN (1)..(107): variable region of the kappa light chain of AB0041
  misc_feature (24)..(34): complementarity-determining region (CDR)
  misc_feature (50)..(56): complementarity-determining region (CDR)
  misc_feature (89)..(97): complementarity-determining region (CDR)
DIVMTQSHKFMSTSVGDRVSITCKASQDVRNTVAWYQQKTGQSPKLLIYSSSYRNTGVPDRFTGSGSGTDFTFTISSVQAEDLAVYFCQQHYITPYTFGGGTKLEIK

SEQ ID NO: 5 - protein, 115 aa, Artificial Sequence
  synthetic construct
  VARIANT (1)..(115): VH1 heavy chain variant
QVQLQESGPGLVKPSETLSLTCTVSGFSLLSYGVHWVRQPPGKGLEWLGVIWTGGTTNYNSALMSRLTISKDDSKSTVYLKMNSLKTEDTAIYYCARYYYGMDYWGQGTSVTVSS

SEQ ID NO: 6 - protein, 115 aa, Artificial Sequence
  synthetic construct
  VARIANT (1)..(115): VH2 heavy chain variant
QVQLQESGPGLVKPSETLSLTCTVSGFSLLSYGVHWVRQPPGKGLEWLGVIWTGGTTNYNSALMSRLTISKDDSKNTVYLKMNSLKTEDTAIYYCARYYYGMDYWGQGTLVTVSS

SEQ ID NO: 7 - protein, 115 aa, Artificial Sequence
  synthetic construct
  VARIANT (1)..(115): VH3 heavy chain variant
QVQLQESGPGLVKPSETLSLTCTVSGFSLLSYGVHWVRQPPGKGLEWLGVIWTGGTTNYNSALMSRFTISKDDSKNTVYLKMNSLKTEDTAIYYCARYYYGMDYWGQGTLVTVSS

SEQ ID NO: 8 - protein, 115 aa, Artificial Sequence
  synthetic construct
  VARIANT (1)..(115): VH4 heavy chain variant
QVQLQESGPGLVKPSETLSLTCTVSGFSLLSYGVHWVRQPPGKGLEWLGVIWTGGTTNYNSALMSRFTISKDDSKNTLYLKMNSLKTEDTAIYYCARYYYGMDYWGQGTLVTVSS

SEQ ID NO: 9 - protein, 107 aa, Artificial Sequence
  synthetic construct
  VARIANT (1)..(107): Vk1 light chain variant
DIVMTQSPSFLSASVGDRVTITCKASQDVRNTVAWYQQKTGKAPKLLIYSSSYRNTGVPDRFTGSGSGTDFTLTISSLQAEDVAVYFCQQHYITPYTFGGGTKVEIK

SEQ ID NO: 10 - protein, 107 aa, Artificial Sequence
  synthetic construct
  VARIANT (1)..(107): Vk2 light chain variant
DIVMTQSPSSLSASVGDRVTITCKASQDVRNTVAWYQQKPGKAPKLLIYSSSYRNTGVPDRFTGSGSGTDFTLTISSLQAEDVAVYFCQQHYITPYTFGGGTKVEIK

SEQ ID NO: 11 - protein, 107 aa, Artificial Sequence
  synthetic construct
  VARIANT (1)..(107): Vk3 light chain variant
DIQMTQSPSSLSASVGDRVTITCKASQDVRNTVAWYQQKPGKAPKLLIYSSSYRNTGVPDRFSGSGSGTDFTLTISSLQAEDVAVYFCQQHYITPYTFGGGTKVEIK

SEQ ID NO: 12 - protein, 107 aa, Artificial Sequence
  synthetic construct
  VARIANT (1)..(107): Vk4 light chain variant
DIQMTQSPSSLSASVGDRVTITCKASQDVRNTVAWYQQKPGKAPKLLIYSSSYRNTGVPDRFSGSGSGTDFTLTISSLQAEDVAVYYCQQHYITPYTFGGGTKVEIK

SEQ ID NO: 13 - protein, 10 aa, Artificial Sequence
  synthetic construct
  misc_feature (1)..(10): complementarity-determining region (CDR1) of heavy chain of anti-MMP9 antibody
GFSLLSYGVH

SEQ ID NO: 14 - protein, 16 aa, Artificial Sequence
  synthetic construct
  misc_feature (1)..(16): complementarity-determining region (CDR2) of heavy chain of anti-MMP9 antibody
VIWTGGTTNYNSALMS

SEQ ID NO: 15 - protein, 7 aa, Artificial Sequence
  synthetic construct
  misc_feature (1)..(7): complementarity-determining region (CDR3) of heavy chain of anti-MMP9 antibody
YYYGMDY

SEQ ID NO: 16 - protein, 11 aa, Artificial Sequence
  synthetic construct
  misc_feature (1)..(11): complementarity-determining region (CDR1) of light chain of anti-MMP9 antibody
KASQDVRNTVA

SEQ ID NO: 17 - protein, 7 aa, Artificial Sequence
  synthetic construct
  misc_feature (1)..(7): complementarity-determining region (CDR2) of light chain of anti-MMP9 antibody
SSSYRNT

SEQ ID NO: 18 - protein, 9 aa, Artificial Sequence
  synthetic construct
  misc_feature (1)..(9): complementarity-determining region (CDR3) of light chain of anti-MMP9 antibody
QQHYITPYT

SEQ ID NO: 19 - DNA, 345 bp, Artificial Sequence
  synthetic construct
  misc_feature (1)..(345): nucleotide sequence encoding VH1 heavy chain amino acid sequence
caggtgcagctgcaggaatccggccctggcctggtcaagccctccgagacactgtccctgacctgcaccgtgtccggcttctccctgctgtcctacggcgtgcactgggtccgacagcctccagggaagggcctggaatggctgggcgtgatctggaccggcggcaccaccaactacaactccgccctgatgtcccggctgaccatctccaaggacgactccaagtccaccgtgtacctgaagatgaactccctgaaaaccgaggacaccgccatctactactgcgcccggtactactacggcatggactactggggccagggcacctccgtgaccgtgtcctca

SEQ ID NO: 20 - DNA, 345 bp, Artificial Sequence
  synthetic construct
  misc_feature (1)..(345): nucleotide sequence encoding VH2 heavy chain amino acid sequence
caggtgcagctgcaggaatccggccctggcctggtcaagccctccgagacactgtccctgacctgcaccgtgtccggcttctccctgctgtcctacggcgtgcactgggtccgacagcctccaggcaaaggcctggaatggctgggcgtgatctggaccggcggcaccaccaactacaactccgccctgatgtcccggctgaccatctccaaggacgactccaagaacaccgtgtacctgaagatgaactccctgaaaaccgaggacaccgccatctactactgcgcccggtactactacggcatggactactggggccagggcaccctggtcaccgtgtcctca

SEQ ID NO: 21 - DNA, 345 bp, Artificial Sequence
  synthetic construct
  misc_feature (1)..(345): nucleotide sequence encoding VH3 heavy chain amino acid sequence
caggtgcagctgcaggaatccggccctggcctggtcaagccctccgagacactgtccctgacctgcaccgtgtccggcttctccctgctgtcctacggcgtgcactgggtccgacagcctccaggcaaaggcctggaatggctgggcgtgatctggaccggcggcaccaccaactacaactccgccctgatgtcccggttcaccatctccaaggacgactccaagaacaccgtgtacctgaagatgaactccctgaaaaccgaggacaccgccatctactactgcgcccggtactactacggcatggactactggggccagggcaccctggtcaccgtgtcctca

SEQ ID NO: 22 - DNA, 345 bp, Artificial Sequence
  synthetic construct
  misc_feature (1)..(345): nucleotide sequence encoding VH4 heavy chain amino acid sequence
caggtgcagctgcaggaatccggccctggcctggtcaagccctccgagacactgtccctgacctgcaccgtgtccggcttctccctgctgtcctacggcgtgcactgggtccgacagcctccaggcaaaggcctggaatggctgggcgtgatctggaccggcggcaccaccaactacaactccgccctgatgtcccggttcaccatctccaaggacgactccaagaacaccctgtacctgaagatgaactccctgaaaaccgaggacaccgccatctactactgcgcccggtactactacggcatggactactggggccagggcaccctggtcaccgtgtcctca

SEQ ID NO: 23 - DNA, 321 bp, Artificial Sequence
  synthetic construct
  misc_feature (1)..(321): nucleotide sequence encoding Vk1 light chain amino acid sequence
gacatcgtgatgacccagtcccccagcttcctgtccgcctccgtgggcgacagagtgaccatcacatgcaaggcctctcaggacgtgcggaacaccgtggcctggtatcagcagaaaaccggcaaggcccccaagctgctgatctactcctcctcctaccggaacaccggcgtgcccgaccggtttaccggctctggctccggcaccgactttaccctgaccatcagctccctgcaggccgaggacgtggccgtgtacttctgccagcagcactacatcaccccctacaccttcggcggaggcaccaaggtggaaataaaa

SEQ ID NO: 24 - DNA, 321 bp, Artificial Sequence
  synthetic construct
  misc_feature (1)..(321): nucleotide sequence encoding Vk2 light chain amino acid sequence
gacatcgtgatgacccagtccccctccagcctgtccgcctctgtgggcgacagagtgaccatcacatgcaaggcctctcaggacgtgcggaacaccgtggcctggtatcagcagaagcccggcaaggcccccaagctgctgatctactcctcctcctaccggaacaccggcgtgcccgaccggtttaccggctctggctccggcaccgactttaccctgaccatcagctccctgcaggccgaggacgtggccgtgtacttctgccagcagcactacatcaccccctacaccttcggcggaggcaccaaggtggaaataaaa

SEQ ID NO: 25 - DNA, 321 bp, Artificial Sequence
  synthetic construct
  misc_feature (1)..(321): nucleotide sequence encoding Vk3 light chain amino acid sequence
gacatccagatgacccagtccccctccagcctgtccgcctctgtgggcgacagagtgaccatcacatgcaaggcctcccaggacgtgcggaacaccgtggcctggtatcagcagaagcccggcaaggcccccaagctgctgatctactcctcctcctaccggaacaccggcgtgcccgaccggttctctggctctggaagcggcaccgactttaccctgaccatcagctccctgcaggccgaggacgtggccgtgtacttctgccagcagcactacatcaccccctacaccttcggcggaggcaccaaggtggaaataaaa

SEQ ID NO: 26 - DNA, 321 bp, Artificial Sequence
  synthetic construct
  misc_feature (1)..(321): nucleotide sequence encoding Vk4 light chain amino acid sequence
gacatccagatgacccagtccccctccagcctgtccgcctctgtgggcgacagagtgaccatcacatgcaaggcctctcaggacgtgcggaacaccgtggcctggtatcagcagaagcccggcaaggcccccaagctgctgatctactcctcctcctaccggaacaccggcgtgcccgaccggttctctggctctggaagcggcaccgactttaccctgaccatcagctccctgcaggccgaggacgtggccgtgtactactgccagcagcactacatcaccccctacaccttcggcggaggcaccaaggtggaaataaaa

SEQ ID NO: 27 - protein, 707 aa, Homo sapiens
  misc_feature (1)..(707): matrix metalloproteinase 9 (MMP9)
  PEPTIDE (1)..(19): signal peptide
  DOMAIN (38)..(98): peptidoglycan binding domain
  SITE (98)..(99): propeptide cleavage site
  DOMAIN (112)..(445): Zn dependent metalloproteinase domain
  DOMAIN (223)..(271): fibronectin type II domain (gelatin binding domain)
  DOMAIN (281)..(329): fibronectin type II domain (gelatin binding domain)
  DOMAIN (340)..(388): fibronectin type II domain (gelatin binding domain)
  misc_feature (400)..(411): Zn binding region
  DOMAIN (521)..(565): hemopexin-like domain
  DOMAIN (567)..(608): hemopexin-like domain
  DOMAIN (613)..(659): hemopexin-like domain
  DOMAIN (661)..(704): hemopexin-like domain
MSLWQPLVLVLLVLGCCFAAPRQRQSTLVLFPGDLRTNLTDRQLAEEYLYRYGYTRVAEMRGESKSLGPALLLLQKQLSLPETGELDSATLKAMRTPRCGVPDLGRFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGVVVPTRFGNADGAACHFPFIFEGRSYSACTTDGRSDGLPWCSTTANYDTDDRFGFCPSERLYTRDGNADGKPCQFPFIFQGQSYSACTTDGRSDGYRWCATTANYDRDKLFGFCPTRADSTVMGGNSAGELCVFPFTFLGKEYSTCTSEGRGDGRLWCATTSNFDSDKKWGFCPDQGYSLFLVAAHEFGHALGLDHSSVPEALMYPMYRFTEGPPLHKDDVNGIRHLYGPRPEPEPRPPTTTTPQPTAPPTVCPTGPPTVHPSERPTAGPTGPPSAGPTGPPTAGPSTATTVPLSPVDDACNVNIFDAIAEIGNQLYLFKDGKYWRFSEGRGSRPQGPFLIADKWPALPRKLDSVFEEPLSKKLFFFSGRQVWVYTGASVLGPRRLDKLGLGADVAQVTGALRSGRGKMLLFSGRRLWRFDVKAQMVDPRSASEVDRMFPGVPLDTHDVFQYREKAYFCQDRFYWRVSSRSELNQVDQVGYVTYDILQCPED

SEQ ID NO: 28 - protein, 688 aa, Homo sapiens
  misc_feature (1)..(688): mature full-length matrix metalloproteinase 9 (MMP9)
APRQRQSTLVLFPGDLRTNLTDRQLAEEYLYRYGYTRVAEMRGESKSLGPALLLLQKQLSLPETGELDSATLKAMRTPRCGVPDLGRFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGVVVPTRFGNADGAACHFPFIFEGRSYSACTTDGRSDGLPWCSTTANYDTDDRFGFCPSERLYTRDGNADGKPCQFPFIFQGQSYSACTTDGRSDGYRWCATTANYDRDKLFGFCPTRADSTVMGGNSAGELCVFPFTFLGKEYSTCTSEGRGDGRLWCATTSNFDSDKKWGFCPDQGYSLFLVAAHEFGHALGLDHSSVPEALMYPMYRFTEGPPLHKDDVNGIRHLYGPRPEPEPRPPTTTTPQPTAPPTVCPTGPPTVHPSERPTAGPTGPPSAGPTGPPTAGPSTATTVPLSPVDDACNVNIFDAIAEIGNQLYLFKDGKYWRFSEGRGSRPQGPFLIADKWPALPRKLDSVFEEPLSKKLFFFSGRQVWVYTGASVLGPRRLDKLGLGADVAQVTGALRSGRGKMLLFSGRRLWRFDVKAQMVDPRSASEVDRMFPGVPLDTHDVFQYREKAYFCQDRFYWRVSSRSELNQVDQVGYVTYDILQCPED

SEQ ID NO: 29 - protein, 19 aa, Homo sapiens
MSLWQPLVLVLLVLGCCFA

SEQ ID NO: 30 - protein, 470 aa, Mus musculus
  CHAIN (1)..(470): M4 heavy chain (IgG2b)
MAVLVLFLCLVAFPSCVLSQVQLKESGPGLVAPSQSLSITCTVSGFSLLSYGVHWVRQPPGKGLEWLGVIWTGGSTNYNSALMSRLSISKDDSKSQVFLKMNSLQTDDTAMYYCARYYYAMDYWGQGTSVTVSSAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVRISWFVNNVEVHTAQTQTHREDYNSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRSPGK

SEQ ID NO: 31 - protein, 234 aa, Mus musculus
  CHAIN (1)..(234): M4 light chain (kappa)
MESQIQVFVFVFLWLSGVDGDIVMTQSHKFMFTSVGDRVSITCKASQDVRNTVAWYQQKTGQSPKLLIYSASYRNTGVPDRFTGSISGTDFTFTISSVQAEDLALYYCQQHYSTPYTFGGGTKLEVKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 32 - protein, 115 aa, Mus musculus
QVQLKESGPGLVAPSQSLSITCTVSGFSLLSYGVHWVRQPPGKGLEWLGVIWTGGSTNYNSALMSRLSISKDDSKSQVFLKMNSLQTDDTAMYYCARYYYAMDYWGQGTSVTVSS

SEQ ID NO: 33 - protein, 107 aa, Mus musculus
DIVMTQSHKFMFTSVGDRVSITCKASQDVRNTVAWYQQKTGQSPKLLIYSASYRNTGVPDRFTGSISGTDFTFTISSVQAEDLALYYCQQHYSTPYTFGGGTKLEVK

SEQ ID NO: 34 - protein, 10 aa, Mus musculus
GFSLLSYGVH

SEQ ID NO: 35 - protein, 16 aa, Mus musculus
VIWTGGSTNYNSALMS

SEQ ID NO: 36 - protein, 7 aa, Mus musculus
YYYAMDY

SEQ ID NO: 37 - protein, 11 aa, Mus musculus
KASQDVRNTVA

SEQ ID NO: 38 - protein, 7 aa, Mus musculus
SASYRNT

SEQ ID NO: 39 - protein, 9 aa, Mus musculus
QQHYSTPYT

SEQ ID NO: 40 - protein, 229 aa, Mus musculus
  CHAIN (1)..(229): M12 kappa chain
QVFVYMLLWLSGVDGDIVMTQSQKFMSTSVGDRVSVTCKASQNVGTNVAWYQQKPGQSPKALIYSASYRFSGVPDRFTGSGSGTDFTLTISNVQSEDLAEYFCQQYNSYPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 41 - protein, 107 aa, Mus musculus
DIVMTQSQKFMSTSVGDRVSVTCKASQNVGTNVAWYQQKPGQSPKALIYSASYRFSGVPDRFTGSGSGTDFTLTISNVQSEDLAEYFCQQYNSYPYTFGGGTKLEIK

SEQ ID NO: 42 - protein, 11 aa, Mus musculus
KASQNVGTNVA

SEQ ID NO: 43 - protein, 7 aa, Mus musculus
SASYRFS

SEQ ID NO: 44 - protein, 9 aa, Mus musculus
QQYNSYPYT

SEQ ID NO: 45 - protein, 233 aa, Mus musculus
  CHAIN (1)..(233): AB0046 kappa light chain
MSSAQFLGLLLLCFQGTRCDIQMTQTTSSLSASLGDRVTISCSASQGISNYLNWYQQKPDGTFKLLIYYTSILHSGVPSRFSGSGSGTDYSLTISNLEPEDIATYYCQQYGWLPRTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 46 - protein, 460 aa, Mus musculus
  CHAIN (1)..(460): AB0046 IgG1 heavy chain
MGWSSIILFLVATATGVHSQVQLQQPGSVLVRPGASVKLSCTASGYTFTSYWMNWVKQRPGQGLEWIGEIYPISGRTNYNEKFKVKATLTVDTSSSTAYMDLNSLTSEDSAVYYCARSRANWDDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 47 - protein, 117 aa, Mus musculus
QVQLQQPGSVLVRPGASVKLSCTASGYTFTSYWMNWVKQRPGQGLEWIGEIYPISGRTNYNEKFKVKATLTVDTSSSTAYMDLNSLTSEDSAVYYCARSRANWDDYWGQGTTLTVSS

SEQ ID NO: 48 - protein, 107 aa, Mus musculus
DIQMTQTTSSLSASLGDRVTISCSASQGISNYLNWYQQKPDGTFKLLIYYTSILHSGVPSRFSGSGSGTDYSLTISNLEPEDIATYYCQQYGWLPRTFGGGTKLEIK

SEQ ID NO: 49 - protein, 461 aa, Artificial Sequence
  synthetic construct
  CHAIN (1)..(461): AB0045 heavy chain
MGWSLILLFLVAVATRVHSQVQLQESGPGLVKPSETLSLTCTVSGFSLLSYGVHWVRQPPGKGLEWLGVIWTGGTTNYNSALMSRFTISKDDSKNTVYLKMNSLKTEDTAIYYCARYYYGMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 50 - protein, 234 aa, Artificial Sequence
  synthetic construct
  CHAIN (1)..(234): AB0045 light chain
MRVPAQLLGLLLLWLPGARCDIQMTQSPSSLSASVGDRVTITCKASQDVRNTVAWYQQKPGKAPKLLIYSSSYRNTGVPDRFSGSGSGTDFTLTISSLQAEDVAVYYCQQHYITPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 148 aa, Mus musculus
MESQIQVFVFVFLWLSGVDGDIVMTQSHKFMFTSVGDRVSITCKASQDVRNTVAWYQQKTGQSPKLLIYSASYRNTGVPDRFTGSISGTDFTFTISSVQAEDLALYYCQQHYSTPYTFGGGTKLEVKRADAAPTVSIFPPSSEQLTSG

SEQ ID NO: 52 - skipped

SEQ ID NO: 53 - protein, 143 aa, Mus musculus
QVFVYMLLWLSGVDGDIVMTQSQKFMSTSVGDRVSVTCKASQNVGTNVAWYQQKPGQSPKALIYSASYRFSGVPDRFTGSGSGTDFTLTISNVQSEDLAEYFCQQYNSYPYTFGGGTKLEIKRADAAPTVSIFPPSSEQLTSG

SEQ ID NO: 54 - protein, 180 aa, Mus musculus
MAVLVLFLCLVAFPSCVLSQVQLKESGPGLVAPSQSLSITCTVSGFSLLSYGVHWVRQPPGKGLEWLGVIWTGGSTNYNSALMSRLSISKDDSKSQVFLKMNSLQTDDTAMYYCARYYYAMDYWGQGTSVTVSSAKTTPPSVYPLAPGCGDTTGSSVTLGCLVKGYFPESVTVTWNSGSL

SEQ ID NO: 55 - skipped

SEQ ID NO: 56 - protein, 7 aa, Homo sapiens
FQTFEGD

SEQ ID NO: 57 - protein, 6 aa, Homo sapiens
QTFEGD

SEQ ID NO: 58 - protein, 13 aa, Homo sapiens
VPDLGRFQTFEGD

SEQ ID NO: 59 - protein, 439 aa, Oryctolagus cuniculus
QSVEESGGRLVTPGTPLTLTCTASGFTISSYHMTWVRQAPMKGLEWIGTISSSGSTYYASWAKGRFTISKTSSTTVDLKITSPATEDTATYFCARSVPGDSSGEIWGRGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 60 - protein, 217 aa, Oryctolagus cuniculus
AQVLTQTASPVSAAVGGTVTINCQSSQSVYNKNWLAWYQQKPGQPPKRLIYSASTLDSGVSSRFKGSGSGTQFTLTISGVQCDDAATYYCQGEFSCSRGDCSAFGGGTEVVVQGDPVAPTVLIFPPSADLVATGTVTIVCVANKYFPDVTVTWEVDGTTQTTGIENSKTPQNSADCTYNLSSTLTLTSTQYNSHKEYTCKVTQGTTSVVQSFNRGDC